SEQUENCE LISTING (18 sequences)

SEQ ID NO: 1 - DNA, 20 bp, Artificial Sequence
  PCR Primer Gapdh forward
atcaagaaggtggtgaagca

SEQ ID NO: 2 - DNA, 21 bp, Artificial Sequence
  PCR Primer Gapdh reverse
agacaacctggtcctcagtgt

SEQ ID NO: 3 - DNA, 21 bp, Artificial Sequence
  PCR Primer Ifnb1 forward
agctccaagaaaggacgaaca

SEQ ID NO: 4 - DNA, 20 bp, Artificial Sequence
  PCR Primer Ifnb1 reverse
gccctgtaggtgaggttgat

SEQ ID NO: 5 - DNA, 19 bp, Artificial Sequence
  PCR Primer Tnf forward
cctgtagcccacgtcgtag

SEQ ID NO: 6 - DNA, 22 bp, Artificial Sequence
  PCR Primer Tnf reverse
gggagtagacaaggtacaaccc

SEQ ID NO: 7 - DNA, 22 bp, Artificial Sequence
  PCR Primer Il10 forward
gctggacaacatactgctaacc

SEQ ID NO: 8 - DNA, 21 bp, Artificial Sequence
  PCR Primer Il10 reverse
atttccgataaggcttggcaa

SEQ ID NO: 9 - DNA, 21 bp, Artificial Sequence
  PCR Primer Cxcl10 forward
ccaagtgctgccgtcattttc

SEQ ID NO: 10 - DNA, 21 bp, Artificial Sequence
  PCR Primer Cxcl10 reverse
tccctatggccctcattctca

SEQ ID NO: 11 - DNA, 19 bp, Artificial Sequence
  PCR Primer Ccl2 forward
tctgggcctgctgttcaca

SEQ ID NO: 12 - DNA, 24 bp, Artificial Sequence
  PCR Primer Ccl2 reverse
cctactcattgggatcatcttgct

SEQ ID NO: 13 - DNA, 21 bp, Artificial Sequence
  PCR Primer Ccl3 forward
tgtaccatgacactctgcaac

SEQ ID NO: 14 - DNA, 21 bp, Artificial Sequence
  PCR Primer Ccl3 reverse
caacgatgaattggcgtggaa

SEQ ID NO: 15 - DNA, 23 bp, Artificial Sequence
  PCR Primer Ccl5 forward
gcccacgtcaaggagtatttcta

SEQ ID NO: 16 - DNA, 20 bp, Artificial Sequence
  PCR Primer Ccl5 reverse
acacacttggcggttccttc

SEQ ID NO: 17 - DNA, 20 bp, Artificial Sequence
  PCR Primer Gmcsf forward
gaagcatgtagaggccatca

SEQ ID NO: 18 - DNA, 20 bp, Artificial Sequence
  PCR Primer Gmcsf reverse
gaatatcttcaggcgggtct

(19) United States
(12) Patent Application Publication
(10) Pub. No.: US 2022/0362153 A1
(43) Pub. Date: Nov. 17, 2022

(54) COMPOSITIONS AND METHODS FOR PULMONARY SURFACTANT-BIOMIMETIC NANOPARTICLES

(71) Applicant: The General Hospital Corporation, Boston, MA (US)

(72) Inventor: Mei Xiong Wu, Lexington, MA (US)

(21) Appl. No.: 17/767,140

(22) PCT Filed: Oct. 6, 2020
(86) PCT No.: PCT/US20/54377

§ 371 Date: Apr. 7, 2022

Related U.S. Application Data
(60) Provisional application No. 62/911,559, filed on Oct. 7, 2019.
(60) Provisional application No. 62/932,552, filed on Nov. 8, 2019.
(60) Provisional application No. 62/961,073, filed on Jan. 14, 2020.

(51) Int. Cl. A61K 9/127; A61K 39/145; A61K 39/39; A61P 31/16

(52) U.S. Cl. CPC A61K 9/1271; A61K 39/145; A61K 39/39; A61P 31/16; A61K 2039/55555

(57) ABSTRACT

Compositions and methods comprising Pulmonary Surfactant (PS)-biomimetic nanoparticles are disclosed. Specifically, the disclosure is related to a composition comprising a nanoparticle with an average size of 200-400 nm, including a plurality of pulmonary surfactant biomimetic molecules, wherein the nanoparticle is negatively charged; and one or more cargo molecules that are enveloped by the nanoparticle, wherein the cargo molecule has a molecular weight up to 1200 Da.

### FEDERALLY SPONSORED RESEARCH OR DEVELOPMENT

This invention was made with Government support under Grant Nos. AI089779, AI070785, and AI097696 awarded by the National Institutes of Health. The Government has certain rights in the invention.

### TECHNICAL FIELD

Described herein are compositions comprising, and methods of preparing and using, pulmonary surfactant-biomimetic nanoparticles, e.g., PS-GAMP.

### BACKGROUND

Current influenza vaccines protect against viral infections primarily by inducing neutralizing antibodies specific for viral surface hemagglutinin (HA) and neuraminidase (NA). However, these surface proteins undergo constant antigenic drift/shift, greatly limiting the efficacy of these vaccines (1). Studies demonstrating the essential role of lung CD8⁺ resident memory T (T_{RM}) cells in heterosubtypic immunity may provide an explanation to this limitation (2, 3). Induced sufficiently by natural viral infections, these cells not only recognize highly conserved internal proteins that are shared amongst heterosubtypic influenza viruses, but are also capable of clearing viruses at the site of viral entrance when their numbers are low (4-6). Similarly, live vector-engineered and attenuated influenza vaccines can induce lung CD8⁺ Tau cells (7, 8), but a delicate balance must be struck between their safety and immunogenicity. Moreover, these replicating vaccines are often compromised by pre-existing immunity and are consequently suitable in only some populations (9). On the contrary, non-replicating influenza vaccines induce poor T cell immunity in the respiratory tract and require potent mucosal adjuvants to overcome the immunoregulatory mechanisms of the respiratory mucosa.

### SUMMARY

Described herein is a safe and potent mucosal adjuvant that can be used, e.g., to augment influenza vaccines.

Other features and advantages of the invention will be apparent from the following detailed description and figures, and from the claims.

In one aspect, the disclosure is related to a composition comprising a nanoparticle with an average size of 200400 nm, including a plurality of pulmonary surfactant-biomimetic molecules, wherein the nanoparticle is negatively charged; and one or more cargo molecules that are enveloped by the nanoparticle, wherein the cargo molecule has a molecular weight up to 1200 Da.

In some embodiments, the pulmonary surfactant-biomimetic molecules comprise 50%-90% of 1,2-dipalmitoyl-sn-glycero-3-phosphocholine (DPPC) by weight, 5%-15% of a negatively charged lipid by weight, and/or 5%-15% of a neutral lipid by weight.

In some embodiments, the negatively charged lipid is 1,2-dipalmitoyl-sn-glycero-3-phospho-(1′-rac-glycerol) (DPPG) and the neutral lipid is cholesterol.

In some embodiments, the nanoparticle further comprises a plurality of polyethylene glycol (PEG) with an average molecular weight of 500-5000 Da. In some embodiments, the polyethylene glycol is linked to an external surface of the nanoparticle.

In some embodiments, the nanoparticle further comprises 5-15% of 1,2-dipalmitoyl-sn-glycero-3-phosphoethanolamine-N-[methoxy(polyethylene glycol)-2000](DPPE-PEG2000) by weight.

In some embodiments, the cargo molecule is a stimulator of interferon genes (STING) agonist.

In some embodiments, the STING agonist is or comprises cyclic Guanosine monophosphate [GMP]-Adenosine monophosphate [AMP] (cGAMP).

In some embodiments, the cGAMP is present in a concentration of 10-100 μg/ml.

In some embodiments, the cargo molecule is long acting-s2-agonists (LABAs) (e.g., formoterol, salmeterol, or vilanterol); cortisosteroids (ICS) (e.g., budesonide, fluticasone propionate, or fluticasone furoate); leukotriene-pathway modulators (e.g., montelukast, or zileuton); inhibitors targeting kinases (e.g., spleen tyrosine kinase, p38 mitogen-activated protein kinase (MAPK), phosphatidylinositol-4,5-bisphosphate 3-kinase (PI3K), Janus kinase (Jak), or phosphodiesterase-4 (PDE4)); agonists or antagonists of receptors (e.g., chemoattractant receptor-homologous molecule expressed on Th2 cells (CRTH2), chemokine receptor 2 (CCR2)); agonists or antagonists of ion channels (e.g., GABA receptor, transient receptor potential cation channel, subfamily A, member 1 (TRPA1), or voltage-gated sodium channel); inducers of IFN-α; long-acting muscarinic antagonists/anticholinergics (LAMAs); inhibitors against IL-5, IL-13, IL-33, or thymic stromal lymphopoietin; CXCR2 antagonists; molecules blocking proinflammatory cytokines (e.g., TNF-α, TNF-β, or IL-6); molecules blocking IL-17/T_{H}17; macrolides; molecules activating HDAC2; STAT6 inhibitors (e.g., AS1517499); anti-virus small molecule drug (e.g., Oseltamivir (Tamiflu), Relenza, or Zanamivir); Favipiravir (T705); agonists for intracellular Toll-like receptor (TLR) TLR3 (e.g. imiquimod, resiquimod (R848), imidazoquinolines (IMQs), motolimod, CU-CPT4a, IPH-3102, or Rintatolimod); agonists for Nodinitib (NOD1), NOD2, NLPR3 or NPLRC3 (e.g., muramyldipeptide (MDP), FK565, or FK156; TLR7 or TLR8 agonists (e.g., Isatoribine, Loxoribine, gardiquimod, AZD8848, IMO-8400, ANA773, IMO-3100, SM360320, or 852A); TLR8 agonists (e.g., VTX-1463, VTX-2337, IMO-8400, or 2,3-Diamino-furo[2,3-c] pyridine); and/or TLR9 agonists (IMO-8400, IMO-3100, SAR-21609, AZD1419, SD-101, IMO-2055, IMO-2125, QAX-935, AVE0675, DIMS0150, MGN-1703, MGN-1706, ISS1018, or Agatolimod).

In one aspect, the disclosure is related to a method of promoting an immune response to an antigen, the method comprising administering to a subject an effective amount of the composition as described herein; and administering to the subject the antigen.

In some embodiments, the subject is a mammal.

In some embodiments, the antigen is enveloped within the nanoparticle; the nanoparticle and antigen are administered in a single composition; or the nanoparticle and antigen are administered in separate compositions.

In one aspect, the disclosure is related to a method of treating a subject who has influenza, the method comprising administering to the subject a therapeutically effective amount of the composition as described herein; and administering to the subject an antigen,

In some embodiments, the cargo molecule is cGAMP and the antigen is an influenza vaccine.

In some embodiments, the subject is a human and the antigen is a human influenza vaccine.

In one aspect, the disclosure is related to a method of treating a subject who has airway disease, the method comprising administering to the subject a therapeutically effective amount of the composition as described herein.

In some embodiments, the cargo molecule is long acting-β2-agonists (LABAs) (e.g., formoterol, salmeterol, or vilanterol); cortisosteroids (ICS) (e.g., budesonide, fluticasone propionate, or fluticasone furoate); leukotriene-pathway modulators (e.g., montelukast, or zileuton); inhibitors targeting kinases (e.g., spleen tyrosine kinase, p38 mitogen-activated protein kinase (MAPK), phosphatidylinositol-4,5-bisphosphate 3-kinase (PI3K), Janus kinase (Jak), or phosphodiesterase-4 (PDE4)); agonists or antagonists of receptors (e.g., chemoattractant receptor-homologous molecule expressed on Th2 cells (CRTH2), chemokine receptor 2 (CCR2)); agonists or antagonists of ion channels (e.g., GABA receptor, transient receptor potential cation channel, subfamily A, member 1 (TRPA1), or voltage-gated sodium channel); inducers of IFN-α; long-acting muscarinic antagonists/anticholinergics (LAMAs); inhibitors against IL-5, IL-13, IL-33, or thymic stromal lymphopoietin; CXCR2 antagonists; molecules blocking proinflammatory cytokines (e.g., TNF-α, TNF-β, or IL-6); molecules blocking IL-17/TH17; macrolides; molecules activating HDAC2; STAT6 inhibitors (e.g., AS1517499); anti-virus small molecule drug (e.g., Oseltamivir (Tamiflu), Relenza, or Zanamivir); and/or Favipiravir (T705).

In some embodiments, the subject is a human and the airway disease is one or a combination of asthma, chronic obstructive pulmonary disease (COPD), allergy, or lung viral infection.

In one aspect, method of treating a subject who has cancer, the method comprising administering to a subject a therapeutically effective amount of a composition as described herein. In some embodiments, the cargo molecule is a chemotherapy agent.

In some embodiments, the subject is a mammal.

In some embodiments, the cancer is a lung cancer and the chemotherapy agent is Gefitinib, Erlotinib, Crizotinib, Everolimus, Afatinib, Crizotinib Doxorubicin, etoposide, Opdivo, and/or Trexall.

In some embodiments, the cancer is nasopharyngeal cancer and the chemotherapy agent is Cisplatin, Carboplatin, Gemcitabine, Doxorubicin, and/or D5-fluorouracil (5-FU).

In some embodiments, the cancer is trachea cancer and the chemotherapy agent is etoposide, cisplatin, and/or carboplatin.

In some embodiments, the cancer is bronchial cancer and the chemotherapy agent is etoposide, cisplatin, carboplatin, 5-FU, docetaxel, paclitaxel, and/or epirubicin.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control. Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-1J. PS-GAMP uptake by AMs requires SP-A and SP-D. (A) A schematic diagram of PS-liposomes labeled with SRB and DiD. (B-E) Free SRB (20 μg) or SRB-DiD-nano4 or SRB-DiD-nano5 (20 μg SRB) was i.n. administered to mice, followed 12 h later by flow cytometric analysis of SRB⁺ and/or DiD⁺ pulmonary cells. The percentages of SRB⁺ cells that were also CD11c⁺ AMs (red) or CD11c⁻ AECs (blue) were analyzed (B) and quantitated (C and D) (n=4). (E) A representative overlay flow cytometry plot of AM and AEC staining for DiD and SRB. (F) AMs were isolated from MHC II-GFP mice and incubated with DiD-nano4 or DiD-nano5 for 4 h after pre-incubation with (low panel) or without (upper panel) PS for 30 min. Scale bar: 10 μm. Alternatively, AMs were isolated from wildtype (WT) mice and incubated for 4 h with DiD-nano4 that was pretreated with WT or Sftpa1^{-/-}Sfpd^{-/-}PS for 30 min (I). The cells were imaged by fluorescent microscopy and quantified for DiD fluorescence intensity in individual cells with Image J (G and I). n=18-36. (H) Lungs were visualized by fluorescent microscopy 12 h after receiving DiD-nano4 or DiD-nano5. Scale bar: 50 μm. (J) DiD-nano4 was i.n. administered to WT or Sftpa1^{-/-}Sftpd^{-/-} mice. CD11c⁺CD11b⁻CD24⁻ AMs were analyzed 12 h later for DiD⁺. n=6. Each symbol represents individual mice in (C, D, and J) or cells in (G and I). The results were presented as means±SEM. Statistical analysis, one-way ANOVA for (C, D, G, and I), and Student's t-test for (J). **p<0.01 and ***p<0.001 between indicated groups. All experiments were repeated three times with similar results.

FIGS. 2A-2L. Adjuvanticity of PS-GAMP. (A and B) Swiss Webster mice were i.n. immunized with VN04 H5N1 vaccine plus 20 μg of free cGAMP or PS-GAMP containing an indicated amount of cGAMP. Ag-specific serum HAI (A) and BALF IgA (B) titers were measured 2 weeks later. n=8. (C to E) C57BL/6 mice were i.n. immunized with VN04 H5N1 vaccine in presence or absence of PS-GAMP (20 μg cGAMP) on d 0 and boosted on d 14. Sera were collected on d 14 (prime) or 21 (Boost) and measured for Ag-specific IgG (C), IgG2c (D), and IgG1 (E) titers. n=4. (F to L) C57BL/6 mice were i.n. immunized with CA09 H1N1 vaccine with or without 20 μg of PS-GAMP or poly IC. Serum IgG (F), BALF IgA (G), and serum HAI (H) titers were measured 2 weeks later. (I-J) Splenocytes were isolated 7 d post-immunization and stimulated with the CA09 H1N1 vaccine. CD8⁺ (1) and CD4⁺ (J) T cells producing IFN-γ after viral Ag stimulation were determined by flow cytometry. (K and L) Survival curves (K) and body weight changes (L) of un-immunized mice (black) or mice that received a single immunization of vaccine alone (green), the vaccine combined with polyIC (blue) or PS-GAMP (red) were challenged 28 d later with 10×LD₅₀ CA09 H1N1 virus. n=6. The results are presented as means±SEM. Each symbol represents individual mice in (A to J). Statistical analysis, one-way ANOVA for (A to J), two-way ANOVA for (L), and Log-rank test for (K). *p<0.05, **p<0.01, and ***p<0.001 in the presence or absence of PS-GAMP. ns, no significance. All experiments were repeated twice with similar results.

FIGS. 3A-3I. CD8⁺ T cell responses induced by PS-GAMP. (A) Numbers of CD4⁺ and CD8⁺ T cells, NK cells, and CD11b⁺ and CD11b⁻ DCs in the lung (upper) and MLN (lower) were analyzed by flow cytometry at an indicated d after mice were i.n. administered with PS-GAMP. n=4. (B) CD11b⁺ mono-DCs and CD11b⁺ tDC were quantified by flow cytometry in the lung and MLN at an indicated d after mice were i.n. immunized with PS-GAMP or infected with 1×LD₅₀ CA09 H1N1 virus. n=4. (C to E) Mice were i.n. vaccinated with OVA-AF647 with or without PS-GAMP. DCs capturing OVA were enumerated in the MLN 36 h post-immunization (C). n=6. The mean fluorescence intensity (MFI) of CD40 (E) or CD86 (F) on these DCs was quantified by flow cytometry. n=4. (F and G) Mice were i.n. immunized with CA09 H1N1 vaccine with or without PS-GAMP or PBS alone as unimmunized controls. CD8⁺ T cells in the lung and MLN were analyzed at indicated d post-immunization for their Ag-specificity by staining with NP₃₆₆₋₃₇₄ tetramer. n=4-8. (H) Mice were immunized as described in (F and G) and challenged 2 d later with 10×LD₅₀ CA09 H1N1 virus. BALF and lung cells were enumerated for GB⁺CD8⁺ T cells at indicated d post-immunization. n=4. (I) Mice were similarly immunized and challenged as (H), except that 20 μg of poly IC or Pam2CSK4 was used in place of PS-GAMP for immunization. GB⁺CD8⁺ T cells were counted 4 d post-challenge as (H). n=4. Each symbol represents individual mice in (A, C-E, and I). The results were presented as means±SEM. Statistical analysis, one-way ANOVA for (A, B, and I), two-way ANOVA for (F to H), and Student's t-test for (C to E). *p<0.05; **p<0.01, and ***p<0.001 compared to d 0 (A and B), influenza vaccine alone (F to H), or between indicated groups. All experiments were repeated twice with similar results.

FIGS. 4A-4J. PS-GAMP-mediated early protection. (A-B) Survival rates of immunized C57BL/6 mice after 10 LD₅₀ CA09 H1N1 viral challenge. (A) The mice were i.n. immunized with CA09 H1N1 vaccine (0.5 μg HA) and PS-GAMP (20 μg cGAMP) on d 2, 4, 6, 8, or 14 before viral challenging as depicted in FIG. 28A. n=6-11. (B) Mice were immunized and challenged either on the same day (0) or 2 d (−2) post-immunization. n=6. (C) Mice were immunized and challenged 2 d later as (A). CD8 T cells were depleted in some mice by injections of anti-CD8 antibody 2 d before and 0, 2, and 4 d after vaccination. n=4. (D) Survival rates of mice immunized with VN04 H5N1 vaccine plus PS-GAMP at indicated d prior challenge on d 0 with 10×LD₅₀ rgVN04 H5N1 virus as depicted in FIG. 28A. n=4-8. (E) Survival rates of mice immunized with VN04 H5N1 vaccine, PS-GAMP, or the vaccine plus free cGAMP, CT, or PS-GAMP, followed with rgVN04 H5N1 viral challenge 2 d later. n=4-8. (F) Mice were i.n. immunized with H7-Rel H7N9 vaccine and 20 μg of PS-GAMP or poly IC and challenged 2 d later by a clinically isolated SH13 H7N9 virus at 40×LD₅₀. n=8-12. (G to J) Ferrets were i.n. immunized with CA09 H1N1 vaccine (9 μg) with or without 200 μg of PS-GAMP and challenged with 10⁶ TCID₅₀ CA09 H1N1 virus 2 d later. Body weight (G), disease score (H), temperature (I), and viral titers in nasal wash (J) were monitored for 12 d. n=4. The results were presented as means±SEM. *p<0.05, **p<0.01, and ***p<0.001 compared to d 0 (A, D), vaccine alone (B, E, and F), or in the presence or absence of anti-CD8 antibody (C). Mouse experiments were repeated twice with similar results. As for ferrets, * indicates significance between PBS and vaccine+PS-GAMP and # indicates significance between vaccine and vaccine+PS-GAMP. *, #p<0.05; **, ##p<0.01; and ***, ###p<0.001. Statistical analysis, two-way ANOVA for (C, G, I, and J), Kruskal Wallis test for (H), and the Log-rank test for (A, B, and D-F).

FIGS. 5A-5N. AECs make an indispensable contribution to PS-GAMP adjuvanticity. (A) Mice were i.p. administered CBX once a day for 3 consecutive days, after which SRB-nano4 was i.n. given to the mice. SRB⁺ AMs (red) and AECs (blue) were analyzed 12 h later and percentages of these cells were shown in (B and C). n=6. (D) Mice were i.p. administered with CBX, tonabersat, or meclofenamate and i.n. immunized with CA09 H1N1 vaccine with or without 20 μg of poly IC or PS-GAMP. Sera were collected 14 d later and analyzed for IgG2c. n=6. (E) Mice were immunized with CA09 H1N1 vaccine and PS-GAMP in the presence or absence of CBX as (D). Lung CD11b⁺ DCs were counted 24 h later. n=4. (F and G) Mice receiving an indicated gap junction inhibitor were immunized as (D) and challenged with 10×LD₅₀ CA09 H1N1 virus 2 d later. GB⁺CD8⁺ T cells in BALF (F) and the lung (G) were analyzed by flow cytometry. n=4. (H) A schematic diagram of generating chimeric mice. Mice were administered lethal irradiation prior to bone marrow (BM) cell transfer. Chimeras were confirmed after 3 months (FIG. 32), immunized, and challenged as (F). Four d after challenge, GB⁺CD8⁺ T cells were enumerated by flow cytometry in BALF (I) and lung (J) and body weight change relative to d 0 (K) and lung viral titers (L) were measured in these mice. n=4-7. (M and N) A correlation between the number of GB⁺CD8⁺ T cells and viral titers was determined by regression analysis. The results were presented as means f SEM. Each symbol represents individual mice. Statistical analysis, one-way ANOVA for (D, F, G, and I-L), Student's t-test for (B, C, and E). *p<0.05, **p<0.01, and ***p<0.001. All experiments were repeated twice with similar results.

FIGS. 6A-6O. PS-GAMP broadens cross-protection against heterosubtypic influenza A viruses. (A to H) Mice were i.n. immunized with CA09 H1N1 vaccine except for SH09 H1N1 vaccine in (G and H) or the vaccine plus PS-GAMP and challenged 2 d (first panel) or 2 weeks (second panel) later with 5×LD₅₀ distant PR8 H1N1 virus (A and B) and heterosubtypicAichi H3N2 (C and D), rgVN04 H5N1 (E and F), or SH13 H7N9 virus (G and H). n=6-7 for (A to F) and n=8-13 for (G and H). (I) Mice were immunized as (A) and challenged 2 d later by 10×LD₅₀ oseltamivir-resistant NC09 H1N1 virus. Unimmunized mice were treated with oseltamivir (20 mg/kg/day) 6 h before the challenge and then daily after viral challenge until the end of the study. The treated mice were challenged by either 10×LD₅₀ CA09 H1N1 or NC09 H1N1 virus. n=6. (J) Mice were immunized with 2018-19 trivalent seasonal influenza vaccine (SIV18-19) alone or alongside PS-GAMP and challenged 1 month later with 5×LD₅₀ mismatched GZ89 H3N2 virus. n=6-12. (K) Mice were immunized with CA09 H1N1 vaccine alone or together with PS-GAMP and challenged 6 months later with 5×LD₅₀ heterosubtypic rgVN04 H5N1 virus. Alternatively, mice were infected with 1-LD₅₀ PR8 H1N1 virus and the mice that survived the infection were challenged again 6 months later with 5×LD₅₀ rgVN04 H5N1 virus for comparison (pre-infection). n=6-7. (L to 0) Ferrets were i.n. immunized with inactivated Perth H3N2 vaccine (15 μg) with or without PS-GAMP (200 μg). Thirty days after immunization, ferrets were challenged with 10⁶ TCID₅₀ heterosubtypic Michigan15 H1N1 virus. Body weight (L), disease score (M), temperature (N), and viral titers in the nasal wash (0) were monitored for 12 d. The results were presented as means±SEM. Mice: *p<0.05, **p<0.01, and ***p<0.001 compared to unimmunized mice. Experiments with mice were repeated twice with similar results. As for ferrets, * indicates significance between PBS and Vaccine+PS-GAMP and # indicates significance between Vaccine and Vaccine+PS-GAMP. *, # p<0.05; **, ##p<0.01; and ***, ### p<0.001. Statistical analysis, two-way ANOVA for (L, N, and O), Kruskal-Wallis test for (M), and the Log-rank test for (A to K).

FIGS. 7A-7K. PS-GAMP fabrication and characterization. (A) A schematic diagram of PS-GAMP fabrication. The liposomes were synthesized in the basis of PS ingredients of mammals, which typically consists of 90% lipids and 10% proteins and is evolutionally conserved. The lipids contain 8-10% of cholesterol, 60-70% of zwitterionic phosphatidylcholines (PC), mainly dipalmitoylated phosphatidylcholine (DPPC), up to 8-15% of anionic phosphatidylglycerol (DPPG), and a relatively small portion of other lipids (17). PEG2000 was utilized in place of hydrophilic proteins and DPPG was replaced with cationic DPTAP in nano3 and nano5 to determine the importance of charges. These PS lipids and PEG2000 form liposomes with a single lipid bilayer encapsulating cGAMP by reverse-phase evaporation as detailed in Materials and Methods. (B to E) Swiss Webster mice were i.n. immunized with VN04 H5N1 vaccine (1 μg HA content) plus 10 μg free cGAMP or an equal amount of cGAMP packaged in the indicated liposomes. Serum IgG (B) and bronchoalveolar lavage fluid (BALF) IgA (C) were measured two weeks later, body weight was monitored for 7 d after immunization (D) and the area under the curve (AUC) was calculated from (D) by PRISM software (E). n=5. Sizes (F), encapsulation rate (G), and zeta potentials (J) of indicated liposomes were measured. (H and I) Free cGAMP or cGAMP-encapsulated liposomes were cultured with BMDCs (H) and BMMs (I) at a final cGAMP concentration of 10 μg/ml for 8 h, after which IFN-β (Ifnb1) was measured by real-time RT-PCR. n=4. (K) STING-deficient mice (Sting^{-/-}) (Red) or wild-type (WT) (Blue) control mice were i.n. immunized with VN04 H5N1 vaccine alone or together with PS-GAMP and serum IgG titers were measured 2 weeks later as above. n=4. The results were presented as means±SEM. Each symbol represents individual mice in (B, C, E, and K) or independent duplicates in (G, H, and I). Statistical analysis, one-way ANOVA for (B, C, E, and H-K), two-way ANOVA for (D). *p<0.05, **p<0.01, and ***p<0.001 compared to vaccine alone group or between indicated groups. All experiments were repeated twice with similar results.

FIGS. 8A-8F. Kinetics of nanoparticle uptake in different tissues. Mice were i.n. administered PBS, free SRB, SRB-encapsulated and DiD-labeled nano4 (Nano4-SRB) or SRB-encapsulated and DiD-labeled nano5 (Nano5-SRB) and analyzed by flow cytometry at varying times. (A) Representative flow plots for SRB⁺ cells in the brain (upper), nasal tissue (middle), and MLN (lower). Data are representative of two separate experiments each assayed in triplicate. (B and C) SRB⁺ cells in the lungs of mice receiving nano4-SRB (B) or nano5-SRB (C). Alveolar macrophages (AM), interstitial macrophages (IM), CD11b⁺DCs, and CD11b⁻DCs were gated as FIGS. 9A-9D. (D-F) SRB⁺ cells in MLN (D), brain (E), or nasal tissue (F) of mice receiving nano4-SRB or nano5-SRB. n=4. The results were presented as means±SEM. Statistical analysis, two-way ANOVA for (B-F). *p<0.05, **p<0.01, and ***p<0.001. The experiment was repeated twice with similar results.

FIGS. 9A-9D. Gating strategy for flow cytometric analysis of cells isolated from indicated tissues. (A) NK cells were identified by NK1.1⁺ and CD3⁻ in pulmonary cells and CD3⁺ cells were separated into CD4⁺ and CD8⁺ T cells. Pulmonary CD11c⁻ cells were divided into neutrophils as CD11b⁺Ly6C⁺Ly6G⁺, whereas inflammatory monocytes were recognized as CD11b⁺Ly6C^{hi}Ly6G⁻. On the gate of CD11c⁺ cells, four populations were discriminated with CD24 and CD11b markers, among which AMs were CD24⁻CD11b⁻Siglec F⁺, IMs were CD24⁻CD11b⁺, CD24⁺CD11b⁻DCs were CD103⁺ MHC II⁺, and tissue-resident CD24⁺CD11b⁺ DCs also expressed MHC II but not CD103. (B) During influenza virus infection or after PS-GAMP administration, CD11b⁺ DCs could be separated into monocyte-derived DCs (Mono-DCs) or tissue resident-like DCs (tDCs). Mono-DCs were Ly6C^{hi} and MHC II expression varied with their activation status. On the other hand, tDCs were Ly6C^{lo}MHC II^{hi}. (C and D) Gating strategy for CD11b⁺DCs and CD11b⁻DCs in MLN (C) or DCs in nasal tissue (D). Gating strategies for T cells, NK cells, neutrophils, and monocytes in MLN, nasal tissue, and brain were similar to those in the lung (A).

FIGS. 10A-10B. Analysis of cells capturing PS-liposomes in the lung. (A) Mice were i.n. administered nano4-SRB prepared as FIG. 1A. Twelve h later, CD11c⁺SRB⁺ cells were characterized mostly as CD24⁻CD11b⁻ AMs, and CD11c⁻SRB⁺ cells were mostly EpCAM⁺CD11b⁻ AECs, which were also positive for MHC II. (B) AMs, CD103⁺ DCs, and CD11b⁺ DCs gated as FIG. 9A were analyzed for direct nanoparticle uptake (SRB⁺ DiD⁺) in mice receiving nano4-SRB. About half of AMs ingested nano4-SRB shown as SRB⁺DiD⁺, whereas DCs rarely captured the nanoparticles. The results were presented as means±SEM. n=4 mice. The experiment was repeated twice with similar results.

FIGS. 11A-11C. Nano4 delivered cGAMP into AMs. (A) Schematic diagrams of DiD-labeled empty PS-mimetic nanoparticles (DiD-PS) and cGAMP-encapsulated PS-mimetic nanoparticles (DiD-PS-GAMP). (B) DiD-PS or DiD-PS-GAMP (20 μg cGAMP) were i.n. inoculated. Pulmonary cells were analyzed for DiD⁺ CD11c⁺ cells by flow cytometry 12 or 36 h later in mice receiving DiD-PS (Red) or DiD-PS-GAMP (Blue). These cells were also assessed for CD40 expression to verify STING activation in the cells. Representative histogram of CD40 expression is given in the middle and Mean Fluorescence Intensity (MFI) is summarized in the right panel. n=4. (C) DiD⁺ (Blue) or DiD⁻ AMs (Red) expressing CD40 were analyzed similarly as (B). n=4. The results were presented as means±SEM. Each symbol represents individual mice in the right panels of B and C. Statistical analysis, t-test for (B and C). **p<0.01 and ***p<0.001 in the presence or absence of cGAMP. The experiment was repeated twice with similar results.

FIG. 12. Positively charged nano5 was entrapped by PS in ex vivo culture. DiD-labeled Nano4 or nano5 was incubated with PS for 30 min. Nanoparticle aggregates on PS were visualized by confocal microscopy. The areas outlined in the 2^{nd} panel were enlarged on the right. BF, Bright Field. Scale bar, 100 μm in panel 1 and 2 and 10 μm in panel 3 and 4. Data are representative of ten similar results in two separate experiments.

FIG. 13A-13C. Nano4 uptake by AMs isolated from non-human primates (NHP). AMs and PS were isolated from rhesus macaques. (A) DiD-nano4 or DiD-nano5 was incubated for 30 min with rhesus macaque PS. Nano5, but not nano4, aggregated on PS and visualized by confocal microscopy. The areas outlined were enlarged in the corresponding panels on the right. Scale bar, 10 μm. Data are representative of five similar results. (B) Monkey AMs were isolated and cultured with DiD-nano4 (upper) or DiD-nano5 (low) for 3 h with or without PS pre-treatment of the nanoparticles and imaged by fluorescent microscopy. Live cells were stained by Calcein-AM and nuclei were stained by Hoechst. Scale bar, 50 μm. DiD fluorescence intensity in cells was quantified by Image J (C). n=221-275. Each symbol represents individual cells. The results were presented as means±SEM. Statistical analysis, one-way ANOVA for (C). ***p<0.001 between indicated groups. We thank Prof. Wanli Liu, Dr. Junyi Wang, and Ms. Shaoling Qi for their help in the NHP study.

FIG. 14. AM capture nano4 in the lung. Lungs were collected 12 h after mice received DiD-nano4 intranasally and frozen thin sections were stained for an AM-specific marker Siglec F and visualized by fluorescent microscopy. Scale bar, 30 μm. The square in the 2^{nd} panel is enlarged on the right panels. Data are representative of six similar results in two separate experiments.

FIG. 15. TEM of nanoparticle distribution in the lung. Nanogold was encapsulated within nano4 (nano4-gold) and nano5 (nano5-gold) as FIG. 7A. Mice were i.n. administered with the nanoparticles. Lungs were collected 6 h later and prepared for TEM. Note: nano4-gold was entrapped within cellular vesicles inside AM (red arrows) and some nanogolds were observed within a cellular vesicle (open red arrows, lower panel). In contrast, nano5-gold was mostly presented on the surface of alveoli (blue arrows). The areas outlined in upper panels are enlarged in the corresponding lower panels. Scale bar, 2 μm for the upper panel and 500 nm for the lower panel.

FIG. 16. AMs from Sftpa1/Sftpd^{-/-} mice had a similar capability as WT AMs in nano4 uptake. SP-A/D are hydrophilic large proteins and well established as a first line of the innate defense. These two collectins are capable of integrating into PS-wrapped bacteria, viruses, cellular debris, apoptotic cells, and various nanoparticles, to facilitate their endocytosis or phagocytosis by AMs (20). To test whether this might be the mechanism for nano4 uptake by AMs, AMs were isolated from WT or Sftpa1/Sftpd^{-/-} mice and incubated with DiD-nano4 that was pre-treated with WT PS for 30 min. DiD fluorescence in cells was captured by confocal microscopy and quantified by Image J software. AMs from Sftpa1/Sftpd^{-/-} mice were found to ingest nano4 as efficiently as WT AMs in the presence of WT PS (FIG. 16), but not in the presence of SP-A/D-deficient PS (FIG. 1I). n=25-32. Each symbol represents individual cells. Statistical analysis, one-way ANOVA. *p<0.05 and **p<0.01 between indicated groups and ns, not significant. The experiment was repeated twice with similar results.

FIGS. 17A-17C. PS-GAMP did not induce overt inflammation in the lung, nose, and central nervous system (CNS). Swiss Webster mice were i.n. administered with PBS, PS-GAMP, VN04 H5N1 vaccine, or the vaccine plus PS-GAMP or CT. (A) Histological examination of the lung (1^{st} and 2^{nd} panel), nose (3^{rd} and 4^{th} panel), and CNS (5^{th} and 6^{th} panel) in 2 d post-immunization. Alveolar and bronchus of lungs, the nasal associated lymphoid tissue of noses, and the olfactory bulb region of the brain tissue are outlined by a dashed rectangle and enlarged in the corresponding bottom panels. The olfactory bulb region of the brain tissue connects directly with olfactory nerves in the nasal cavity. Data are representative of two separate experiments each assayed in triplicate. Scale bars for the lung and nose, the upper panel 400 μm and the lower panel 100 μm. Scale bars for CNS, the upper panel 800 μm and the lower panel 200 μm. (B) Eosinophil infiltrates, epithelium damage, and necrosis of each mouse were analyzed as previously reported (48). The number indicates the number of mice with (+) or without (−) eosinophil infiltrates, epithelium damage or necrosis. n=6 mice. (C) Expression of indicated cytokines and chemokine in the CNS of mice receiving VN04 H5N1 vaccine in the presence or absence of PS-GAMP or CT was determined by real-time RT-PCR 2 d post-immunization. n=2-8. Each symbol represents individual mice. The results were presented as means±SEM.

FIGS. 18A-18V. Alterations of inflammatory and immune cells after PS-GAMP administration or viral infection. C57BL6 mice were i.n. administered with CA09 H1N1 vaccine plus 20 μg of PS-GAMP (Blue) or infected with 1×LD₅₀ CA09 H1N1 influenza virus (Red). Neutrophils, NK, CD4⁺, and CD8⁺ T cells, monocytes, and CD11b⁺ and CD11b⁻ DCs in the lung (A to G) or MLN (H to N) were analyzed by flow cytometry on indicated d post-infection or post-immunization (d.p.i). Neutrophils, NK, CD4⁺, and CD8⁺ T cells, monocytes and DCs in nasal tissue (O to T) or neutrophils and monocytes in brain (U and V) were similarly analyzed. n=4. The results were presented as means±SEM. Statistical analysis, one-way ANOVA. *p<0.05, **p<0.01, and ***p<0.001 compared to d 0. The experiment was repeated twice with similar results.

FIGS. 19A-19C. PS-GAMP did not induce overt inflammation in the lung in contrast to viral infection. Mice were i.n. immunized with CA09 H1N1 vaccine plus 20 μg of PS-GAMP (A) or infected with 1×LD₅₀ CA09 H1N1 influenza virus (B). Lungs were analyzed by H&E staining on indicated d after immunization or infection. Data are representative of two separate experiments each assayed in triplicate. Scale bar, 400 μm for upper panel and 100 μm for lower panel. (C) The lung inflammation was quantified according to a standard scoring system shown on the right (49). n=6. Data were presented as means±SEM. *p<0.05 and ***p<0.001 compared to d 0 by nonparametric test.

FIG. 20. PS-GAMP induces transient production of immune mediators in the lung. Mice were i.n. given 20 μg of PS-GAMP (Blue) or infected with 1×LD₅₀ CA09 H1N1 influenza virus (Red). mRNA levels of indicated mediators were measured by real-time RT-PCR at various time points and normalized against untreated mice. n=4. The results were presented as means±SEM. Statistical analysis, one-way ANOVA. *p<0.05, ** p<0.01, and ***p<0.001 compared to d 0. The experiment was repeated twice with similar results.

FIGS. 21A-21C. PS-GAMP briefly elevates IFN-β protein in BALF. Mice were i. n. administered 20 μg of PS-GAMP (Blue) or infected with 1×LD₅₀ CA09 H1N1 influenza virus (Red). Protein levels of IFN-β (A), TNF-α (B), and IL-10 (C) in BALF were measured by ELISA at various time points. n=4. The results were presented as means±SEM. Statistical analysis, one-way ANOVA. *p<0.05 and ***p<0.001 compared to d 0 (before treatments). The experiment was repeated twice with similar results.

FIGS. 22A-22G. PS-GAMP did not induce any inflammation systemically. Mice were i.n. immunized with CA09 H1N1 vaccine plus 20 μg of PS-GAMP. Body weight (A) and temperature (B) were monitored for 6 d. Mice receiving PBS served as control. n=5. Serum IFN-β (C), TNF-α (D), IFN-γ (E), IL-6 (F), and IL-10 (G) were also monitored for 6 d by ELISA. n=4. The results were presented as means±SEM. The experiment was repeated twice with similar results.

FIGS. 23A-23D. PS-GAMP increases the number of CD11b⁺DCs ingesting extracellular Ag in the lung and MLN. (A) Mice were i.n. vaccinated with OVA-AF647 with or without 20 μg of PS-GAMP. Pulmonary CD11c⁺ cells capturing OVA were analyzed for CD11b and CD24 expression. The numbers in the plots are mean percentages ±SEM of individual cell subsets. (B) Mice receiving OVA (non-fluorescence)+PS-GAMP served as controls to gate out cell activation-related autofluorescence. OVA uptake was analyzed 36 h later on the gate of DCs prepared from MLNs revealing OVA⁺DCs to be mostly CD11b⁺ DCs. (C) DCs did not directly ingest PS-GAMP in the MLN as shown by few CD11c⁺DiD⁺ cells when mice were i.n. administered with 20 μg of DiD-PS-GAMP and analyzed similarly. (D) DiD⁺ cells were also tracked in MLNs from 0 to 60 h after PS-GAMP administration. n=4. The results were presented as means±SEM. The experiment was repeated twice with similar results.

FIGS. 24A-24E. PS-GAMP did not augment Ag-uptake or processing in vivo. Whether PS-GAMP influenced Ag-uptake or Ag-processing was evaluated using AF647-labeled OVA and DQ-OVA. DQ-OVA is OVA conjugation with a BODIPY fluorescent dye (DQ) and remains self-quenched until OVA is proteolytically processed to generate DQ-green fluorescence, which is commonly used to assess Ag-processing. To this end, mice were i.n. administered with PBS (Gray) or AF647-OVA together with DQ-OVA in the presence (Red) or absence (Blue) of PS-GAMP and euthanized 24 h later for flow cytometric analysis (A). AF647-OVA was analyzed on the gate of DC11b⁺ DCs, which were further quantified for OVA cleavage based on DQ-green fluorescence. Percentages and cell numbers of AF64T CD1 IV DCs were summarized in (B) and (C). AF647 and DQ-Green MFIs in these cells were given in (D) or (E), respectively. Each symbol represents individual mice in B to E. The results were presented as means±SEM. Statistical analysis, one-way ANOVA for (B-E). *p<0.05, **p<0.01, and ***p<0.001 in the presence or absence of PS-GAMP. ns, no significance. The experiment was repeated twice with similar results. Note: there was no difference in MFI of DQ-green fluorescence or OVA in AF647⁺CD11b⁺ DCs irrespective of whether or not PS-GAMP was presented (D and E). However, percentages and numbers of CD11b⁺ DCs positive to OVA were robustly increased in the presence of PS-GAMP (B and C), which was attributed primarily from an increased number of CD11b⁺ DCs secondarily to immune mediators induced by PS-GAMP.

FIGS. 25A-25D. PS-GAMP enhances Ag cross-presentation. (A) Mice were i.n. vaccinated with 60 μg of OVA with or without 20 μg of PS-GAMP. Carboxyfluorescein succinimidyl ester (CFSE)-labeled OT-I cells were transferred into vaccinated mice 1 d later. Lungs and MLN were collected 3 d post-cell transfer. (B) OT-I cells were analyzed for Ag-specific proliferation by step-wise decreases of CFSE fluorescence. Inset in the first two panels (PBS and OVA): a reduced scale of the y-axis to show CFSE decreases. Cells of high divisions (≥6, hi) were gated. Numbers of highly divided cells in lungs (C) and MLNs (D) were summarized. n=4-6. Each symbol represents individual mice in C and D. Statistical analysis, one-way ANOVA for (C and D). The results were presented as means±SEM. **p<0.01 and ***p<0.001 in the presence or absence of PS-GAMP. The experiment was repeated twice with similar results.

FIGS. 26A-26C. CD8⁺ T cell responses in the spleen, lung and MLN. C57BL/6 mice were i.n. immunized with CA09 H1N1 vaccine plus 20 μg of PS-GAMP. Mice received PBS as a control. (A) Splenocytes were isolated 7 d post-immunization and stimulated with the CA09 H1N1 vaccine. Representative cytometric profiles of CD4⁺ and CD8⁺ T cells producing IFN-γ are shown. (B) Representative cytometric profiles of NP₃₆₆₋₃₇₄⁺ CD8 T cells in the lung 4 d after immunization. (C) Percentages of PA₂24-233 (Blue) or PB1703-711 (Red) positive cells were determined on gate of CD3⁺CD8⁺ T cells. Each plot is representative of four similar results in the same group. n=4. Data are presented as means±SEM. The experiment was repeated twice with similar results.

FIGS. 27A-27C. Early viral specific GB⁺CD8⁺ T cells and BALF antibodies. (A) C57BL/6 mice were either left unimmunized or immunized with CA09 H1N1 vaccine alone or the vaccine plus 20 μg of PS-GAMP, followed 2 d later by challenging with 10×LD₅₀ CA09 H1N1 virus. Lungs were collected 4 d post-infection. (B) The percentages of NP₃₆₆₋₃₇₄ tetramer (Blue), PB1₇₀₃₋₇₁₁ (Red), or PA₂₂₄₋₂₃₃ (Green) positive cells were obtained on the gate of GB⁺CD8T cells. CD8⁺ T cells from un-immunized/un-challenged mice were analyzed in parallel as negative controls (Gray). n=3. (C) BALF were analyzed for Ag-specific IgA and IgM titers 6 d post-immunization. n=4. Data are presented as means±SEM. The experiment was repeated twice with similar results.

FIGS. 28A-28I. Supplementary data for FIGS. 4A-4J. (A) A schematic diagram of vaccination and viral challenge schedule. (B to F) The body weight changes (B, C, E, and F) or survival (D) of mice corresponding to those described in FIGS. 4A-4E, respectively. (G and H) Mice were i.n. immunized with H7-Rel H7N9 vaccine alone or alongside 20 μg of PS-GAMP or poly IC and challenged 14 d later by a clinically isolated SH13 H7N9 virus. n=10-13. (I) Body weight change of mice described in FIG. 4F. Statistical analysis, two-way ANOVA for (B, C, E, F, H, I), Log-rank test for (D and G). *p<0.05, **p<0.01, ***p<0.001, and #p<0.05. All experiments were repeated at least twice with similar results.

FIGS. 29A-29B. An inverse correlation of SRB⁺AMs vs. SRB⁺AECs over time in vivo while DiD⁺ AMs remained unaltered in percentages. SRB-DiD-nano4 was i.n. inoculated into mice. (A) Percentage changes of SRB⁺AMs and SRB⁺AECs relative to a total number of lung cells were tracked over time after the inoculation. n=4. (B) DiD⁺ AMs were analyzed by flow cytometry 12 and 18 h later following nanoparticle administration. n=4. The results were presented as means±SEM. Statistical analysis, t-test. *p<0.05 and **p<0.01 compared between 18 and 12 h. All experiments were repeated twice with similar results.

FIGS. 30A-30D. Entry of cGAMP from AMs into AECs. (A) Mice were i.p. injected with a gap junction inhibitor CBX or PBS for 3 consecutive d, after which 20 μg of PS-GAMP was i.n. administrated. AMs and AECs were sorted 12 h later and analyzed for Ifnb1 (B) and Gmcsf (C) expression by real-time RT-PCR. mRNA levels were first normalized to Gapdh and then to corresponding cells isolated from naïve mice. n=4. (D) Unsorted lung cells were also analyzed similarly for comparisons. n=8. The results were presented as means±SEM. Each symbol represents individual mice in B to D. Statistical analysis, t-test. *p<0.05 and ***p<0.001 in the presence or absence of CBX. ns, no significance. All experiments were repeated twice with similar results.

FIGS. 31A-31E. Tissue and cell distribution of poly IC. Mice received 20 μg of rhodamine-labeled poly IC intranasally. (A) Lungs were dissected and digested 12 h later for flow cytometric analysis of poly IC uptake by CD11c⁺ and CD11c⁻ subsets. CD11c⁺poly IC⁺ cells were further confirmed to be CD24⁻CD11b⁻ AMs. Poly IC uptake was next analyzed on the gate of EpCAM⁺ AECs (B) or CD11c⁺CD24⁺ DCs (C). MLNs (D) and nasal epithelium and lymphatic tissue (E) were also prepared for single-cell suspensions to determine poly IC uptake. Data are representative of two separate experiments each assayed in triplicate.

FIGS. 32A-32B. Cell reconstitution efficacy after bone marrow (BM) cell transfer. Mice were pre-conditioned with lethal irradiation prior to infusion with BM cells isolated from mice carrying reciprocal CD45 alleles, CD45.1 and CD45.2, a surface biomarker for all leukocytes. Donor cells were distinguished from recipients by a specific antibody for CD45.1 or CD45.2. The transfer efficacy was analyzed by quantifying CD45.1 or CD45.2 expression on leukocytes in various tissues in the recipients after three months of infusion (A) and summarized in (B). Each symbol represents individual mice in (B). n=5-7. The experiment was repeated twice with similar results.

FIGS. 33A-33K. Supplementary data for cross-protection studies. (A to K) Body weight changes corresponding to mice described in FIGS. 6A-6K, respectively. The results were presented as means±SEM. Statistical analysis, two-way ANOVA. *p<0.05, **p<0.01, ***p<0.001, and # p<0.05. All experiments were repeated twice with similar results.

FIGS. 34A-34B. Vaccination with trivalent seasonal influenza vaccine and PS-GAMP induces cross-protective immunity against mismatched influenza B virus. (A) A schematic of the vaccination/sampling schedule. BALB/c mice were immunized with trivalent seasonal influenza vaccine (2018-19) (SIV) alone or together with 20 μg of PS-GAMP and challenged 1 month later with 4-105 TCID₅₀ mismatched Florida06 B virus. (B) Lungs were isolated 4 d after the immunization and analyzed for viral titers. Each symbol represents individual mice in (B). n=8-9. The results were presented as means±SEM. Statistical analysis, one-way ANOVA. **p<0.01 in the presence or absence of PS-GAMP. ns, no significance. The experiment was repeated twice with similar results.

FIGS. 35A-35E. PS-GAMP/inactivated influenza vaccine induces viral-specific lung CD8⁺ T_{RM} cells. (A) A schematic of the vaccination/sampling timeline of OT-1 mouse model. Mice were transferred with OT-1 cells and i.n. immunized 1 h later with OVA in presence or absence of PS-GAMP. Thirty-five d later, mice were i.v. injected with anti-CD8p antibody to exclude circulating CD8⁺ T cells before sacrificed for flow cytometric analysis. (B) Total CD8⁺ T cells in the lung were gated by CD3⁺ and CD8α⁺ (profile not shown) and lung OT-I cells were recognized as CD45.2⁺ and CD8β⁻ (antibody i.v. injected) (1^{st} two panels). OT-I cells with T_{RM} phenotype were identified as CD103⁺CD69⁺CD49a⁺. OT-I cells in the spleen served as the control (Gray). The number of lung OT-I T_{RM} cells were summarized in (C). n=6. (D and E) Mice were i.n. immunized with CA09 H1N1 vaccine in the presence or absence of PS-GAMP. Lungs were isolated 6 months later for flow cytometry. NP₃₆₆₋₃₇₄⁺CD8 T cells were gated and validated for CD103 and CD69 expression (D) and the number of NP₃₆₆₋₃₇₄⁺ CD8⁺ T_{RM} cells were summarized in (E). n=4. NP₃₆₆₋₃₇₄⁺ CD8⁺ T cells in the spleen served as the control. The results were presented as means±SEM. Statistical analysis, t-test for (C), one-way ANOVA for (E). ***p<0.001 in the presence or absence of PS-GAMP. All experiments were repeated twice with similar results.

FIGS. 36A-36D. FTY720 did not affect the cross-protection elicited by influenza vaccine/PS-GAMP. (A and B) Mice were i.n. immunized with CA09 H1N1 vaccine alone or together with 20 μg of PS-GAMP and challenged 1 month later with 5×LD₅₀ GZ89 H3N2 virus. (C and D) Mice were immunized and challenged as A and B except that the mice additionally received daily injections of FTY720 (1 mg/kg/day) from −2 to 14 days after the challenge. n=6-8. Statistical analysis, two-way ANOVA for (B and D) and Log-rank test for (A and C). *p<0.05, **p<0.01, and ***p<0.001 compared to the vaccine alone. All experiments were repeated twice with similar results.

FIGS. 37A-37E. Safety and efficacy of PS-GAMP in ferrets. Ferrets were i.n. immunized with an inactivated viral vaccine (Perth H3N2 15 μg) with or without 200 μg of PS-GAMP. Body weight (A) and temperature (B) of the animals were monitored for 6 d. (C) Sera were collected 4 weeks after the immunization and tested for PerthH3N2-specific IgG titers (C). HAT titers were also measured against PerthH3N2 (D) or MichiganH1N1 (E) viral strains. n=4. Each symbol represents individual animals in C to E. The results were presented as means±SEM. Statistical analysis, one-way ANOVA for (C and D). **p<0.01 compared in the presence vs. absence of PS-GAMP.

### DETAILED DESCRIPTION

The cGAS-cGAMP-STING pathway is an important immune surveillance pathway that is activated in the presence of cytoplasmic DNA, e.g., due to microbial infection or patho-physiological conditions including cancer and autoimmune disorders. Cyclic GMP-AMP synthase (cGAS) belongs to the nucleotidyltransferase family and is a universal DNA sensor that is activated upon binding to cytosolic dsDNA to produce the signaling molecule cyclic GMP-AMP (or 2′-3′-cGAMP or cyclic guanosine monophosphate-adenosine monophosphate). Acting as a second messenger during microbial infection, 2′-3′-cGAMP binds and activates STING, leading to production of type I interferon (IFN) and other co-stimulatory molecules that trigger the immune response. Besides its role in infectious disease, the cGAS/STING pathway has emerged as a promising new target for autoimmune diseases and cancer immunotherapy. DNA fragments present in the tumor microenvironment are proposed to activate cGAS in dendritic cells (DC), followed by IFN-induced DC maturation and activation of a potent and beneficial immune response against cancer cells. In a separate context, dysregulation of the cGAS/STING pathway has been implicated in self DNA triggered inflammatory and autoimmune disorders, such as systemic lupus erythematosus (SLE) and Aicardi-Goutieres syndrome.

There continues to be a dearth of effective mucosal adjuvants despite decades of investigation. 2′-3′-cGMP-AMP (cGAMP), a natural agonist of the stimulator of interferon genes (STING), is a secondary messenger generated in response to DNA viral infections or tissue damage (10, 11). It stimulates the production of type I interferons (IFN-Is), which help determine the magnitude of T-helper 1 (Th1) immune responses, particularly those of CD8⁺ T cells (12, 13). STING agonists are potent adjuvants capable of eliciting robust anti-tumor immunity following intratumoral administration and augmenting intradermal influenza vaccines (13, 14). Using these small, water-soluble agonists as mucosal adjuvants, however, is a challenge. They must be delivered into the cytosol of antigen (Ag)-presenting cells (APCs) and/or alveolar epithelial cells (AECs) without breaching the integrity of the pulmonary surfactant (PS) layer, a mixture of lipids and proteins secreted by type II AECs. This PS layer forms a strong barrier, which separates exterior air from internal alveolar epithelium in alveoli, and prevents nanoparticles and hydrophilic molecules from accessing AECs (15, 16).

Development of a “universal” influenza vaccine that confers protection against not only intrasubtypic variants, but also other subtypes of influenza viruses is highly desirable. However, whether such universal influenza vaccines are achievable remains unclear. It has been long recognized in both humans and animal models that viral infection can stimulate heterosubtypic immunity primarily mediated by CD8⁺ T cells (2, 3, 6). Here, a single immunization with inactivated H1N1 vaccine adjuvanted with PS-GAMP conferred protection against lethal challenges with H1N1, H3N2, H5N1 or H7N9 viruses as early as 2 days (d) post-immunization. This cross-protection was sustained for at least 6 months, concurrent with durable virus-specific CD8⁺ T_{RM} cells in the lung. This was largely due to the fact that PS-GAMP-adjuvant influenza vaccine simulated viral infection-induced immunity, characterized by AEC activation, rapid CD11b⁺ DC recruitment and differentiation, and robust CD8⁺ T cell responses in the respiratory system. PS-GAMP is a standalone adjuvant, compatible with not only inactivated influenza viral vaccines, but also other vaccines, e.g., vaccines comprising cocktails of multiple B and T cell epitopes or influenza vaccine subunits. The ability of PS-GAMP to potentiate non-replicating influenza vaccines for strong heterosubtypic immunity makes it a promising adjuvant for “universal” influenza vaccines if its efficacy is shown in humans. As such, it would offer a significant advantage over “replicating” vaccines.

Distinct from conventional vaccine adjuvants targeting primarily APCs, PS-GAMP activated both AMs and AECs; without wishing to be bound by theory, AEC activation appeared to be crucial for adjuvanticity, as blockades in gap junctions as well as STING deficiency in AECs diminished the adjuvanticity considerably whereas STING deficiency in myeloid cells did not. The pivotal role played by AECs over AMs in orchestrating innate and adaptive immune responses is in agreement with what has been described during the early phase of influenza viral infection (24). The ability of cGAMP to enter AECs without breaching the PS layer was ascribed to SP-A/D-receptor-mediated endocytosis after incorporation of SP-A and SP-D into PS-biomimetic liposomes, which is not feasible in any non-PS-biomimetic liposomes (39-41). In addition, this adjuvant was able to induce robust protection within just 2 d post-immunization, in sharp contrast to current influenza vaccines, which require at least 10-14 d to be effective. Early cross-protection is extremely important to protect first responders and high-risk individuals, especially when antiviral drug-resistant viruses or highly pathogenic viruses such as H5N1 and H7N9 viruses emerge to become pandemics. Because viral spreading can accelerate exponentially after expanding from an epidemic to pandemic early protection during an epidemic would be the most effective means to confine viral spreading and minimize or prevent epidemics becoming pandemics, saving millions of lives (42).

### Pulmonary Surfactant (PS)-Biomimetic Nanoparticle

Provided herein are compositions comprising PS-biomimetic nanoparticles with an average size of 200-400 nm. The nanoparticle includes a plurality of pulmonary surfactant-biomimetic molecules, wherein the nanoparticle is negatively charged; and one or more cargo molecules that are enveloped by the nanoparticle, wherein the cargo molecule has a molecular weight up to 1200 Da.

Provided herein are methods of promoting an immune response to an antigen. The methods include administering to a subject an effective amount of the composition as described herein; and administering to the subject the antigen.

Provided herein are methods of treating a subject who has influenza. The methods include administering to the subject a therapeutically effective amount of the composition as described herein; and administering to the subject an antigen. In some embodiments, the cargo molecule is cGAMP and the antigen is an influenza vaccine.
1. Provided herein are methods of treating a subject who has an airway disease. The methods include administering to the subject a therapeutically effective amount of the composition as described herein, wherein the cargo molecule is long acting-β2-agonists (LABAs) (e.g., formoterol, salmeterol, or vilanterol); cortisosteroids (ICS) (e.g., budesonide, fluticasone propionate, or fluticasone furoate); leukotriene-pathway modulators (e.g., montelukast, or zileuton); inhibitors targeting kinases (e.g., spleen tyrosine kinase, p38 mitogen-activated protein kinase (MAPK), phosphatidylinositol-4,5-bisphosphate 3-kinase (P3K), Janus kinase (Jak), or phosphodiesterase-4 (PDE4)); agonists or antagonists of receptors (e.g., chemoattractant receptor-homologous molecule expressed on Th2 cells (CRTH2), chemokine receptor 2 (CCR2)); agonists or antagonists of ion channels (e.g., GABA receptor, transient receptor potential cation channel, subfamily A, member 1 (TRPA1), or voltage-gated sodium channel); inducers of IFN-α; long-acting muscarinic antagonists/anticholinergics (LAMAs); inhibitors against IL-5, IL-13, IL-33, or thymic stromal lymphopoietin; CXCR2 antagonists; molecules blocking proinflammatory cytokines (e.g., TNF-α, TNF-β, or IL-6); molecules blocking IL-17/T_{H}17; macrolides; molecules activating HDAC2; STAT6 inhibitors (e.g., AS1517499); anti-virus small molecule drug (e.g., Oseltamivir (Tamiflu), Relenza, or Zanamivir); and/or Favipiravir (T705).

Provided herein are methods of treating a subject who has cancer. The methods include administering to a subject a therapeutically effective amount of a composition as described herein. In some embodiments, the cargo molecule is a chemotherapy agent.

The methods described herein can provide improvement of the delivery efficacy of the cargo molecules as described herein by at least 1-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 50-fold, at least 100-fold, at least 200-fold, at least 500-fold, at least 1000-fold compared to a similar method performed without the use of PS-biomimetic nanoparticles.

### Nanoparticles

In some embodiments, the nanoparticle is a liposome, a vesicle, an emulsion, or a micelle.

In some embodiments, the nanoparticle may contain one or more types of surfactants including detergent, wetting agents, emulsifiers, foaming agents, or dispersants. In some embodiments, the surfactant comprises at least one hydrophobic end and/or at least one hydrophilic end. In some embodiments, the surfactant is positively charged, neutral, or negatively charged.

In some embodiments, the surfactant is a lipid. In some embodiments, the surfactant is a phospholipid. In some embodiments, the nanoparticle may comprise 1, 2, 3, 4, 5, 6, 7, 8, 9, 10, or more layers of surfactant. In some embodiments, the nanoparticle is a water-in-oil-in-water emulsion.

In some embodiments, the percent of surfactant in a nanoparticle can range from 0% to 100% by weight, from 5% to 100% by weight, from 10% to 100% by weight, from 15% to 100% by weight, from 20% to 100% by weight, from 25% to 100% by weight, from 30% to 100% by weight, from 35% to 100% by weight, from 40% to 100% by weight, from 45% to 100% by weight, from 50% to 100% by weight, from 55% to 100% by weight, from 60% to 100% by weight, from 65% to 100% by weight, from 70% to 100% by weight, from 75% to 100% by weight, from 80% to 100% by weight, from 85% to 100% by weight, from 90% to 100% by weight, or from or from 95% to 100% by weight. In some embodiments, the percent of surfactant in a nanoparticle can range from 0% to 95% by weight, from 0% to 90% by weight, from 0% to 85% by weight, from 0% to 80% by weight, from 0% to 75% by weight, from 0% to 70% by weight, from 0% to 65% by weight, from 0% to 60% by weight, from 0% to 55% by weight, from 0% to 50% by weight, from 0% to 45% by weight, from 0% to 40% by weight, from 0% to 35% by weight, from 0% to 30% by weight, from 0% to 25% by weight, from 0% to 20% by weight, from 0% to 15% by weight, from 0% to 10% by weight, or from 0% to 5% by weight. In some embodiments, the percent of surfactant in a nanoparticle can be 0% by weight, approximately 1% by weight, approximately 2% by weight, approximately 3% by weight, approximately 4% by weight, approximately 5% by weight, approximately 10% by weight, approximately 15% by weight, approximately 20% by weight, approximately 25% by weight, approximately 30% by weight, approximately 35% by weight, approximately 40% by weight, approximately 45% by weight, approximately 50% by weight, approximately 55% by weight, approximately 60% by weight, approximately 65% by weight, approximately 70% by weight, approximately 75% by weight, approximately 80% by weight, approximately 85% by weight, approximately 90% by weight, approximately 95% by weight, or approximately 100% by weight.

In some embodiments, the nanoparticle as described herein can have an average size from 200 nm to 210 nm, from 210 nm to 220 nm, from 220 nm to 230 nm, from 230 nm to 240 nm, from 240 nm to 250 nm, from 250 nm to 260 nm, from 260 nm to 270 nm, from 270 to 280 nm, from 280 nm to 290 nm, from 290 nm to 300 nm, from 300 nm to 310 nm, from 310 nm to 320 nm, from 320 nm to 330 nm, from 330 nm to 340 nm, from 340 nm to 350 nm, from 350 nm to 360 nm, from 360 nm to 370 nm, from 370 nm to 380 nm, from 380 nm to 390 nm, or from 390 nm to 400 nm.

### Pulmonary Surfactant (PS)

Pulmonary surfactant is a surface-active lipoprotein complex (phospholipoprotein) formed by type H alveolar cells. The proteins and lipids that make up the surfactant have both hydrophilic and hydrophobic regions. By adsorbing to the air-water interface of alveoli, with hydrophilic head groups in the water and the hydrophobic tails facing towards the air, the main lipid component of surfactant, 1,2-dipalmitoyl-sn-glycero-3-phosphocholine (DPPC), reduces surface tension.

Pulmonary surfactant typically consists of 90% lipids and 10% proteins and is evolutionally conserved. The lipids contain 8-10% of cholesterol, 60-70% of zwitterionic phosphatidylcholines (PC), mainly dipalmitoylated phosphatidylcholine (DPPC), up to 8-15% of anionic phosphatidylglycerol (DPPG), and a relatively small portion of other lipids (17).

### Pulmonary Surfactant (PS)-Biomimetic Nanoparticle

In some embodiments, a PS-biomimetic nanoparticle can be a nanoparticle that comprises a plurality of PS-biomimetic molecules, including but not limited to, 1,2-dipalmitoyl-sn-glycero-3-phosphocholine (DPPC), 1,2-dipalmitoyl-sn-glycero-3-phospho-(1′-rac-glycerol) (DPPG), cholesterol, polyethylene glycol (e.g., PEG2000), 1,2-dipalmitoyl-sn-glycero-3-phosphoethanolamine-N-[methoxy(polyethylene glycol)-2000](DPPE-PEG2000), phosphatidylethanolamine, phosphatidylinositol, phosphatidylserine, sphingomyelin, and/or lysophospholipid.

In some embodiments, the PS-biomimetic molecule is a lipid, a protein, a lipoprotein, a phospholipid, or a phospholipoprotein.

In some embodiments, the PS-biomimetic molecule is a domain, a moiety, a portion or a whole molecule of a pulmonary surfactant. In some embodiments, the PS-biomimetic molecule is a natural product. In some embodiments, the PS-biomimetic molecule is artificially synthesized.

In some embodiments, the PS-biomimetic molecule is positively, neutral, or negatively charged. In some embodiments, the PS-biomimetic molecule has at least one hydrophobic end and/or at least one hydrophilic end.

In some embodiments, the PS-biomimetic molecule comprises one or more fatty acid groups or salts thereof, and/or one or more head group. In some embodiments, a fatty acid group may comprise digestible, long chain (e.g., C8-C50), substituted or unsubstituted hydrocarbons. In some embodiments, a fatty acid group may be a C10-C20 fatty acid or salt thereof. In some embodiments, a fatty acid group may be a C15-C20 fatty acid or salt thereof. In some embodiments, a fatty acid group may be a C15-C25 fatty acid or salt thereof. In some embodiments, a fatty acid group may be unsaturated. In some embodiments, a fatty acid group may be monounsaturated. In some embodiments, a fatty acid group may be polyunsaturated. In some embodiments, a double bond of an unsaturated fatty acid group may be in the cis conformation. In some embodiments, a double bond of an unsaturated fatty acid may be in the trans conformation. In some embodiments, the fatty acid group is a palmitic acid. In some embodiments, the head group is a phosphatidylcholine.

### Cargo Molecules of PS-Biomimetic Nanoparticles

Cargo molecules that can be carried in the nanoparticles described herein can include those that have a therapeutic or prophylactic effect on the cells of the lung, e.g., on alveolar epithelial cells (AECs) and/or alveolar macrophages (AMs). Examples include agents (immunostimulants) that enhance an immune response to a co-administered antigen, e.g., to act as an adjuvant to stimulate an immune response; agents (anti-inflammatories or immunosuppressants) that block signaling pathways associated with inflammation, e.g., to suppress inflammation-associated lung diseases including allergy, asthma, and chronic obstructive pulmonary diseases (COPD), inter alia; and anti-cancer agents such as chemotherapeutics. The cargo molecules can be wholly enveloped by the PS (e.g., contained inside a PS membrane forming the outer surface of the nanoparticle), can be mixed into the PS (e.g., in a solid nanoparticle), or can be on the outside/in the membrane/attached to the membrane.

In some embodiments, the cargo molecule can be transferred via gap junctions present between AMs and AECs, and is limited to those small molecules that are small enough to transit the gap junctions. A detailed description can be found in references 29 and 30. Thus, in some embodiments, the cargo molecule can have a molecular weight ranging from 10 Da to 1200 Da, from 50 Da to 1200 Da, from 100 Da to 1200 Da, from 200 Da to 1200 Da, from 300 Da to 1200 Da, from 400 Da to 1200 Da, from 500 Da to 1200 Da, from 600 Da to 1200 Da, from 700 Da to 1200 Da, from 800 Da to 1200 Da, from 900 Da to 1200 Da, from 1000 Da to 1200 Da, or from 1100 Da to 1200 Da. In some embodiments, the cargo molecule can have a molecular weight ranging from 10 Da to 50 Da, from 10 Da to 100 Da, from 10 Da to 200 Da, from 10 Da to 300 Da, from 10 Da to 400 Da, from 10 Da to 500 Da, from 10 Da to 600 Da, from 10 Da to 700 Da, from 10 Da to 800 Da, from 10 Da to 900 Da, from 10 Da to 1000 Da, from 10 Da to 1100 Da, or from 10 Da to 1200 Da. In some embodiments, the cargo molecule can have a molecular weight of approximately 10 Da, 20 Da, 50 Da, 100 Da, 200 Da, 300 Da, 400 Da, 500 Da, 600 Da, 700 Da, 800 Da, 900 Da, 1000 Da, 1100 Da, or 1200 Da.

In some embodiments, the cargo molecule can be an immunostimulant (for use as adjuvants), e.g., stimulator of interferon genes (STING) agonists (e.g., cGAMP, CDN, MK-1454, ADU-S100, E7766); agonists for intracellular Toll-like receptors including TLR3, TLR7, TLR8, or TLR9 (e.g. imiquimod, resiquimod (R848), imidazoquinolines (IMQs), motolimod, CU-CPT4a, IPH-3102, or Rintatolimod); and/or agonists for Nodinitib (NOD1), NOD2, NLPR3 or NPLRC3 (e.g., muramyldipeptide (MDP), FK565, or FK156).

In some embodiments, the cargo molecule can be an anti-inflammatories for airway diseases (e.g., asthma, chronic obstructive pulmonary disease (COPD), or allergy), e.g., long acting-β2-agonists (LABAs) (e.g., formoterol, salmeterol, or vilanterol); cortisosteroids (ICS) (e.g., budesonide, fluticasone propionate, or fluticasone furoate); leukotriene-pathway modulators (e.g., montelukast, or zileuton); inhibitors targeting kinases (e.g., spleen tyrosine kinase, p38 mitogen-activated protein kinase (MAPK), phosphatidylinositol-4,5-bisphosphate 3-kinase (PI3K), Janus kinase (Jak), or phosphodiesterase-4 (PDE4)); agonists or antagonists of receptors (e.g., chemoattractant receptor-homologous molecule expressed on Th2 cells (CRTH2), chemokine receptor 2 (CCR2)); agonists or antagonists of ion channels (e.g., GABA receptor, transient receptor potential cation channel, subfamily A, member 1 (TRPA1), or voltage-gated sodium channel); inducers of IFN-α; long-acting muscarinic antagonists/anticholinergics (LAMAs); inhibitors against IL-5, IL-13, IL-33, or thymic stromal lymphopoietin; CXCR2 antagonists; molecules blocking proinflammatory cytokines (e.g., TNF-α, TNF-β, or IL-6); molecules blocking IL-17/TH17; macrolides; molecules activating HDAC2; and/or STAT6 inhibitors (e.g., AS1517499). A detailed description can be found in Barnes, “Therapeutic approaches to asthma-chronic obstructive pulmonary disease overlap syndromes.” Journal of Allergy and Clinical Immunology 136.3 (2015): 531-545; Glossop et al. “Small-molecule anti-inflammatory drug compositions for the treatment of asthma: a patent review (2013-2014).” Expert opinion on therapeutic patents 25.7 (2015): 743-754, each of which is incorporated by reference in the entirety.

In some embodiments, the cargo molecule can be an anti-virus small molecule drug for treatment of lung viral infection, e.g., flu A and B viruses, respiratory syncytial virus (RSV), rhinoviruses, parainfluenza virus, or Severe Acute Respiratory Syndrome (SARS) coronavirus. The anti-virus small molecule drugs include Oseltamivir (Tamiflu), Relenza, and Zanamivir for inhibiting neuraminidase of flu virus; and Favipiravir (T705) for treatment of various lung viral infections.

In some embodiments, the cargo molecule is a chemotherapy agent against a cancer, e.g., Gefitinib, Erlotinib, Everolimus, Afatinib, and/or Crizotinib for non-small cell lung cancer; Doxorubicin, etoposide, Opdivo, and/or Trexall for small cell lung cancer; Cisplatin, Carboplatin, Gemcitabine, Doxorubicin, and/or D5-fluorouracil (5-FU) for nasopharyngeal cancer; etoposide, cisplatin, and/or carboplatin for trachea cancer; etoposide, cisplatin, carboplatin, 5-FU, docetaxel, paclitaxel and/or epirubicin for bronchial cancer.

In some embodiments, the cargo molecule is a labeling agent, e.g., the nanoparticles can include one or more detectable moieties, e.g., in addition to a cargo molecule, e.g., a fluorescent dye, e.g., a carbocyanine, indocarbocyanine, oxacarbocyanine, thuicarbocyanine, merocyanine, polymethine, coumarine, rhodamine, Sulforhodamine B (SRB), xanthene, fluorescein, a boron-dipyrromethane (BODIPY) dye, or derivatives thereof, including, but not limited to, BODIPY FL, BODIPY R6G, BODIPY TR, BODIPY TMR, BODIPY 581/591, BODIPY 630/650, and BODIPY 650/665, Cy5, Cy5.5, Cy7, VivoTag-680, VivoTag-S680, VivoTag-S750, AlexaFluor660, AlexaFluor680, AlexaFluor700, AlexaFluor750, AlexaFluor790, Dy677, Dy676, Dy682, Dy752, Dy780, DyLight547, Dylight647, HiLyte Fluor 647, HiLyte Fluor 680, HiLyte Fluor 750, IR800 (Dimethyl(4-[1,5,5-tris(4-dimethylaminophenyl)-2,4-pentadienylidene]-2,5-cyclohexadien-1-ylidene)ammonium perchlorate), IRDye 800CW, IRDye 800RS, IRDye 700DX, ADS780WS, ADS830WS, ADS832WS, 1,1-dioctadecyl-3,3,3′,3′-tetramethylindodicarbocyanine, 4-chlorobenzenesulfonate salt (DiD), 1,1′-dioctadecyl-3,3,3′3′-tetramethylindocarbocyanine (DiI, also known as DiIC18(3)), or any other detectable moieties known in the art. The detectable moiety can be, e.g., inside the nanoparticle or outside (e.g., in or linked to the outer surface membrane).

In some embodiments, the cargo molecule is a small molecule or antibody fragment, e.g., an antigen-binding fragments of antibodies.

### STING Agonist

The stimulator of interferon genes (STING) agonist may be any appropriate agonist. In some embodiments, the STING agonist is a nucleic acid, a protein, a peptide, or a small molecule.

In some embodiments, the STING agonist can be a nucleotidic STING agonist or a non-nucleotidic STING agonist.

The nucleotidic STING agonist includes natural cyclic dinucleotides (CDNs), e.g., cGAMP; or synthetic CDNs, e.g., the ‘dithio’ analog ADU-S100 (sulfur-modified phosphodiester linkages on a c-di[AMP] scaffold), or MK-1454. The non-nucleotidic STING agonist includes vascular disrupting agents, e.g., 5,6-Dimethyl-9-oxo-9H-xanthene-4-acetic acid (DMXAA, also known as vadimezan or ASA404); or amidobenzimidazole STING agonists (see WO2019069270A). Other STING agonists are described in WO2015185565A1 (including fluorinated derivatives) and WO2019079261 A1, which are incorporated herein by reference. A detailed description can be found in Marloye et al. “Current patent and clinical status of stimulator of interferon genes (STING) agonists for cancer immunotherapy.” (2019): 87-90, which is incorporated herein by reference.

cGAMP

As used herein, “cGAMP”, or cyclic GMP-AMP, or 2′-3′-cGMP-AMP, refers to cyclic guanosine monophosphate-adenosine monophosphate.

### Antigens

In some embodiments, the nanoparticles include, or are co-administered with, an antigen. In some embodiments, the antigen is a viral antigen.

In some embodiments, the antigen is a respiratory syncytial virus (RSV) antigen. In some embodiments, the antigen is a RSV F protein antigen. In some embodiments, the antigen is a SARS coronaviral (CoV) antigen. In some embodiments, the antigen is the spike (S) protein of SARS-CoV. In some embodiments, the antigen is rhinoviral antigens. In some embodiments, the antigen is parainfluenza antigen

In some embodiments, the antigen is an Influenza virus antigen. In some embodiments, the antigen is an influenza B virus antigen. In some embodiments, the antigen is influenza viral nucleocapsid protein (NP), RNA polymerases PB1, PB2, PA, Hemagglutinin (HA), or neuraminidase (NA) either individually or in various combinations of the proteins.

### Chemotherapy Agent

As used herein, a “chemotherapy agent” is a cytotoxic drug or cytotoxic mixture of drugs that that are intended to destroy malignant cells and tissues. Non-limiting examples of chemotherapeutic agents include one or more alkylating agents; anthracyclines; cytoskeletal disruptors (taxanes); epothilones; histone deacetylase inhibitors; inhibitors of topoisomerase I; inhibitors of topoisomerase II; kinase inhibitors; nucleotide analogs and precursor analogs; peptide antibiotics; platinum-based agents; retinoids; and/or vinca alkaloids and derivatives; or any combination thereof. In some embodiments, the chemotherapeutic agent is a nucleotide analog or precursor analog, e.g., azacitidine; azathioprine; capecitabine; cytarabine; doxifluridine; fluorouracil; gemcitabine; hydroxyurea; mercaptopurine; methotrexate; or tioguanine. Other examples include cyclophosphamide, mechlorethamine, chlorabucil, melphalan, daunorubicin, doxorubicin, epirubicin, idarubicin, mitoxantrone, valrubicin, paclitaxel, docetaxel, etoposide, teniposide, tafluposide, bleomycin, carboplatin, cisplatin, oxaliplatin, all-trans retinoic acid, vinblastine, vincristine, vindesine, vinorelbine, and bevacizumab (or an antigen-binding fragment thereof). Additional examples of chemotherapeutic agents are known in the art.

In some embodiments, a chemotherapy agent can be used for cancer treatment, e.g., Gefitinib, Erlotinib, Everolimus, Afatinib, and/or Crizotinib for non-small cell lung cancer; Doxorubicin, etoposide, Opdivo, and/or Trexall for small cell lung cancer; Cisplatin, Carboplatin, Gemcitabine, Doxorubicin, and/or D5-fluorouracil (5-FU) for nasopharyngeal cancer; etoposide, cisplatin, carboplatin for trachea cancer; etoposide, cisplatin, carboplatin, 5-FU, docetaxel, paclitaxel and/or epirubicin for bronchial cancer.

### Methods of Making PS-Biomimetic Nanoparticles

The nanoparticles described herein can be made using methods known in the art. For example, in some embodiments, 1,2-dipalmitoyl-sn-glycero-3-phosphocholine (DPPC), 1,2-dipalmitoyl-sn-glycero-3-phospho-(1′-rac-glycerol) (DPPG), and 1,2-dipalmitoyl-sn-glycero-3-phosphoethanolamine-N-[methoxy(polyethylene glycol)-2000](DPPE-PEG2000) and cholesterol can be mixed, e.g., with the mass ratio at about 10:1:1:1, or 5-12:0.5-1.5:0.5-1.5:0.5-1.5 dependent on the cargo molecule.

In some embodiments, one or more surfactants can be mixed at any mass ratio known in the art.

The mixture can be dissolved in chloroform, dichloromethane, trichloroethylene, methylchloroform, or other organic solvent known in the art.

In some embodiments, a mixture of lipids was dissolved in a solvent and mixed with a cGAMP solution. The volume ratio between the solvent and the cGAMP solution can be about 1:5, about 1:4, about 1:3, about 1:2, about 1:1, about 2:1, about 3:1, about 4:1, about 5:1, about 6:1, about 7:1, about 8:1, about 9:1, about 10:1 or greater.

In some embodiments, the concentration of cGAMP in the nanoparticle solution can be about 0.1 μg/ml, about 0.5 μg/ml, about 1 μg/ml, about 5 μg/ml, about 10 μg/ml, about 20 μg/ml, about 30 μg/ml, about 40 μg/ml, about 50 μg/ml, about 60 μg/ml, about 70 μg/ml, about 80 μg/ml, about 90 μg/ml, about 100 μg/ml, about 200 μg/ml, about 300 μg/ml, about 500 μg/ml, about 1 mg/ml, about 5 mg/ml, about 10 mg/ml, about 50 mg/ml, about 100 mg/ml, or great.

In some embodiments, trehalose can be added to the nanoparticle suspension at a final concentration of about 1%, about 2%, about 2.5%, about 3%, about 5%, or about 10%.

### Pharmaceutical Compositions and Methods of Administration

The methods described herein include the use of pharmaceutical compositions comprising the nanoparticles described herein as an active ingredient.

Pharmaceutical compositions typically include a pharmaceutically acceptable carrier. As used herein the language “pharmaceutically acceptable carrier” includes saline, solvents, dispersion media, coatings, antibacterial, antiviral, and antifungal agents, isotonic and absorption delaying agents, and the like, compatible with pharmaceutical administration. Supplementary active compounds can also be incorporated into the compositions, e.g., additional adjuvants.

Pharmaceutical compositions are typically formulated to be compatible with its intended route of administration. Examples of routes of administration include nasal (e.g., inhalation).

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions, powders, or suspensions used for intranasal inhalation or sprays can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

For intranasal administration or administration by inhalation, the nanoparticles can be delivered, e.g., in the form of a solution, powder, aerosol, or suspension from a pump spray container that is squeezed or pumped by the subject, or as an aerosol spray presentation from a pressurized container or a nebulizer, optionally with a suitable propellant. Formulations suitable for intranasal administration can be in the form of a dry powder (either alone, as a mixture, for example, in a dry blend with a carrier such as lactose, or as a mixed component particle, for example, mixed with phospholipids, such as phosphatidylcholine) from a dry powder inhaler or as an aerosol spray from a pressurized container, pump, spray, atomizer (e.g., an atomizer using electrohydrodynamics to produce a fine mist), or nebulizer, with or without the use of a suitable propellant, such as 1,1,1,2-tetrafluoroethane or 1,1,1,2,3,3,3-heptafluoropropane.

The pharmaceutical compositions can also be prepared in the form of suppositories (e.g., with conventional suppository bases such as cocoa butter and other glycerides) or retention enemas for nasal delivery.

The pharmaceutical compositions can be included in a container, pack, or dispenser, e.g., in an inhaler, nebulizer, dropper, optionally with instructions for administration for use in a method described herein.

Methods of using PS-Biomimetic Nanoparticles

In some embodiments, the PS-biomimetic nanoparticles can be used to promote a protective immune response to an antigen, e.g., as part of a vaccine, e.g., to treat or reduce the risk of developing a viral or bacterial infection, e.g., influenza (or flu), e.g., in the lungs. In some embodiments, the PS-biomimetic nanoparticles can be used to treat, asthma, respiratory allergies, or chronic obstructive pulmonary disease (COPD), or reduce one or more symptoms of.

In some embodiments, the PS-biomimetic nanoparticles are administered to mucosal (e.g., nasal or lung tissue). In some embodiments, the PS-biomimetic nanoparticles can be administered intranasally (e.g., by an inhaler, nebulizer).

In some embodiments, the PS-biomimetic nanoparticles can be used to increase immune response (e.g., activating innate immunity in the lung; eliciting CD8⁺ T cell responses; protection against viruses, e.g., intrasubtypic protection against influenza viruses; or heterosubtypic protection against influenza viruses).

In some embodiments, the PS-biomimetic nanoparticles can be used as a chemotherapy adjuvant to treat cancer, e.g., lung cancer. In these methods, the cargo is a chemotherapeutic agent, and the methods include administering a therapeutically effective amount of the nanoparticles, e.g., an amount sufficient to result in a reduction in tumor size, tumor number, tumor growth rate, or metastasis.

### EXAMPLES

The invention is further described in the following examples, which do not limit the scope of the invention described in the claims.

### Materials and Methods

PS-GAMP Synthesis

All lipids were purchased from Avanti Polar Lipids, including 1,2-dipalmitoyl-sn-glycero-3-phosphocholine (DPPC), 1,2-dipalmitoyl-sn-glycero-3-phospho-(1′-rac-glycerol) (DPPG), 1,2-dipalmitoyl-3-trimethylammonium-propane (DPTAP), and 1,2-dipalmitoyl-sn-glycero-3-phosphoethanolamine-N-[methoxy(polyethylene glycol)-2000](DPPE-PEG2000). Cholesterol was obtained from Sigma Aldrich. The mass ratio of nano4 and nano6 was DPPC/DPPG/DPPE-PEG/Chol at 10:1:1:1. The lipids were dissolved in 3 ml of chloroform and mixed with 1 ml cGAMP solution (200 μg cGAMP, 13.7 mM NaCl, 0.27 mM KCl, 0.43 mM Na₂HPO4, and 0.147 mM KH₂PO4). Alternatively, cGAMP was replaced with SRB (Sigma Aldrich) and/or 0.5 μmol DiD dye (Life Technologies) was added to the lipid mixture to label cargo or liposome membrane, respectively. The liposomes were synthesized by reverse-phase evaporation (43). In brief, the mixture of lipids and cGAMP was sonicated to achieve a water-in-oil emulsion under N2 for 30 min at 50° C., followed by gentle removal of the solvent via rotary evaporation at a speed of 220 rpm. An excess amount of buffer was added to the mixture and continuously rotated for another 5 min at 50° C. Resultant liposomes were extruded through 400- and 200-nm membranes (Avanti Polar Lipids) at 50° C. The size and zeta potential of liposomes were measured by Zetasizer (Malvern). Encapsulation efficiency was determined by UV absorption of cGAMP at 260 nm in Nanodrop (Life Technologies) and confirmed by liquid chromatography-mass spectrometry (LC-MS) (Agilent). Free cGAMP was removed by a size-exclusion column G-50 (GE Healthcare). To stabilize the liposomes, trehalose was added to the liposome suspension at a final concentration of 2.5%. The resultant suspension was frozen in dry ice/ethanol bath and then lyophilized at −45° C. under vacuum by Freezone 4.5 (Labconco). The lyophilized liposome (PS-GAMP) was stored at −20° C. until use and used in all in vivo studies unless otherwise specified.

### Animals

C57BL/6J and BALB/c mice were purchased from Jackson Laboratories or Shanghai SLAC Laboratory Animal Co., Ltd. Sting-deficient mice (C57BL/6J-Tmem173gt/J), Sftpa1^{-/-}Sftpd^{-/-} mice (B6.Cg-Sftpa1tm2Haw Sftpdtm2Haw/J), C57BL/6 CD45.1 mice (B6.SJL-Ptprca Pepcb/BoyJ), and Swiss Webster mice were attained from Jackson Laboratories or Charles River Laboratories. MHC II-EGFP mice expressing MHC class II molecule infused into enhanced green fluorescent protein (EGFP) was a kind gift of Dr. H. Ploegh, Mass. Institute of Technology. Influenza-free 4-month-old female ferrets were purchased from Marshall BioResources. Healthy naïve 6-year-old male rhesus macaques were obtained from Beijing Institute of Xieerxin Biology Resource, China. The animals were housed in the pathogen-free animal facilities of Massachusetts General Hospital (MGH) or Fudan University in compliance with institutional, hospital, and NIH guidelines. The studies were reviewed and approved by the MGH or Fudan University Institutional Animal Care and Use Committee.

Influenza Viruses and Vaccines

SH13 H7N9 virus (A/Shanghai/4664T/2013), SH09 H1N1 virus (A/Shanghai/37T/2009), and rgGZ89 H3N2 virus consisting of H3 and N2 of A/Guizhou/54/1989 H3N2 virus and A/Puerto Rico/8/1934 (PR8) viral backbone were obtained from Fudan University. Pandemic CA09 H1N1 virus was requested from the American Type Culture Collection (ATCC, #FR-201). PR8 (NR-348), A/Aichi/2/68 H3N2 (Aichi, NR-3177), rgPerth H3N2 [A/Perth/16/2009 H3N2×PR8 (NR-3499)], and B/Florida/4/2006 (Florida06, NR-9696) viral strains were obtained from BEI Resources, NIAID. Reverse-genetically (rg) modified VN04 (rgVN04) H5N1 virus was a kind gift of Dr. R. Webby, St. Jude Children's Research Hospital, which comprised H5 and N1 genes from A/Vietnam/1203/2004 H5N1 virus and a PR8 viral backbone. A/Michigan/45/2015 H1N1 (Michigan15, FR-1483) and antiviral drug-resistant A/North Carolina/39/2009 H1N1 viruses (NC09, FR-488) were acquired from International Reagent Resources, CDC. The viruses were expanded in 10-day-old embryonated chicken eggs (Charles River Laboratories) at 35° C. for 3 d, harvested, purified by sucrose gradient ultracentrifugation, and frozen at −80° C. To challenge mice, the virus was adapted in mice for three cycles of i.n. instillation-lung homogenate preparation and their infectivity in mice was assayed by a 50% lethal dose (LD₅₀) following a standard protocol.

Monovalent CA09 H1N1 vaccine (NR-20347, Sanofi Pasteur, Inc.) and whole inactivated H5N1 vaccine (NR-12148, Baxter AG) were obtained from BEI Resources, NIAID. H7-Rel H7N9 whole inactivated vaccine was a kind gift from Harbin Veterinary Research Institute, Chinese Academy of Agricultural Sciences. Trivalent seasonal influenza vaccine 2018-2019 (SIV 18-19) was attained from Hualan Biological Bacterin Co., Ltd., China. SH09 H1N1 and Perth H3N2 inactivated vaccines were made by inactivation of the viruses with 0.02% formalin for 24 h at 37° C. and purified as above. Ag concentration was quantified by the BCA protein assay and SDS-PAGE based on HA content.

Mouse Immunizations and Challenges

Mice were sedated with ketamine/xylazine and i.n. inoculated with 30 μl (15 μl per nostril) of an indicated influenza vaccine or a mixture of the vaccine and an adjuvant. VN04 H5N1, SIV 18-19, and CA09 H1N1 SV vaccines were employed at a corresponding dose of 1 μg (HA content), 1 μg, or 0.5 μg per mouse, respectively, whereas H7-Rel and SH09 H1N1 vaccines each were administered at 0.25 μg or 3 μg per dose, respectively. Poly IC (Invivogen), Pam2CSK4 (Invivogen), and cholera toxin (Sigma) each were administered at 20, 20, or 10 μg per mouse, respectively. To block gap junctions, CBX, tonabersat, and meclofenamate were obtained from Sigma Aldrich and i.p. injected into individual mice for 4 consecutive days (from 2 d prior to 1 d post-immunization) at corresponding dosages of 25, 10, or 20 mg/kg/day, respectively (31, 32). To deplete CD8⁺ T cells during vaccination and challenge, mice were administered anti-CD8a (53-6.7, BioLegend) antibody 2 d prior and in 0, 2, and 4 d post-immunization at a dose of 200 μg/day. C57BL/6 mice were used for the challenge studies, except for Aichi H3N2, Florida06 influenza B, and GZ89 viruses which challenged Swiss Webster mice or BALB/c mice instead unless otherwise indicated, because C57BL/6 mice were relatively less susceptible to these viruses. To verify antiviral drug resistance of the NC09 virus, unimmunized mice were treated with oseltamivir (20 mg/kg/day) at 6 h before the challenge and then daily until the end of the study. Immunized and control mice were challenged by i.n. instillation of 10×LD₅₀ mouse-adapted homologous virus at an indicated d after immunization, except for H7N9 virus at 40×LD₅₀. However, heterologous viruses each at 5×LD₅₀ were utilized for challenges except for Florida06 influenza B virus at a dose of 4×10⁵ TCID₅₀ as this virus is not lethal to mice. Body weight and survival were monitored daily for 12 d after the challenge.

Ferret Immunizations and Challenges

Four-month-old female ferrets negative to anti-influenza virus antibody were anesthetized by ketamine/xylazine/atropine and i.n. immunized with a vehicle, an influenza vaccine, or a mixture of the vaccine and PS-GAMP. To assay early protection, each ferret receiving 9 μg of CA09 H1N1 vaccine alone or alongside 200 μg of PS-GAMP was challenged with 10⁶ TCID₅₀ CA09 H1N1 viruses 2 d post-immunization. To evaluate cross-protection, each ferret was i.n. immunized with 15 μg of PerthH3N2 vaccine in the presence or absence of 200 μg of PS-GAMP and challenged with 10⁶ TCID₅₀ heterosubtypic Michigan15 H1N1 viruses 30 d post-immunization. Body temperature was monitored by two microchips implanted in each animal (BioMedic Data Systems) and clinical symptoms were scored according to a published protocol (Table 1) (44). Animals were euthanized humanely 2 weeks after viral challenge by sedation and injection of 0.5 ml of Euthanasia-III into the heart.

TABLE 1
Ferret Clinical Symptom Scores (44)

| Score | Nasal symptoms | Activity level (playfulness) |
|---|---|---|
| 0 | No symptoms | Fully playful |
| 1 | Nasal rattling or sneezing | Responds to play overtures but |
| | | does not initiate play activity |
| 2 | Nasal discharge on external | Alert but not playful |
| | nares | |
| 3 | Mouth breathing | Not playful, not alert |

Tissue Processing and Flow, Cytometry

Lungs, nasal tissues, MLNs, and spleens were dissected from indicated mice and processed into single-cell suspensions for flow cytometric analyses. Specifically, the lung and nasal tissues were minced into 1-mm² pieces, digested with 1 ml of collagenase D (2 mg/ml)/DNase 1 (5 mg/ml), both from Roche, at 37° C. for 60 min, and then passed through 40-μm cell strainers (18). To collect BALF, mice were first perfused thoroughly with ice-cold PBS followed by intratracheal lavage with 0.5% BSA in PBS. Single-cell suspensions of the spleen and MLN were prepared by passing the tissues through 40-μm cell strainers directly. After removal of red blood cells in ACK buffer, the remaining cells were washed, blocked by anti-CD16/CD32 antibody (clone 93, 10 μg/ml, BioLegend) for 20 minutes, and stained with fluorescently conjugated antibodies for 30 minutes on ice or NP₃₆₆₋₃₇₄, PA₂₂₄₋₂₃₃, PB1₇₀₃₋₇₁₁ MHC I tetramers for 1 h on ice. Activated T cells were fixed and permeabilized after surface staining, followed by intracellular staining with anti-granzyme B (GB) antibody at 4° C. overnight. Stained cells were acquired on a FACSAria II (BD) and analyzed using FlowJo software (Tree Star). Cell populations and subsets in the mouse respiratory system were gated and analyzed as described (18). The information of various antibodies was given in Table 2.

TABLE 2
Antibodies and Tetramers for flow cytometry

| | | | | Concen- | |
|---|---|---|---|---|---|
| Antibody | Clone | Label | Vendor | tration | |
| NP₃₆₆₋₃₇₄ | N/A | AF647 | NIH | 7.5 | μg/ml |
| MHC I | | | Tetramer | | |
| tetramers | | | Core | | |
| PA₂₂₄₋₂₃₃ | N/A | APC | NIH | 7.5 | μg/ml |
| MHC I | | | Tetramer | | |
| tetramers | | | Core | | |
| PB1₇₀₃₋₇₁₁ | N/A | APC | NIH | 7.5 | μg/ml |
| MHC I | | | Tetramer | | |
| tetramers | | | Core | | |
| anti-Ly6G | 1A8 | APC | Biolegend | 2.0 | μg/ml |
| anti-CD11c | N418 | AF488 | Biolegend | 2.5 | μg/ml |
| anti-CD11b | M1/70 | BV421 | Biolegend | 2.0 | μg/ml |
| | M1/70 | PerCP/Cy5.5 | Biolegend | 2.0 | μg/ml |
| | M1/70 | PE | Biolegend | 2.0 | μg/ml |
| anti-Ly6C | HK1.4 | Pacific Blue | Biolegend | 5.0 | μg/ml |
| | HK1.4 | APC | Biolegend | 2.0 | μg/ml |
| anti-CD24 | M1/69 | Pacific Blue | Biolegend | 5.0 | μg/ml |
| anti-EpCAM | G8.8 | PerCP/Cy5.5 | Biolegend | 2.0 | μg/ml |
| | G8.8 | PE | Biolegend | 1.0 | μg/ml |
| | G8.8 | APC | Biolegend | 2.0 | μg/ml |
| anti-CD40 | 3/23 | PE | Biolegend | 2.0 | μg/ml |
| anti-CD86 | GL-1 | PerCP/Cy5.5 | Biolegend | 1.0 | μg/ml |
| anti-CD8α | 53-6.7 | PerCP/Cy5.5 | Biolegend | 1.0 | μg/ml |
| | 53-6.7 | APC | Biolegend | 1.0 | μg/ml |
| | 53-6.7 | PE | Biolegend | 2.5 | μg/ml |
| | 53-6.7 | AF488 | Biolegend | 5.0 | μg/ml |
| | 53-6.7 | APC/Cy7 | Biolegend | 4.0 | μg/ml |
| anti-CD8β | 53-5.8 | PE/Cy7 | Biolegend | 2.0 | μg/ml |
| | H35-7.2 | PE/Cy7 | eBioscience | 4.0 | μg/ml |
| anti-CD3 | 17A2 | PE | Biolegend | 2.0 | μg/ml |
| | 500A2 | AF647 | eBioscience | 1.0 | μg/ml |
| | 145-2C11 | APC/Cy7 | Biolegend | 2.0 | μg/ml |
| | 17A2 | APC/Cy7 | Biolegend | 4.0 | μg/ml |
| | 500A2 | FITC | Biolegend | 5.0 | μg/ml |
| anti-Granzyme B | GB11 | AF647 | Biolegend | 1 | test |
| | GB11 | Pacific Blue | Biolegend | 1 | test |
| anti-CD4 | GK1.5 | APC/Cy7 | Biolegend | 5.0 | μg/ml |
| | GK1.5 | FITC | Biolegend | 5.0 | μg/ml |
| | GK1.5 | Pacific Blue | Biolegend | 5.0 | μg/ml |
| anti-IFNγ | XMG1.2 | BV421 | Biolegend | 1.0 | μg/ml |
| anti-MHC II | M5/114.15.2 | APC/Fire750 | Biolegend | 4.0 | μg/ml |
| anti-Siglec F | S17007L | APC | Biolegend | 2.0 | μg/ml |
| anti-CD103 | 2E7 | PE/Cy7 | Biolegend | 5.0 | μg/ml |
| | 2E7 | BV421 | Biolegend | 4.0 | μg/ml |
| anti-CD69 | H1.2F3 | BV510 | Biolegend | 2.0 | μg/ml |
| anti-CD49a | HMα1 | PE | Biolegend | 4.0 | μg/ml |
| anti-NK1.1 | PK136 | PE | Biolegend | 2.0 | μg/ml |
| anti-CD45.1 | A20 | PE | Biolegend | 2.0 | μg/ml |
| anti-CD45.2 | 104 | AF647 | Biolegend | 2.5 | μg/ml |

Cytokine and Chemokine Measurements

C57BL/6 mice were i.n. administered 20 μg of PS-GAMP or infected with 1×LD₅₀ CA09 H1N1 virus. Lungs were harvested at indicated times and prepared for total RNA extraction with an RNA purification kit (Roche). To measure cytokines in brains, mice were i.n. administered VN04 H5N1 vaccine (1 μg HA) alone or together with PS-GAMP (20 μg) or CT (10 μg) and sacrificed 48 h later to collect the brain tissue for RNA extraction as above. The RNA was reverse-transcribed (Life technologies) and amplified by real-time PCR using an SYBR Green PCR kit (Roche). Glyceraldehyde 3-phosphate dehydrogenase (GAPDH) served as an internal control. All primers used are listed in Table 3. Murine GM-CSF (eBioscience), IFN-s (Invivogen), TNF-α (BioLegend), IFN-γ (eBioscience), IL-6 (eBioscience), and IL-10 (BioLegend) levels in BALF and serum were measured by specific ELISA kits.

TABLE 3
Primers for Real-time PCR

| Gene | Forward 5′-3′ | |
|---|---|---|
| Gapdh | ATCAAGAAGGTGGTGAAGCA | AGACAACCTGGTCCTCAGTGT |
| Ifnb1 | AGCTCCAAGAAAGGACGAACA | GCCCTGTAGGTGAGGTTGAT |
| Tnf | CCTGTAGCCCACGTCGTAG | GGGAGTAGACAAGGTACAACC |
| | | C |
| Il10 | GCTGGACAACATACTGCTAACC | ATTTCCGATAAGGCTTGGCAA |
| Cxc110 | CCAAGTGCTGCCGTCATTTTC | TCCCTATGGCCCTCATTCTCA |
| Cc12 | TCTGGGCCTGCTGTTCACA | CCTACTCATTGGGATCATCTT |
| | | GCT |
| Cc13 | TGTACCATGACACTCTGCAAC | CAACGATGAATTGGCGTGGAA |
| Cc15 | GCCCACGTCAAGGAGTATTTCTA | ACACACTTGGCGGTTCCTTC |
| Gmcsf | GAAGCATGTAGAGGCCATCA | GAATATCTTCAGGCGGGTCT |

Histology

Swiss Webster mice were i.n. administered PBS, PS-GAMP (20 μg), H5N1 vaccine (1 μg HA), or the vaccine plus PS-GAMP or CT (5 μg). Some mice were infected by CA09 H1N1 virus (250 PFU) as positive controls. Lungs, nasal tissue, and brains were dissected at indicated days after immunization or infection, fixed, and stained using a standard H&E procedure. The slides were scanned and analyzed using a NanoZoomer (Hamamatsu).

Confocal Microscopy

To track DiD-labeled liposomes in the lung, C57BL/6 mice were i.n. administered an equal amount of DiD-nano4 or DiD-nano5. Lungs were excised after 12 h, embedded in an optimal cutting temperature (OCT) compound (Sakura Finetek), and cut into 5-μm frozen sections. The slides were mounted with a ProLong Antifade Mountant containing DAPI (Life Technologies) and imaged by confocal microscopy (Olympus FV1000, UPLSAPO 60XW). To visualize AM uptake of nanoparticles ex vivo, mouse lungs were lavaged six times with 1 ml of PBS containing 0.5% BSA and 5 mM EDTA. The lung lavage was pooled and centrifuged at 220×g. The cells were collected, washed thoroughly by PBS, and cultured in RPMI 1640 medium for 45 min, followed by removal of nonadherent cells. The adherent cells were collected as AMs, suspended at 2×10⁵ cells/ml in medium, and added to 96-well-plates at 200 μl/well. To purify PS, lung lavage was prepared by washing the lung for six times with 1 ml of PBS and centrifuged at 220×g for 10 min to remove cell debris and then at 100,000×g for 1 h to pellet PS. The supernatant (6 ml) was concentrated to 200 μl by 3-kDa Amicon Ultra Centrifugal Filter Units (Merk Millipore) and mixed with PS pellet prepared above. The resultant PS (100 μg total protein) was then mixed with DiD-nano4 or DiD-nano5 (12 μg lipid content in nanoparticles) for 30 min before added to AM cell culture with 4×10⁴ cells in 200 μl of medium. After 4-h incubation under 5% CO₂ at 37° C., cells were stained with a vital dye Calcein-AM (Life Technologies). Uptake of liposomes was quantified by confocal microscopy (Olympus FV1000, UPLSAPO 60XW) followed by ImageJ software analysis.

Statistical Analysis

A two-tailed Student's t-test was used to analyze differences between two groups. ANOVA or Kruskal-Wallis test was used to analyze differences among multiple groups by PRISM software (GraphPad). A p of <0.05 was considered statistically significant. Sample sizes were determined on the basis of preliminary experiments to give a statistical power of 0.8. Most of the experiments were repeated at least twice with similar results. The investigators were not blinded to the experiments which were carried out under highly standardized and predefined conditions, except for microscopy images and H&E slide examinations, which were evaluated in an investigator-blind manner.

Hemagglutination Inhibition (HAI) Assays

Serum samples were collected at indicated times from immunized and control animals and treated with receptor-destroying enzyme (RDE) (Denka Seiken, Tokyo, Japan) at 37° C. for 20 hrs followed by heat inactivation at 56° C. for 30 min. The resultant serum samples were serially diluted and incubated with 4 hemagglutination units (HAU) of an indicated influenza virus at 37° C. for 1 h. The serum-treated virus was incubated with 0.5% chicken red blood cells (for H1N1 and H7N9) or horse red blood cells (for H5N1) at room temperature for 30 minutes. The HAI titer was defined as the reciprocal of the highest serum dilution that inhibited 4 HAU of a given virus.

Enzyme-Linked Immunosorbent Assay (ELISA)

Influenza-specific IgG, IgG1, IgG2a, IgA, and IgG2c antibody titers were measured by ELISA. In brief, 1 μg/ml of recombinant HA was coated onto ELISA plates in NaHCO₃buffer, pH 9.6 overnight, to which serially diluted serum samples were added. Antibody subtypes were quantified by HRP-conjugated goat anti-mouse IgG (NA931V, GE healthcare, dilution 1:6000), IgG1 (1073-05, Southern Biotech, 1:4000), IgG2c (1079-05, Southern Biotech, 1:4000), IgA (A90-103P, Bethyl, 1:10000), IgM (ab97230, 1:20000) or IgG2a (1083-05, Southern Biotech, 1:4000) antibody. Titers of specific antibody subtypes were quantified by using SIGMAFASTM OPD as the substrate and reading the reaction at A490 on a plate reader (Molecular Devices).

Cellular Immune Responses

Splenocytes were isolated from mice 7 d post-immunization by passing the spleens through 40-μm strainers, followed by lysis of red blood cells with ACK (Ammonium-Chloride-Potassium) buffer for 4 min on ice. Cells at 1×106/ml were incubated with influenza vaccine (1 μg/ml) and 4 μg/ml of anti-CD28 (clone 37.51, BD Pharmingen) antibody overnight. Golgi-Plug (BD Pharmingen) was added to the culture and incubated for another 5 h. The stimulated cells were first stained with fluorescence-conjugated antibodies against CD3, CD4, and CD8, followed by intracellular staining with anti-IFN-γ antibody. All antibodies were listed in Table S2. The stained cells were acquired on a FACSAria II (BD) and analyzed using FlowJo software (Tree Star).

Chimeric Mice Generated by Bone Marrow Transplantation

Chimeric mice were generated by bone marrow (BM) transplantation as described (33). Briefly, BM cells were harvested from femur and tibia of gender- and age-matched donor mice different in CD45 alleles. Recipient mice received lethal irradiation from 137Cs gamma irradiator (Mark I, 30 μL. Shepherd) at a dose of 1100 rad administered in two fractions at 3 h apart. Right after the second irradiation, 5×106 donor BM cells were intravenously injected into recipient mice. BM cells of STING-deficient mice (Sting−/− or ST) were transferred to age and gender-matched WT mice or vice versa. WT mice receiving WT BM cells or ST mice receiving ST BM cells were also prepared in parallel. Mice were supplied with antibiotics-containing water from 5 d before irradiation to 14 d after irradiation and housed for 3 months to establish complete reconstitution of donor populations, which was corroborated by flow cytometric analysis of lungs, MLNs, spleens, and peripheral blood mononuclear cells (PBMCs) after staining with anti-CD45.1 (clone A20, BioLegend, 2 μg/ml) or anti-CD45.2 (clone 104, BioLegend, 2.5 μg/ml) antibody.

BM-Derived Dendritic Cells (BMDCs) and BM-Derived Macrophages (BMMs)

BMDCs and BMMs were prepared as previously described (45). Briefly, BM cells were harvested from tibiae and femurs of 4-6-week-old C57BL/6 mice. Cells at a concentration of 1×106/ml were cultured with 10 ng/ml granulocyte macrophage colony stimulating factor (GM-CSF) or macrophage colony stimulating factor (M-CSF) for 7 days to generate BMDCs or BMMs, respectively. CD11c⁺ BMDCs were further purified by high-speed cell sorting in FACSAria 11 (BD).

Requirement of PS for AM Uptake of Nano4 in Non-Human Primates (NHP)

Lungs were surgically removed after rhesus macaques were euthanized, filled with 150 ml of cold RPMI 1640 medium supplemented with antibiotics, immersed in the cold medium, and transported to the laboratory on ice. The AMs and PS were isolated as described (46). Briefly, the filled RPMI 1640 medium was collected from the lung and centrifuged at 200×g to remove cell debris and then at 8000×g for 20 min to pellet PS. The supernatant (30 ml) was concentrated to 1 ml by 10 kDa Amicon Ultra Centrifugal Filter Units (Merk Millipore) and mixed with PS pellet prepared above to obtain concentrated PS with both lipids and surfactant proteins. AMs were isolated by washing the lung six times with 100 ml of PBS containing 0.5 mM EDTA. The lung lavages were pooled and centrifuged at 200×g to collect the cells. The cells were washed thoroughly with PBS and cultured in RPMI 1640 for 20 min, followed by removal of nonadherent cells. The concentrated PS at 2 mg of total proteins was mixed with DiD-nano4 or DiD-nano5 (48 μg lipid content) for 30 min and then incubated with 1.6×105 AMs in 1 ml of medium for 3 h at 37° C. with 5% CO2. AMs were stained with a vital dye Calcein-AM (Life Technologies) and Hoechst (Sigma). AM uptake of the nanoparticles was evaluated by confocal microscopy (Olympus FV3000, UPLSAPO 40×) and analyzed by ImageJ software.

Transmission Electron Microscopy (TEM)

To determine ultrastructural localization of nano4 and nano5 in alveoli, nanogold (5 nm, Alfa Aesar) was encapsulated into nano4 or nano5 by reverse-phase evaporation as described (47). Mice were i.n. administered with nanogold-nano4 or nano5 at an equal amount, 12 h after which lungs were isolated, fixed in Karnovsky fixative at 4° C. overnight, post-fixed in 1% OsO4 in 0.1 M sodium cacodylate buffer for 1.5 h, dehydrated in gradient alcohol series, infiltrated with s-propylene oxide/Epon t812 gradient mixture, and embedded in Epon t812 (Tousimis). Ultrathin sections were cut at 80 nm on a microtome (Reichert-Jung Ultracut E), collected on 100-mesh copper grids, stained with 2% Uranyl Acetate and Lead Citrate (2.66% lead nitrate, 3.52% sodium citrate), and examined on a CM-10 transmission electron microscope (Philips). Digital TEM images were taken by AMT-XR41M 4.0 Megapixel Cooled sCMOS camera (Advanced Microscopy Techniques).

### Example 1: PS-GAMP is Fabricated with PS Constituents

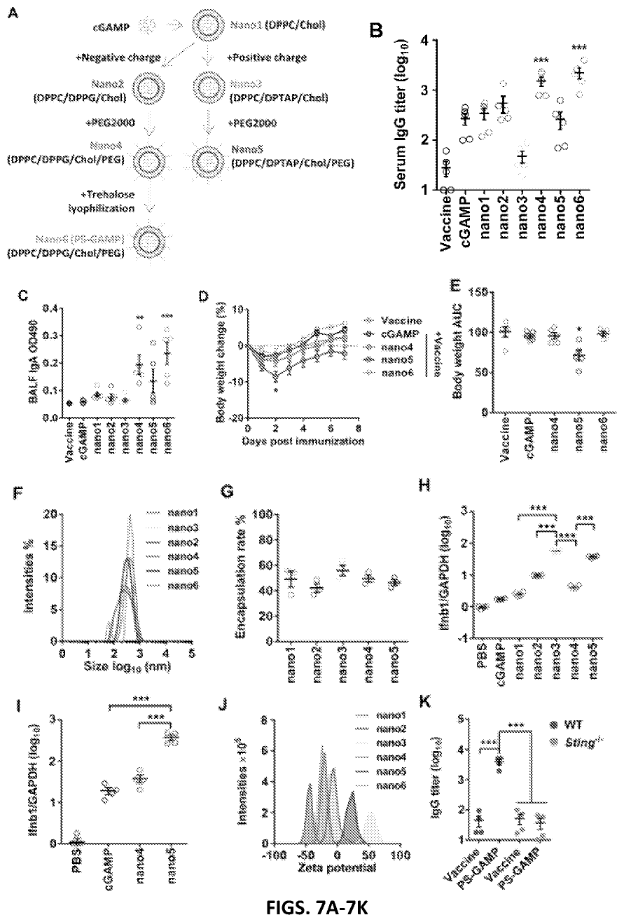

We synthesized a series of liposomes, based on PS constituents (17), to encapsulate cGAMP (FIG. 7A). The negatively charged nano4 was closest to PS in terms of lipid composition and charge. It was the only liposome that, when intranasally (i.n.) introduced alongside whole inactivated A/Vietnam/1203/2004(VN04) H5N1 vaccine, vigorously stimulated the production of serum IgG and bronchoalveolar lavage fluid (BALF) IgA, concomitant with no body weight loss over vaccine alone controls (FIGS. 7B-7E). In contrast, liposomes that were neutral (e.g. nano1), replaced anionic phosphatidylglycerol (DPPG) with cationic 1,2-dipalmitoyl-3-trimethylammonium-propane (DPTAP) (e.g. nano3 or naon5), or lacked PEG2000 (e.g. nano2 and nano3) showed substantially less adjuvanticity while causing significant body weight loss (FIGS. 7A-7E). This was despite their similar size and encapsulation rate to nano4 (FIGS. 7F-7G). Thus, negative charge and PEG2000 appear to play an important role in the function and safety of the liposomes. Unexpectedly, bone marrow-derived dendritic cells (BMDCs) stimulated in vitro with cGAMP encapsulated in positively charged liposomes (nano3 or nano5) expressed higher levels of Ifnb1 than when stimulated with negatively charged liposomes (nano2 and nano4) (FIG. 7H). A similar pattern emerged when bone marrow-derived macrophages (BMMs) were stimulated with positively or negatively charged liposomes encapsulated with cGAMP (FIG. 7I). This highlights the need for in vivo assessments of nanoparticles for their safety and efficacy. Trehalose was then added to the liposome suspension before lyophilization to increase nano4 stability (FIG. 7A). The resultant nano6 liposome, which we termed PS-GAMP, was stable at −20° C. for at least 6 months and exhibited similar zeta potential, size, function, and safety as freshly prepared nano4 (FIGS. 7A, 7F, 7J, and 7B-7E). Moreover, high Ag-specific IgG titers induced by PS-GAMP-adjuvanted influenza vaccine in wild type (WT) but not in STING-deficient mice corroborated that cGAMP, rather than any other constituents, was responsible for PS-GAMP's adjuvanticity (FIG. 7K).

### Example 2: PS-GAMP Uptake by Alveolar Macrophages Requires Surfactant Proteins A and D

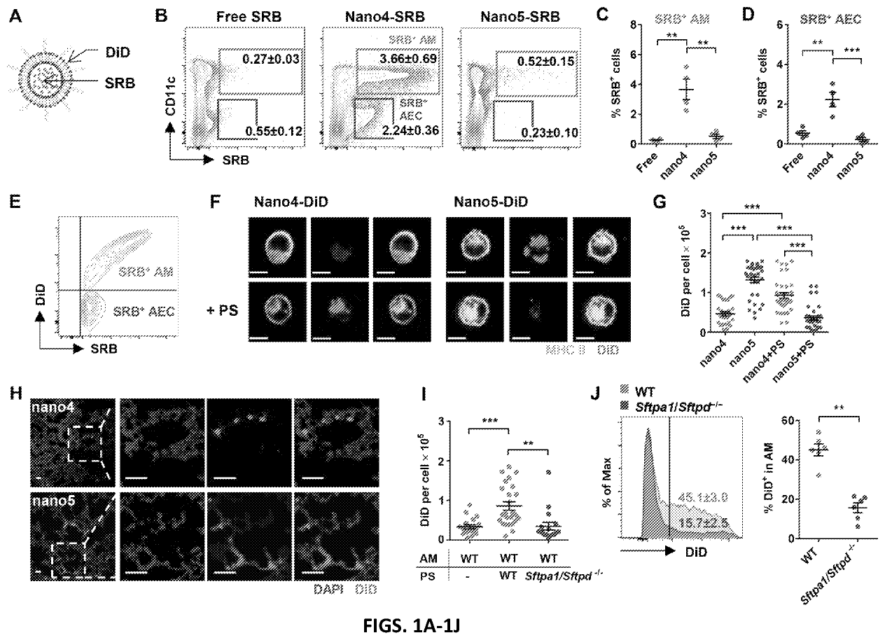

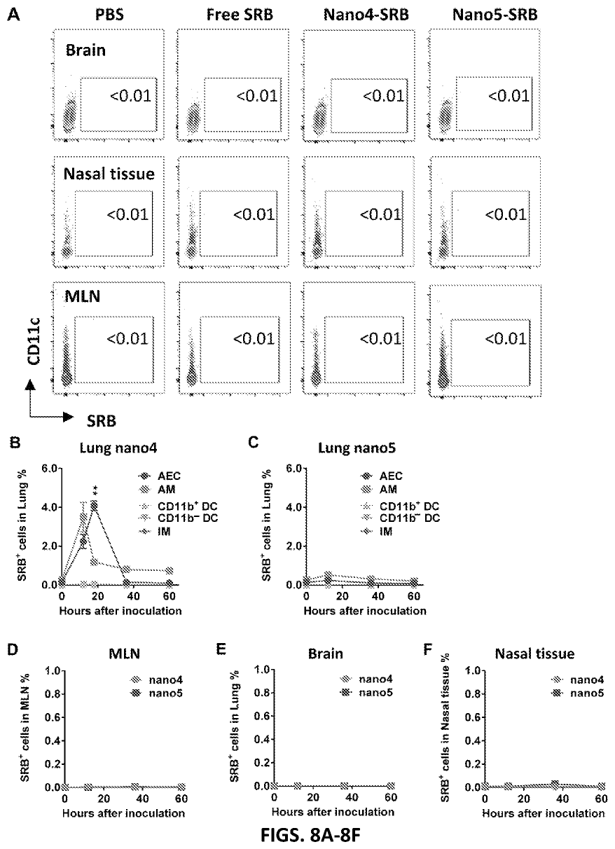

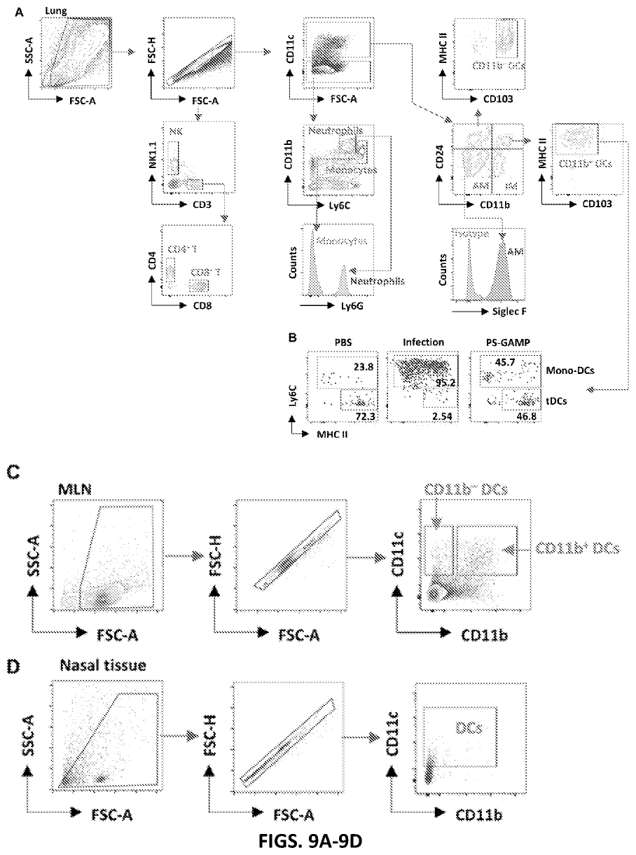

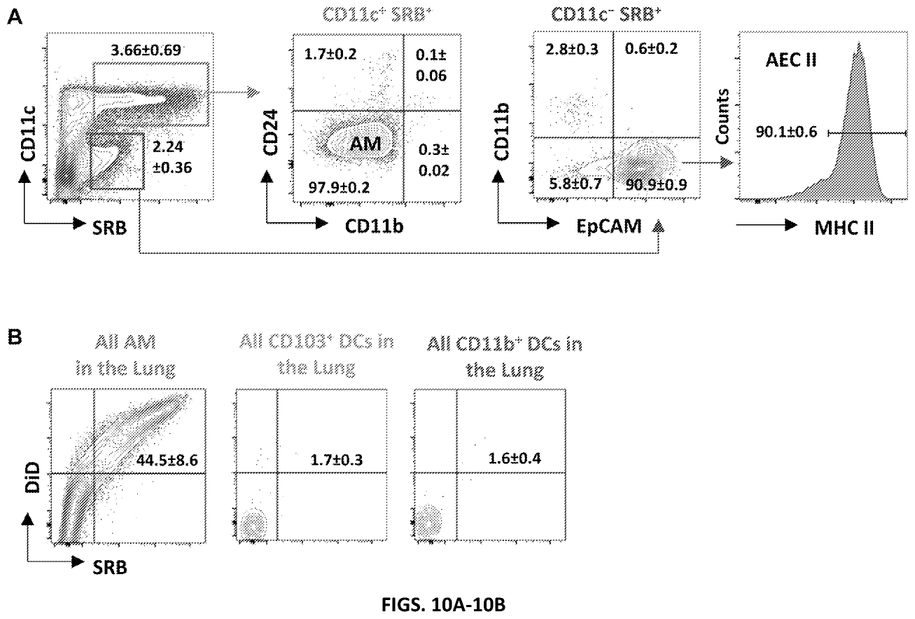

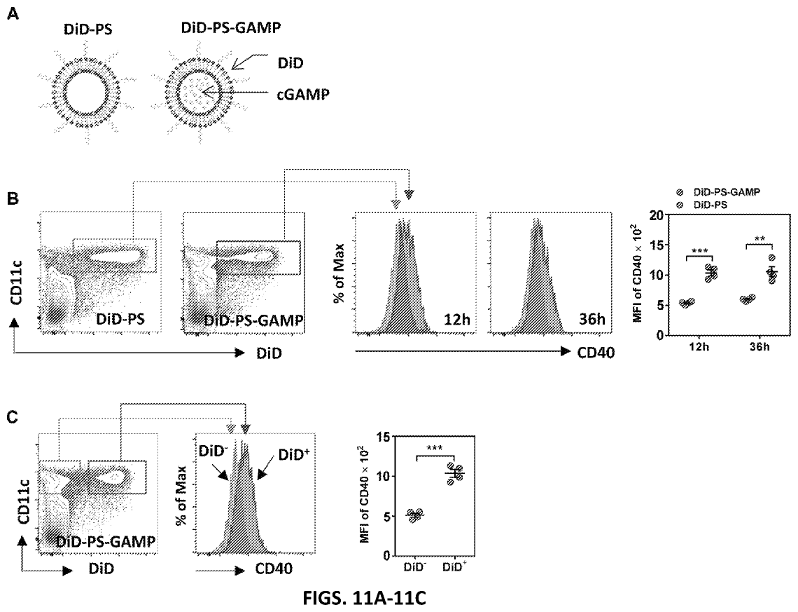

Cellular targets of nano4 and its cargo were next studied by labeling nano4 and nano5 membranes with DiD, a fluorescent lipophilic carbocyanine, and packaging another fluorescent dye with a molecular mass and net negative charge comparable to cGAMP (sulforhodamine B, SRB) within the liposomes (FIG. 1A). The liposomes were i.n. administered to mice and their nasal tissue, brains, mediastinal lymph nodes (MLNs), and lungs were analyzed by flow cytometry at various time points. The lung was the only tissue in which we found SRB⁺ signals over controls (FIG. 1B and FIG. 8A). There, nano4 was taken up directly by CD11b⁻CD11c⁺CD24⁻ alveolar macrophages (AMs) and indirectly by CD11b⁻CD11c⁻EpCAM⁺MHC II⁺ AECs (FIGS. 1B-1D and 10A) (18). Thus, AECs were SRB⁺ but DiD⁻, whereas most of SRB⁺AMs were also DiD⁺, suggesting that the cells engaged in nano4 directly (FIG. 1E). More than 95% of CD11c⁺SRB⁺ cells were identified as AMs (FIG. 10A, second panel) or 44% of total AMs in the lung took up the liposomes shown as SRB⁺DiD⁺ (FIG. 10B, first panel). The proportions of SRB⁺AMs and SRB⁺AECs peaked at 12 h and 18 h, respectively, returning to basal levels within 36 h (FIG. 8B). In marked contrast, very few pulmonary CD103⁺ dendritic cells (DCs) (<2%) and CD11b⁺ DCs (<2%) were DiD⁺ and SRB⁺, which ruled out the direct uptake of the liposomes by these cells (FIG. 10B). The ability of PS-GAMP to deliver cGAMP into AMs was functionally verified by CD40 upregulation in DiD⁺ AMs after i.n. inoculation with DiD-labeled and cGAMP-encapsulated nano4 (DiD-PS-GAMP). The same nanoparticle lacking cGAMP (DiD-PS) had no effect on CD40 expression (FIGS. 11A-11B) (19). Thus, AM activation appears to result directly from PS-GAMP uptake rather than through a bystander effect (FIG. 11C). In contrast with nano4, nano5 did not significantly associate with either AMs or AECs when compared with free SRB (FIGS. 1B-1D and FIG. 5C).

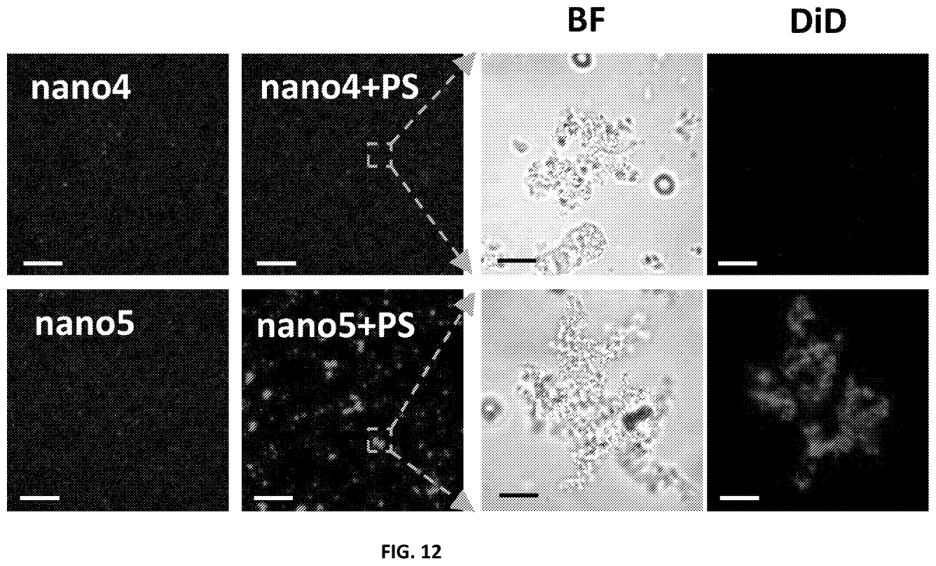

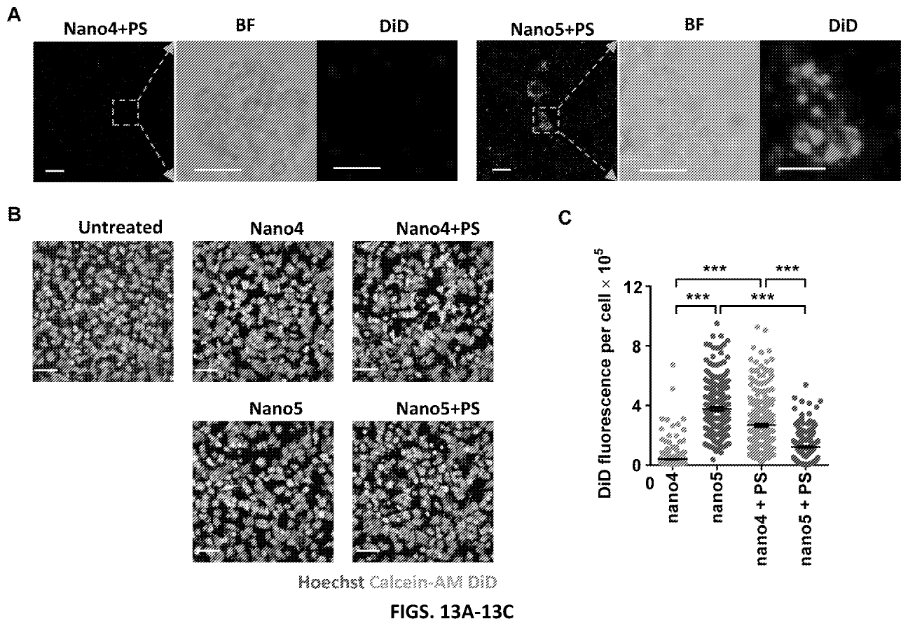

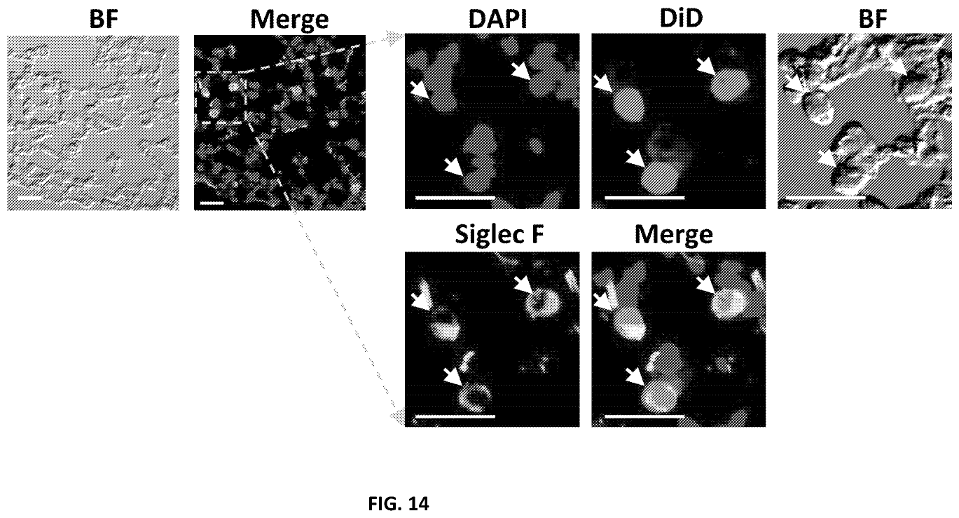

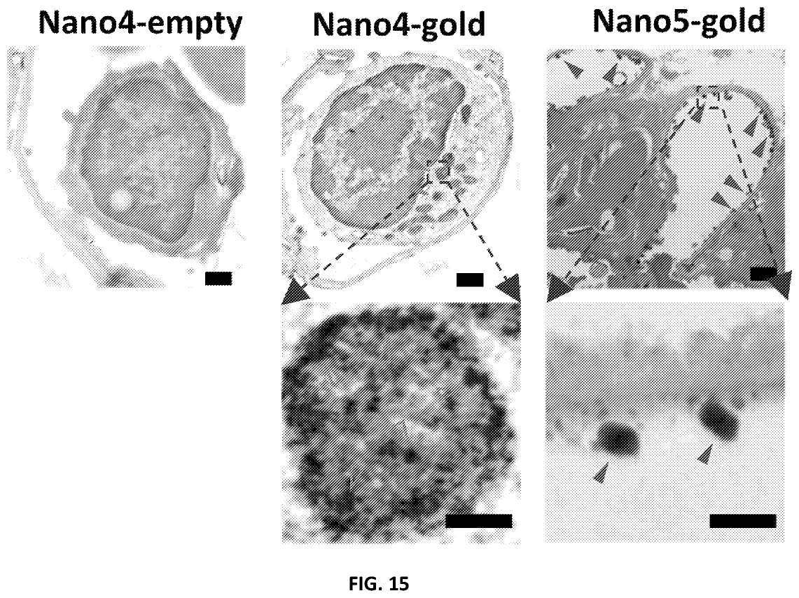

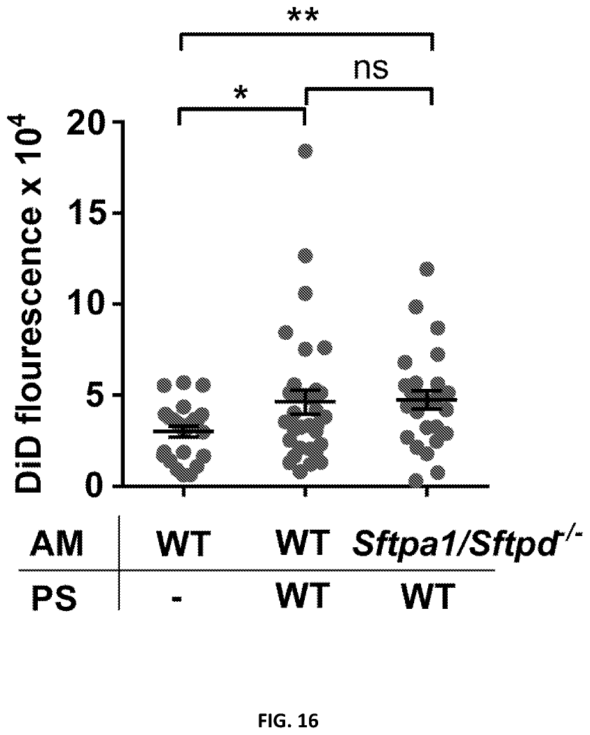

Surprisingly, AMs isolated from lung lavage did not efficiently ingest nano4 ex vivo. AMs in fact took up more nano5 than nano4 as evidenced by higher DiD fluorescence (FIGS. 1F-1G), which complemented our earlier observation that nano5 induced higher Ifnb1 expression in BMDCs and BMMs (FIGS. 7H-71). Differences between in vivo and ex vivo uptake of these liposomes may have been due to the lack of PS in ex vivo cultures. We therefore purified PS from BALF and incubated the PS with nanoparticles for 30 min before adding them to AMs. Nano4 uptake increased substantially, whereas nano5 uptake was diminished (FIGS. 1F-1G). Notably, positively charged nano5 aggregated on the negatively charged PS, explaining its poor entry of AMs (FIG. 12). No such aggregates were formed when PS was incubated with nano4 under similar conditions (FIG. 12). Similar results were obtained when AMs and PS were isolated from non-human primates (NHP) (FIGS. 13A-13C). Thus, PS may play an evolutionarily conserved role in PS-GAMP endocytosis. Consistent with these ex vivo observations, DiD-nano4 localized within individual cells positive for Siglec F, a biomarker for AMs following i.n. administration (FIG. 1H and FIG. 14). In contrast, positively charged nano5 electrostatically interacted and fused with negatively charged PS, exhibiting diffuse staining along the alveolar surface (FIG. 1H). Distinct localizations of nano4 and nano5 were corroborated by transmission electron microscopy (TEM) using nanogold-labeled nano5 and nano4 (FIG. 15). In vitro validation of effective uptake of nano4 only in the presence of PS hinted that surfactant proteins (SP)-A and -D (termed “collectins”) played a role in this uptake. Indeed, PS isolated from Sftpa1^{-/-}Sftpd^{-/-} mice failed to enhance nano4 uptake by WT AMs in vitro over controls, in marked contrast to PS isolated from WT mice (FIG. 1I). Moreover, nano4 uptake was severely impeded in Sftpa1^{-/-}Sftpd^{-/-} mice (FIG. 1J), which was not due to any defect of Sftpa1^{-/-}Sftpd^{-/-} AMs, since Sftpa1^{-/-}Sftpd^{-/-} AMs took up comparable amounts of nano4 as WT AMs did after pre-incubation in vitro with WT PS (FIG. 16).

### Example 3: PS-GAMP Transiently Activates Innate Immunity in the Lung

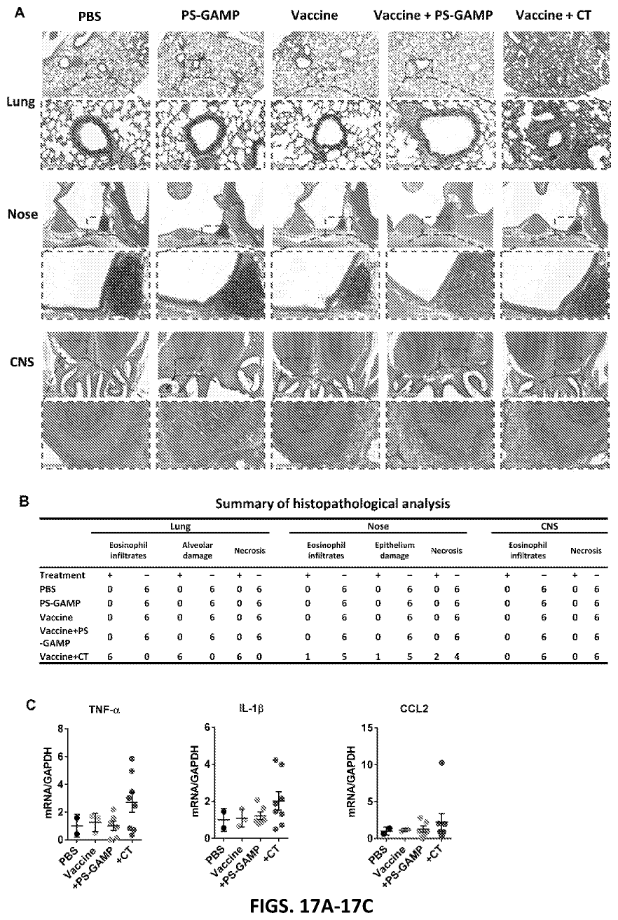

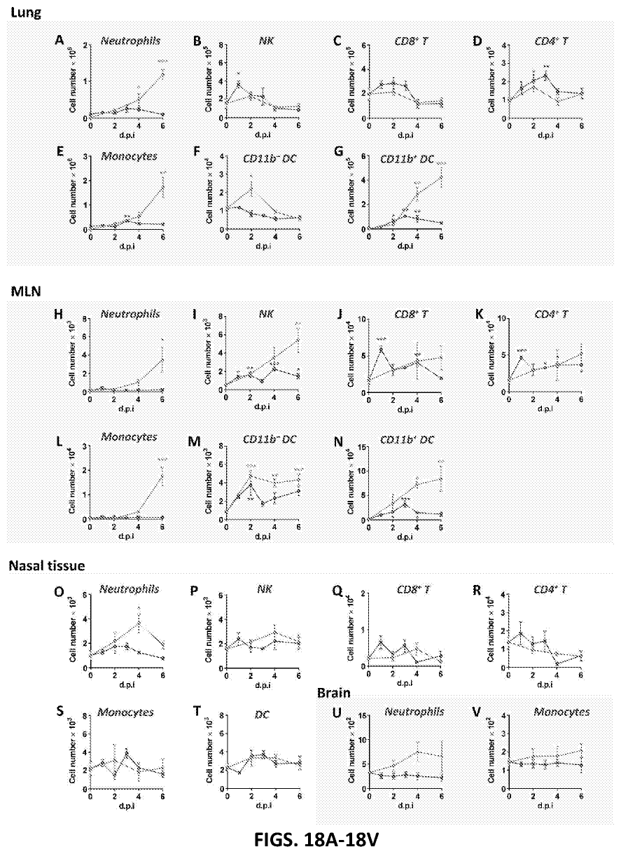

Reliance on SP-A and SP-D in nano4 uptake suggested that a natural and molecule-specific mechanism of particle clearance in the lung was involved, which would be the best approach to sustain the integrity of PS and alveolar epithelial barriers (20). Indeed, 2 d after PS-GAMP, whole inactivated VN04H5N1 vaccine, or a combination of both was i.n. administered, mouse lungs, nasal tissue, and brains were histologically indistinguishable from PBS controls (FIGS. 17A-17B). There was no cell death, damage to the epithelial barrier, or overt infiltration of inflammatory cells in these tissues (FIGS. 17A-17C). Only modest and transient infiltration of monocytes was found in the lung on day 3, which was substantially less severe than the monocyte response to viral infection (FIG. 18E). We also did not observe any significant cytokine production in the brain over controls (FIG. 17C). In sharp contrast, VN04 H5N1 vaccine formulated with cholera toxin (CT) provoked both massive inflammatory cell infiltrates in the lung and measurable cytokine mRNA expression in the brains of some mice (FIGS. 17A-17C).

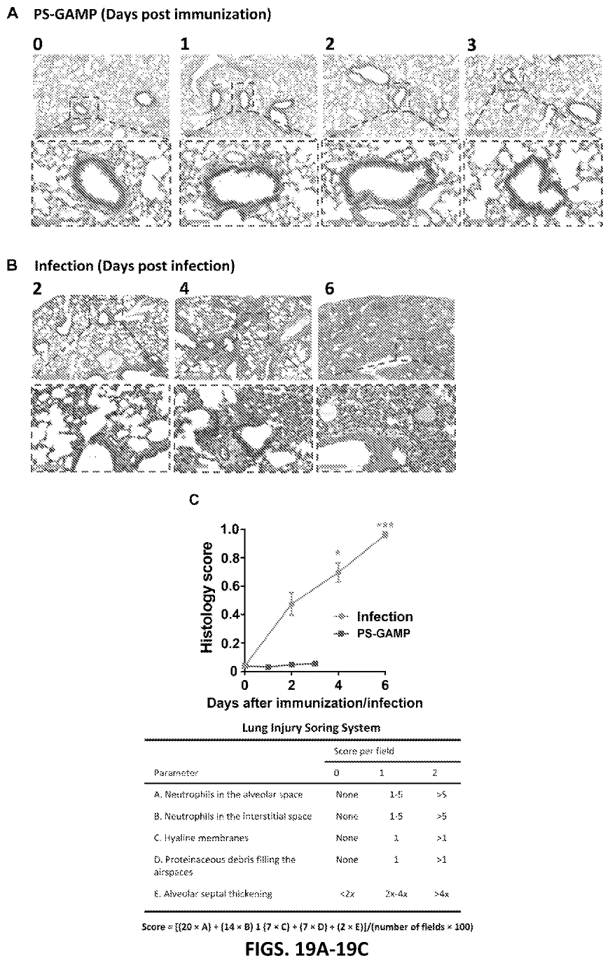

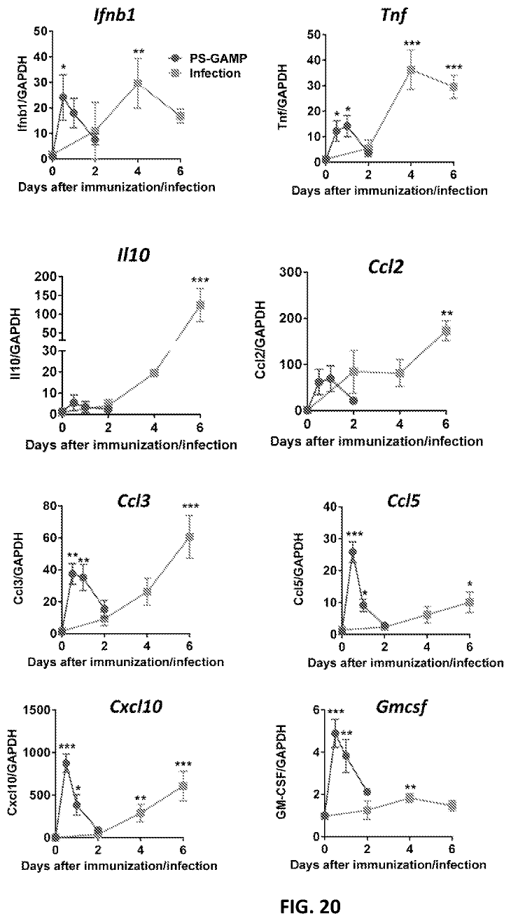

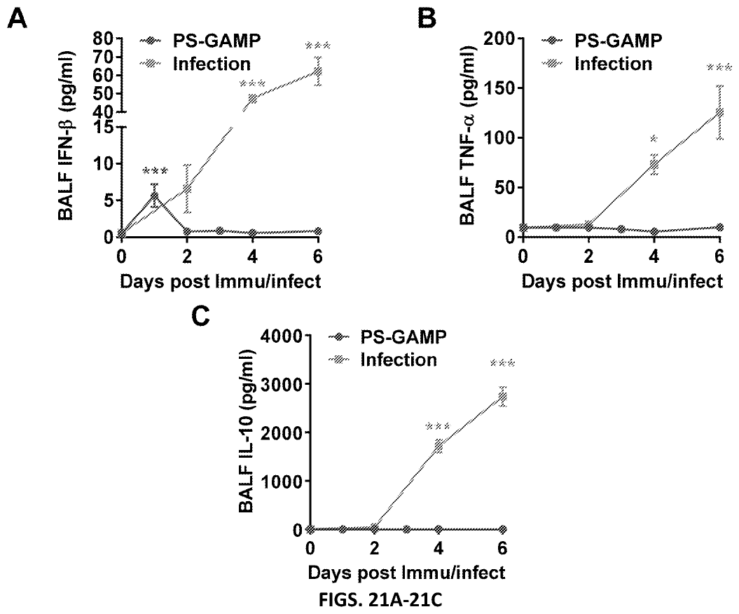

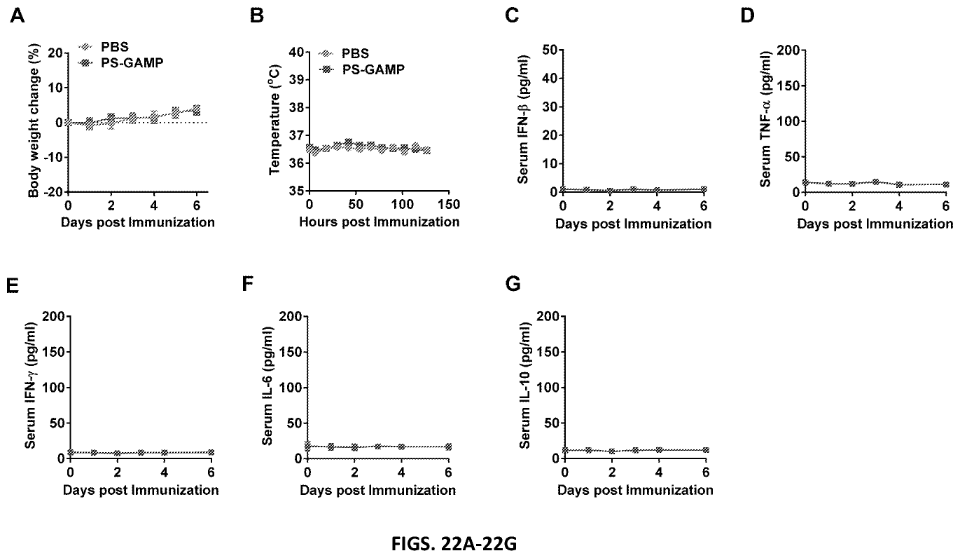

Despite the lack of overt lung inflammation over time histologically (FIG. 19A), PS-GAMP was found to rapidly and robustly, but only transiently, activate innate immunity. Ifnb1, Gmcsf, and Tnf as well as Ccl2, Ccl3, Ccl5, and Cxcl10 mRNA expressions peaked 12 h post-stimulation and resolved within 48 h (FIG. 20). By contrast, a low-dose infection with CA09 H1N1 influenza virus induced substantially higher levels of these mediators (FIG. 20), giving rise to overt lung inflammation that was worsening over the course of viral infection, despite robust I110 expression (FIGS. 19B-19C and 20). The transient IFN-β production was also corroborated at protein levels in BALF, but TNF-α and IL-10 were beyond the detection limit (FIGS. 21A-21C). In marked contrast, these cytokine were produced substantially higher from d 2 to 6 as infection proceeded (FIGS. 21A-21C). The transient activation of innate immunity was confined to the lung as serum IFN-β, IFN-γ, IL-6, IL-10, and TNF-α levels were unaltered compared to controls (FIGS. 22C-22G). This concurred with the lack of adjuvant side effects in terms of mouse body weight and temperature (FIGS. 22A-22B).

### Example 4: PS-GAMP is a Powerful Adjuvant for Both Humoral and Cellular Immune Responses

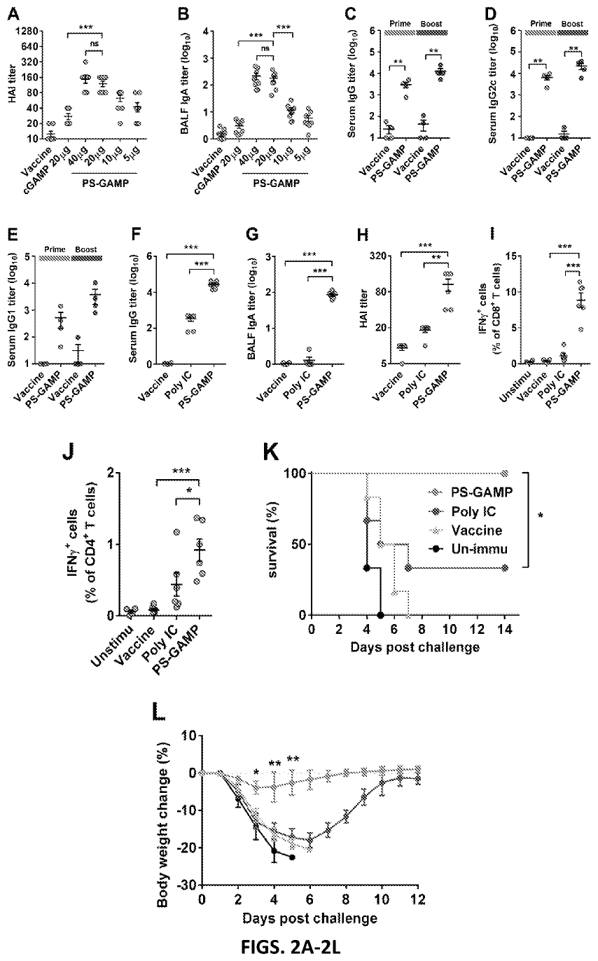

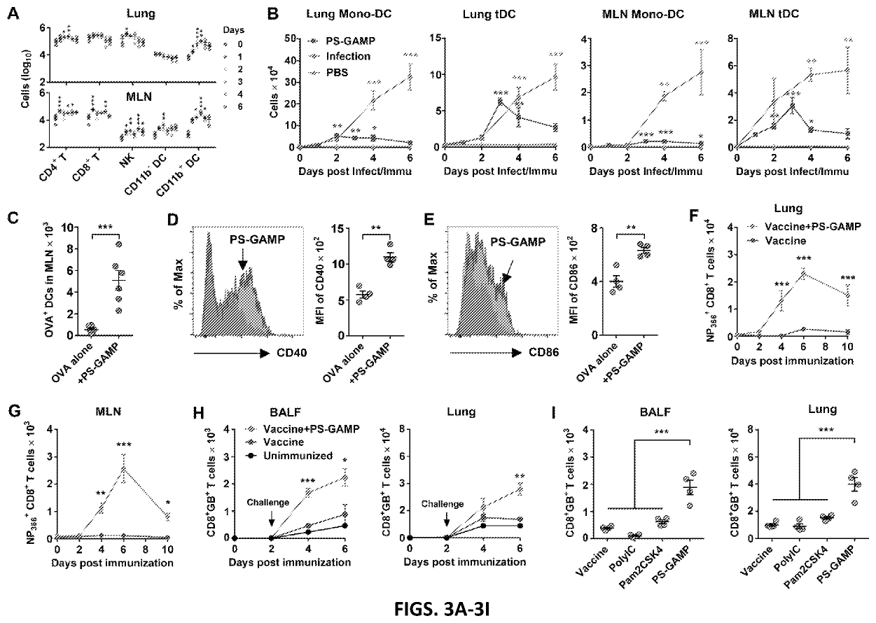

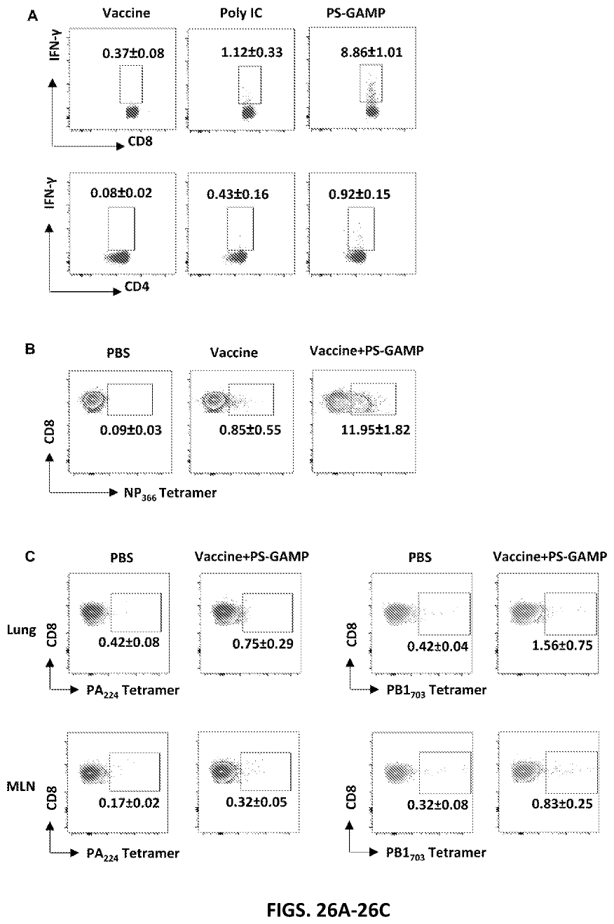

Although PS-GAMP only transiently activated innate immunity, this effect appeared to be sufficient to augment both humoral and cellular immune responses, consistent with our previous findings that prolonged activation of innate immunity was not necessary for strong adaptive immunity (13, 21, 22). PS-GAMP elevated serum hemagglutination inhibitory (HAI) antibody and BALF IgA titers in a dose-dependent manner (FIGS. 2A-2B). The adjuvant was potent in both primary and booster immune responses, raising Ag-specific IgG1 tenfold, IgG more than 100-fold, and IgG2c˜1,000-fold over VN04 H5N1 vaccine alone in the serum (FIGS. 2C-2E). In addition to the whole inactivated VN04 H5N1 vaccine, PS-GAMP also exhibited strong adjuvanticity when combined with split virion (SV) vaccines like the A/California/7/2009 (CA09) H1N1 vaccine. The adjuvant augmented HAI titers tenfold, BALF IgA 60-fold, and IgG 10,000-fold over the SV vaccine alone (FIGS. 2F-2H). Under similar conditions, poly IC showed fivefold, 30-fold, and 100-fold lower efficacy in augmenting HAI titers, BALF IgA, and serum IgG, respectively (FIGS. 2F-2H). PS-GAMP not only augmented humoral immune responses, but also profoundly enhanced cellular immune responses. PS-GAMP-adjuvanted CA09 H1N1 vaccine increased IFN-γ⁺CD8⁺ T cells 24-fold compared to vaccine alone or eightfold over the vaccine formulated with poly IC (FIG. 21 and FIG. 26A). The duo also induced the highest amount of IFN-γ⁺CD4⁺ T cells among all vaccination groups (FIG. 2J and FIG. 26A). The robust immune responses translated into full protection against 10×LD₅₀ CA09 H1N1 viral challenge, concurrent with only mild to no body weight loss (FIGS. 2K-2L). In contrast, poly IC-adjuvanted CA09H1N1 vaccine conferred only partial (33%) protection against the viral challenge with severe body weight loss.

### Example 5: PS-GAMP Elicits Robust CD8⁺ T Cell Responses

We interrogated which DC subsets were involved in PS-GAMP-mediated adjuvanticity and found that after i.n. administration of PS-GAMP, CD11b⁺ DCs, but not CD11b⁺ DCs, were elevated 14-fold and 36-fold on d 3 relative to d 0 in the lung (upper) and MLN (low), respectively (FIG. 3A). Among CD11b⁺ DCs, monocyte-derived CD11b⁺ DCs (Mono-DCs) and tissue-resident CD11b⁺ DCs (tDCs) were distinguished by MHC II and Ly6C expression (FIG. 3B) (23, 24). MHC II^{hi}CD11b⁺ tDCs have been shown to be the most competent lung DCs for cross-presentation during influenza viral infection (23). Remarkably, following PS-GAMP administration, these cells were vigorously accumulated resembling those in the early phase (the first 3 d) of viral infection, whereas pro-inflammatory mono-DCs were increased only slightly, albeit significantly relative to d 0, during the same experimental period (FIG. 3B). CD11b⁺ tDCs declined thereafter in the lung and MLN receiving PS-GAMP, in marked contrast to continuous accumulation of these CD11b⁺ DCs in both the lung and MLN beyond 3 d of infection (FIG. 3B). Changes of other immune cell types were detailed in the lungs, MLNs, nasal tissue, and brains after immunization or infection in FIGS. 18A-18V. In addition to DCs, natural killer (NK) cells and CD4⁺ T cells were briefly elevated for 1 or 2 d in the lung, while other immune cells were unaltered during the experimental period (FIG. 3A).

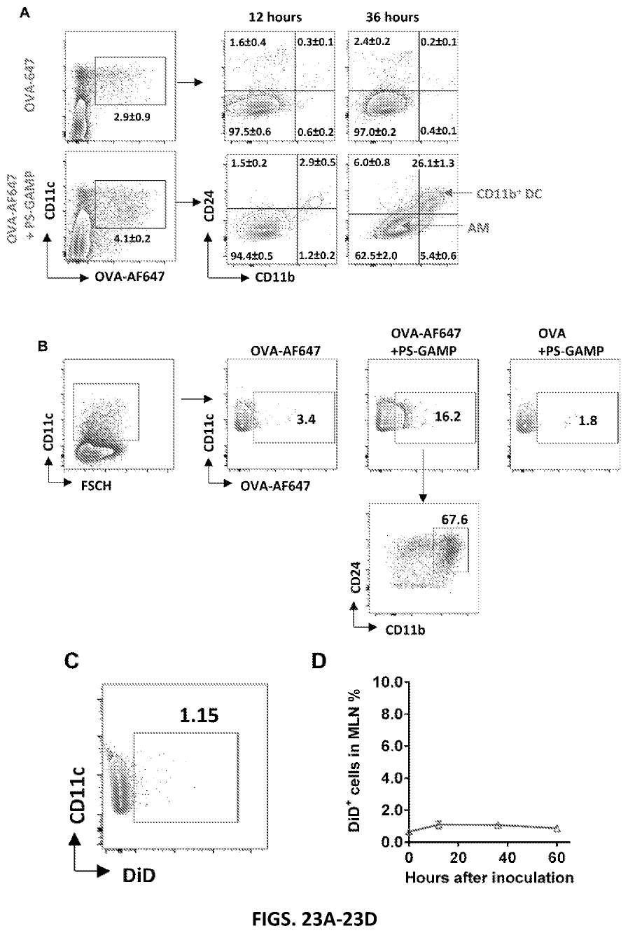

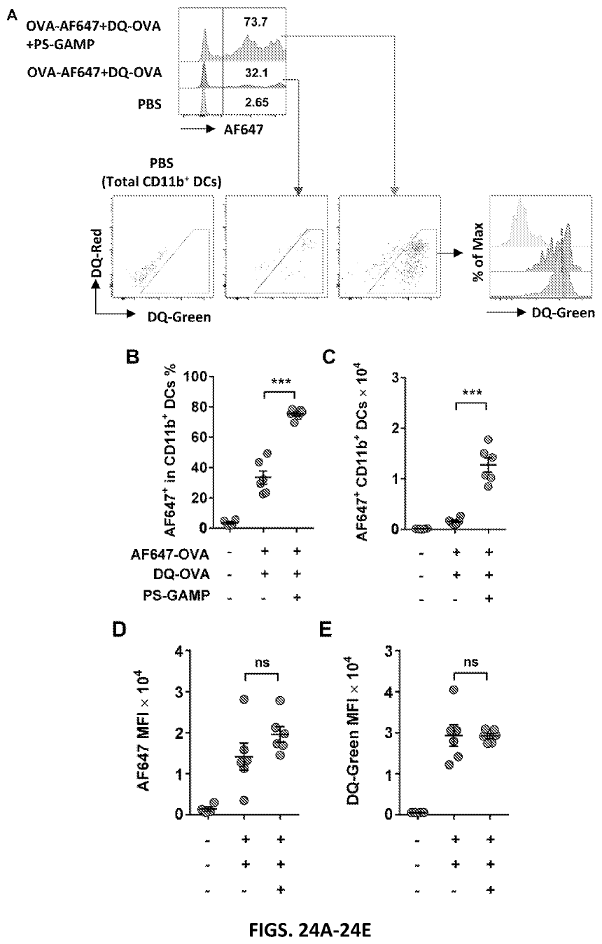

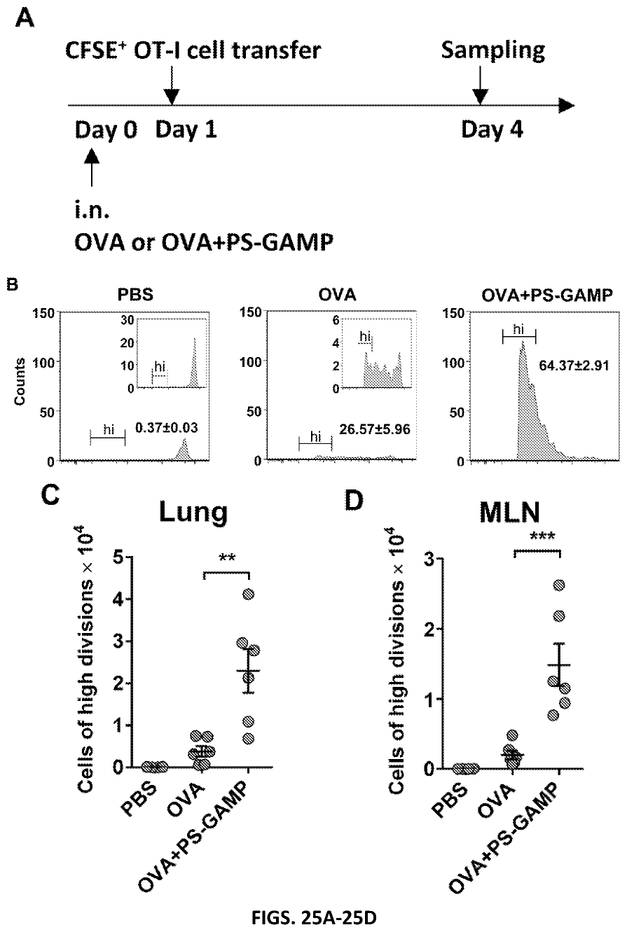

These CD11b⁺DCs appeared to be efficient at Ag cross-presentation and could induce robust CD8⁺ T cell proliferation. When fluorescently labeled ovalbumin (OVA) was i.n. administered, very few lung CD11b⁺ DCs (0.3%) showed OVA uptake. The proportion of these DCs ingesting OVA, however, rose substantially from 3% at 12 h to 26% at 36 h post-immunization in the presence of PS-GAMP (FIG. 23A). This translated to a tenfold increase in OVA⁺ DCs in the MLN with predominant CD11b⁺ DCs, compared with mice receiving OVA alone (FIG. 3C and FIG. 23B). These DCs had matured and were activated, as suggested by upregulation of CD40 and CD86 (FIGS. 3D-3E). This effect was presumably secondary to AEC and AM activation, considering that most MLN DCs were negative for PS-GAMP (FIGS. 23C-23D). The increase in Ag-specific CD11b⁺ DCs did not result from altered Ag-processing or Ag-uptake, because OVA uptake or its proteolytic cleavage was unaffected by PS-GAMP (FIGS. 24D-24E). Thus, the vigorous proliferation of OT-I cells in the presence of PS-GAMP was likely due to the augmented differentiation and maturation of CD11b⁺ DCs. These cells, in turn, gave rise to an over sixfold increase in highly proliferating OT-I cells in both the lungs and MLNs when OVA was introduced with PS-GAMP compared to OVA alone (FIGS. 25A-25D).

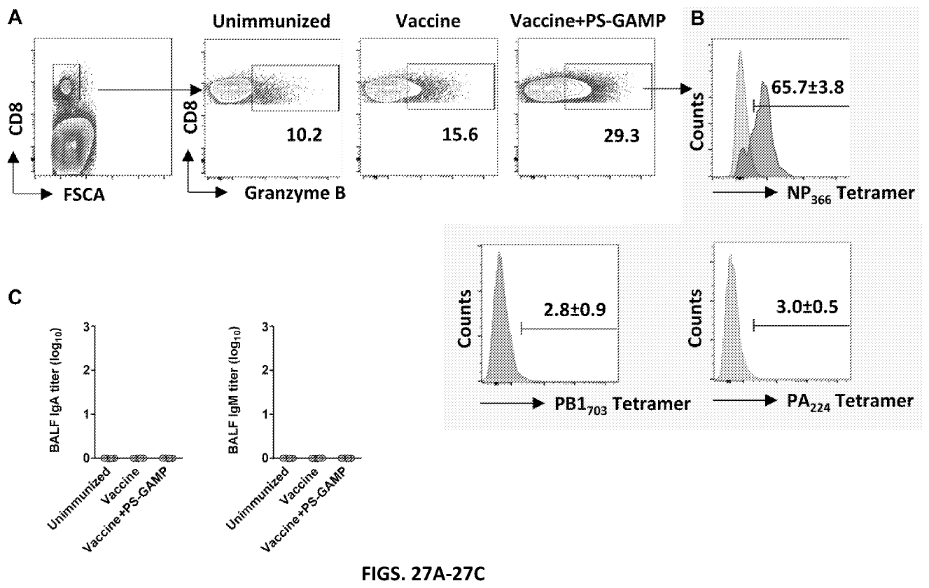

A large number of nucleoprotein (NP)₃₆₆₋₃₇₄-specific CD8⁺ T cells were observed in the lung and to a lesser extent, in the MLN, as early as 4 d after immunization with PS-GAMP-adjuvanted influenza vaccine (FIGS. 3F-3G and FIG. 26B). NP₃₆₆₋₃₇₄ was the dominant CD8⁺ T cell epitope and CD8⁺ T cells specific for other epitopes, such as PA₂₂₄₋₂₃₃ or PB1₇₀₃₋₇₁₁. were undetectable in these animals, probably due to a low copy number of these proteins in inactivated influenza vaccine (FIG. 26C) (25). These virus-specific CD8⁺ T cells expressed the early activation biomarker, granzyme B (GB), upon viral challenge (FIG. 27A) (26). GB⁺CD8⁺ T cells rose significantly 4 d in BALF and 6 d in the lung after receiving PS-GAMP-adjuvanted CA09 H1N1 vaccine (FIG. 3H). More than 65% of these GB⁺CD8⁺ T cells were positive for NP₃₆₆₋₃₇₄, whereas only a few cells were positive for PA₂₂₄₋₂₃₃ or PB1₇₀₃₋₇₁₁ (FIG. 27B). Under similar conditions, the vaccine alone failed to expand GB⁺CD8 T cells significantly (FIG. 3H). The CD8⁺ T cell response evoked by PS-GAMP was found to be superior to poly IC or TLR2 agonist Pam2CSK4 (27, 28) (FIG. 3I). Although T cell immune responses were induced soon after immunization, Ag-specific BALF IgA and IgM were undetectable at these early time points (FIG. 27C). Thus, PS-GAMP mimics the crucial events of viral infection in terms of CD8⁺ T cell induction without provoking excessive lung inflammation or immunopathology (FIGS. 17A-17C, 18A-18V, 19A-19C, 20, 21A-21C, and 22A-22G).

### Example 6: PS-GAMP Offers Robust Protection as Early as 2 d Post-Immunization

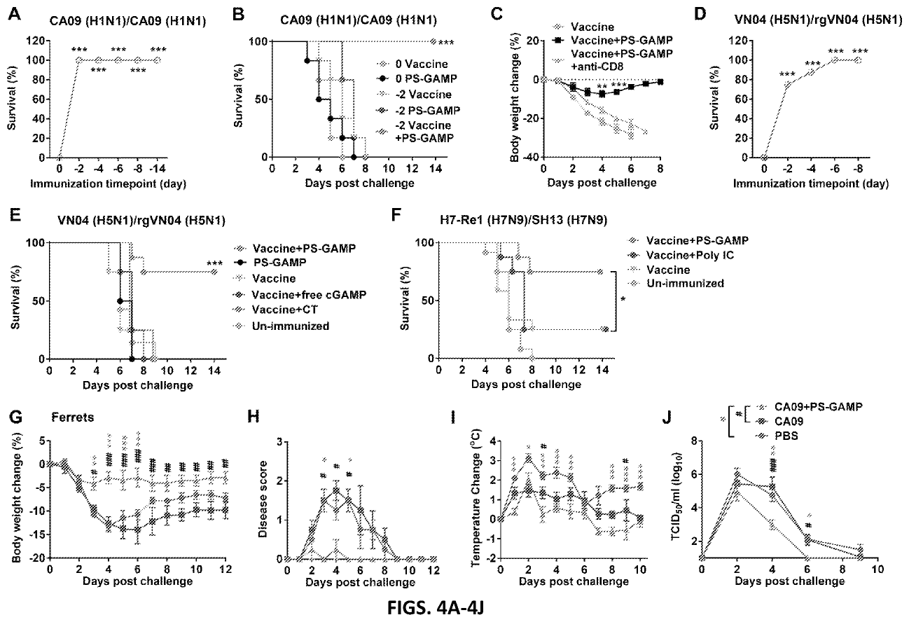

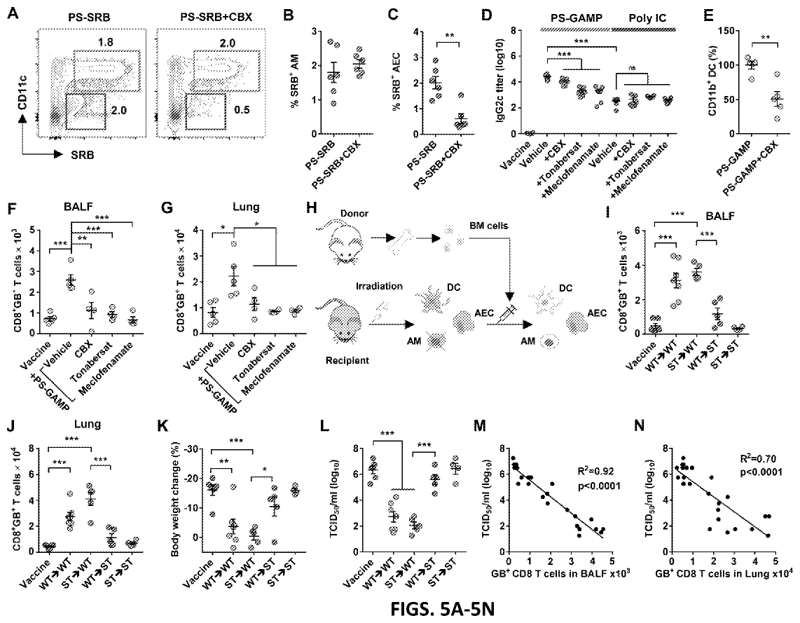

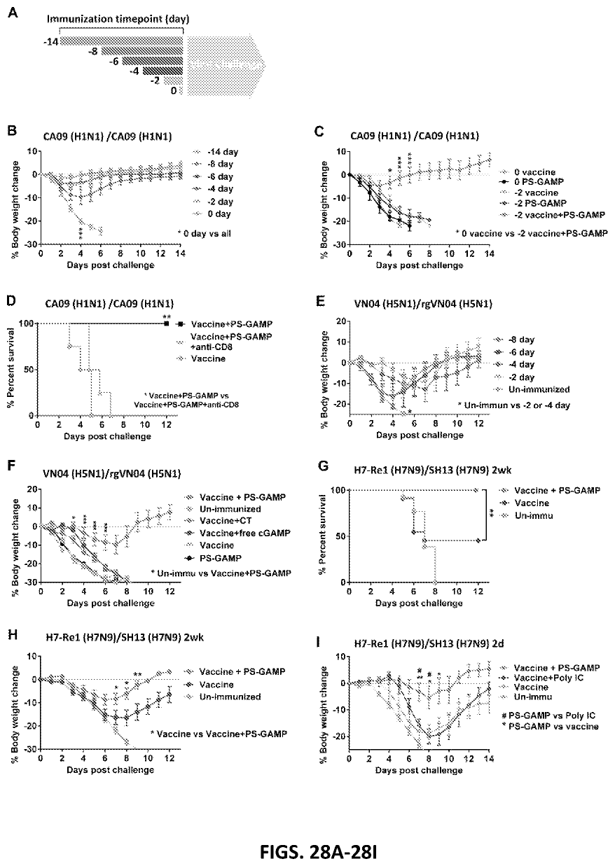

The rapid induction of CD8⁺ T cells prompted us to determine how quickly protection could be achieved by PS-GAMP. To this end, mice were challenged on day 0, 2, 4, 6, 8, or 14 after immunization as depicted in FIG. 28A. Inclusion of PS-GAMP in the vaccination fully protected mice from homologous viral challenges as early as 2 d post-immunization (FIG. 4A). At all early challenge timepoints (d −2, −4, and −6), mice experienced only slight body weight loss (<10%) and all mice survived (FIG. 4A and FIG. 28B). When challenged 8 d post-immunization, the mice did not suffer from any body weight loss with 100% survival (FIG. 4A and FIG. 28B). This early protection did not result directly from innate immunity, as PS-GAMP alone given on day 0 or 2 prior did not confer any protection (FIG. 4B and FIG. 28C). To determine whether CD8⁺ T cells were responsible for the early protection, CD8⁺ T cells were depleted by intraperitoneal (i.p.) injections of anti-CD8 antibody every other d starting 2 d prior and ending 4 d post-immunization. Depletion of CD8⁺ T cells abolished the early protection, as evidenced by a precipitous body weight loss and 100% mortality similar to those of mice receiving vaccine alone (FIG. 4C and FIG. 28D). To preclude that this early protection was unique for CA09 H1N1 vaccine, we extended the investigation to H5N1 vaccine, which is an immunogenically weak vaccine compared with CA09 H1N1 vaccines. Once again, the presence of PS-GAMP conferred 75%-100% protection against rgVN04 H5N1 viral challenge for mice that had been immunized 2-8 d prior in a manner dependent on adaptive immune responses (FIG. 4D and FIG. 28E), because no protection was attained with PS-GAMP alone (FIG. 4E and FIG. 28F). Under similar conditions, the vaccine combined with CT did not provide any early protection (FIG. 4E and FIG. 28F), arguing persuasively that the heightened inflammation is not necessarily associated with strong adaptive immune responses (FIGS. 17A-17C). Besides rgH5N1 virus, mice were significantly or fully protected from a lethal challenge of a clinical isolate of pre-pandemic A/Shanghai/4664T/2013 (SH13) H7N9 virus 2 or 14 d after immunization with PS-GAMP-adjuvanted inactivated H7N9 vaccine (H7-Rel) (FIG. 4F and FIGS. 28G-281). The vaccine adjuvanted by poly IC did not provide any benefit over the vaccine alone under similar conditions (FIG. 4F and FIG. 281).

The ability of PS-GAMP to quickly establish protection was also validated in an FDA-approved ferret model. Ferrets receiving PS-GAMP-adjuvanted CA09 H1N1 vaccine 2 d prior experienced <5% body weight loss when infected with homologous CA09H1N1 virus, concomitant with mild to no clinical symptoms, and only a brief fever on d 2 following viral challenges (FIGS. 4G-4I). The virus shedding was significantly blunted from d 4 and beyond (FIG. 4J). However, CA09H1N1 vaccine alone failed to prevent the animals from body weight loss following similar viral challenge and did not improve clinical symptoms or reduce viral shedding over controls, despite moderately lowering body temperature (FIGS. 4G-4J).

### Example 7: AEC are Indispensable for PS-GAMP-Mediated Adjuvanticity

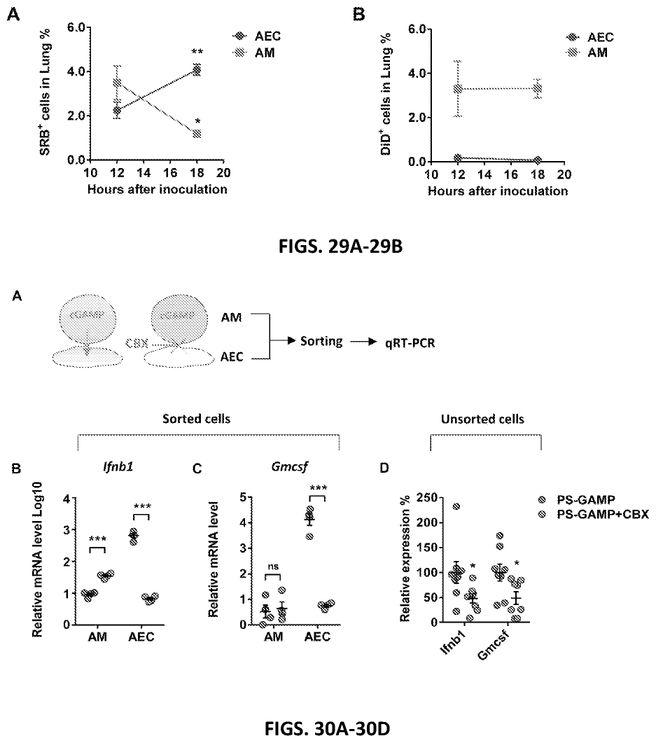

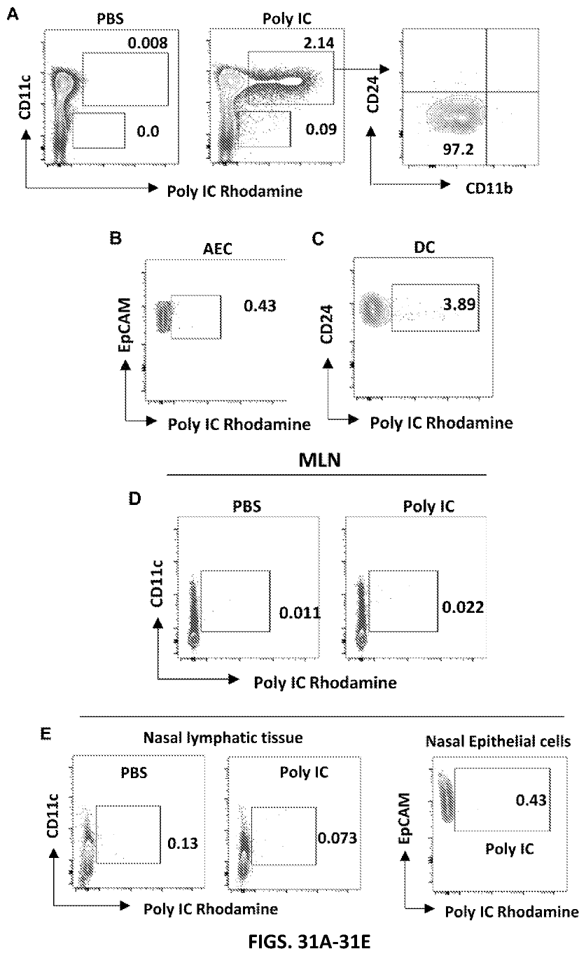

cGAMP is well documented as readily transferred via gap junctions presented between AMs and AECs (29, 30). A dynamic flux from AMs to AECs was demonstrated by the gradual loss of SRB in AMs, concurrent with the continuous gain of SRB in AECs from 12 h to 18 h after i.n. administration of SRB-nano4 (FIG. 29A). The loss of SRB in AMs could not be ascribed to a loss of the liposomes, since the number of DiD⁺ cells was unaltered up to 18 h later (FIG. 29B). The entry of SRB into AECs was blocked by carbenoxolone (CBX) (FIGS. 5A and 5C), a gap-junction blocker (29), which did not affect SRB uptake by AMs (FIGS. 5A-5B). In AMs and AECs sorted from lungs receiving PS-GAMP, CBX greatly diminished the transcription of Ifnb1 and Gmcsf in AECs, while increasing Ifnb1 transcription in AMs (FIGS. 30B-30C). This was most likely a consequence of elevated cGAMP levels in the cells. Thus, there is a gap-junction-mediated flux of cGAMP to AECs from AMs. Poly IC, on the other hand, remained primarily within AMs (>97%) after i.n. immunization (FIG. 31A). Only 0.4% of total AECs and 4% of total DCs took up poly IC in the lung (FIGS. 31B-31C). MIN and nasal tissue DCs as well as nasal epithelial cells rarely internalized poly IC (FIGS. 31D-31E).

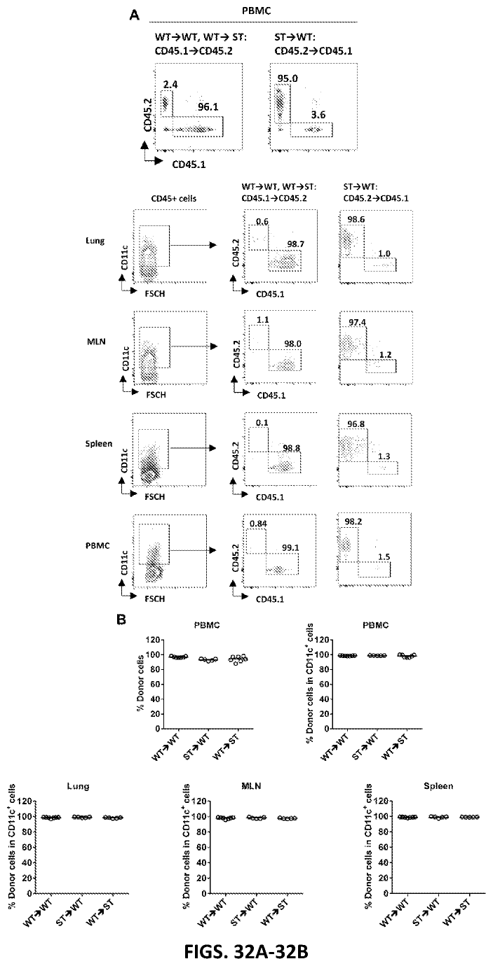

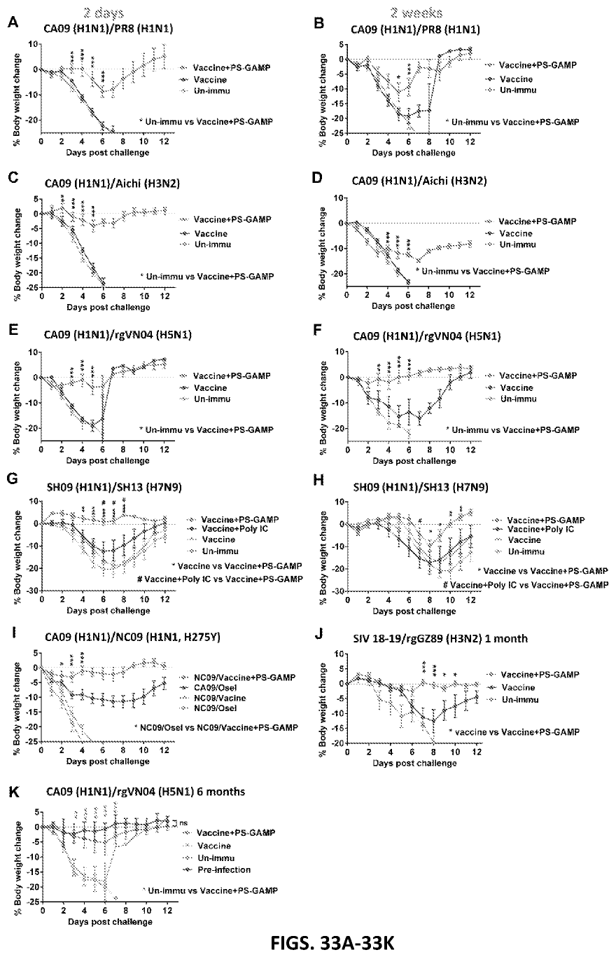

PS-GAMP induced 100-fold higher IgG2c titers than poly IC (FIG. 5D). The adjuvanticity was however blunted substantially when mice were treated with CBX or two other gap junction inhibitors tonabersat and meclofenamate prior to and during immunization (FIG. 5D) (31, 32). In contrast, these inhibitors had few effects on poly IC-mediated adjuvanticity (FIG. 51)), consistent with the inability of poly IC, a large molecule, to enter the neighbor cells via gap junctions (FIG. 31A). The blockade on the entry of cGAMP into AECs reduced recruitment of CD11b⁺ DCs by 50% (FIG. 5E) and exhibited more profound effects on the early CD8⁺ T cell responses in both the BALF and lung (FIGS. 5F-5G). Moreover, chimeric mice (ST→WT) comprising Sting-deficient (Sting^{-/-} or ST) bone marrow (BM) cells and WT AECs had similar levels of CD8⁺ T cells as WT→WT mice in both BALF and lung (FIGS. 5H-5J and FIGS. 32A-32B) (33). In contrast, mice with STING-deficiency in AECs prepared by transferring WT BM cells into Sting-deficient mice (WT→ST mice), generated significantly lower levels of Ag-specific CD8⁺ T cells (FIGS. 5I-5J). These WT→ST mice showed poor protection by PS-GAMP-adjuvanted CA09 H1N1 vaccine, as suggested by body weight loss and high lung viral titers, in contrast to the similarly observed protection between ST→WT and WT→WT mice (FIGS. 5K-5L). We also found an inverse correlation between the number of GB⁺CD8⁺ T cells in BALF and lung with viral titers, further supporting the pivotal role of GB⁺CD8⁺ T cells in control of the infections (FIGS. 5M-5N). Thus, AECs rather than AMs appear to be essential for determining the potency of PS-GAMP, consistent with their pivotal role in orchestrating innate and adaptive immune responses in the respiratory system during viral infection (24, 34-36).

### Example 8: PS-GAMP Broadens Protection Against Heterosubtypic Influenza Viruses

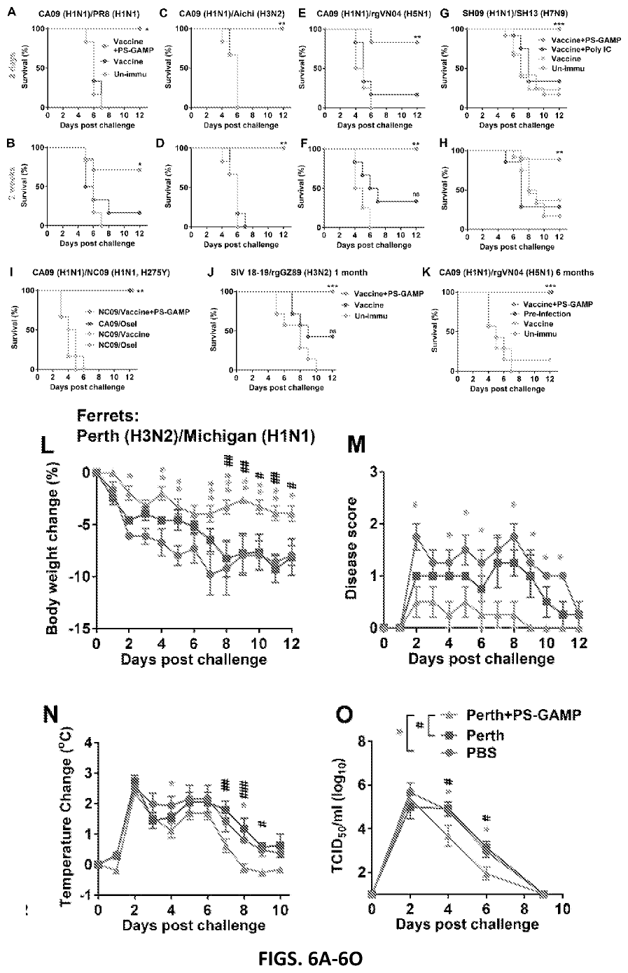

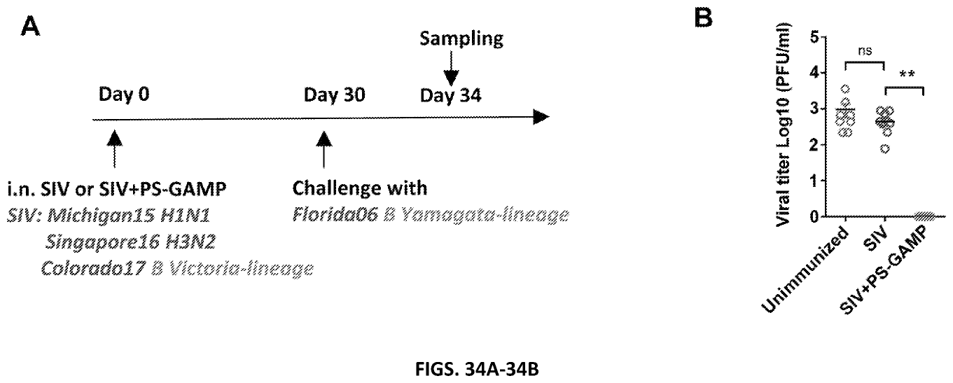

The robust CD8⁺ T cell immunity provoked by PS-GAMP permitted us to study its role in heterosubtypic protection, an issue of intense debate in the universal influenza vaccine field. Mice receiving CA09H1N1 vaccine (FIGS. 6A-6F) or A/Shanghai/37T/2009 (SH09) H1N1 vaccine (FIGS. 6G-6H), together with PS-GAMP, were highly protected from lethal challenges with distinct PR8 H1N1 virus and heterosubtypic A/Aichi/2/1968 (Aichi) H3N2, rgVN04 H5N1 or the highly pathogenic SH13 H7N9 virus, irrespective of whether the animals were infected at either 2 d (FIGS. 6A, 6C, 6E, and 6G) or 14 d (FIGS. 6B, 6D, 6F, and 6H) post-immunization (FIGS. 33A-33H). The vaccination also protected against an oseltamivir-resistant A/North Carolina/39/2009 H1N1 virus with an H275Y mutation (NC09) (FIG. 61 and FIG. 331), which emerged during 2009 H1N1 pandemic and H7N9 epidemic (37, 38). The resistance of this virus to oseltamivir was verified by the ability of oseltamivir to effectively control CA09H1N1, but not NC09, viral infection (FIG. 61). Under similar conditions, the H1N1 vaccines alone provided no or low protection against the challenges of these heterosubtypic variants (FIGS. 6A-6I and FIGS. 33A-331). In contrast to PS-GAMP, poly IC-adjuvanted SH09 H1N1 vaccine failed to provoke significant heterosubtypic protection against H7N9 virus (FIGS. 6G-6H and FIGS. 33G-33H). In addition to monovalent vaccines, PS-GAMP enhanced the breadth of immune responses induced by trivalent 2018-2019 seasonal influenza vaccines (SIV18-19) against mismatched reassortant A/Guizhou/54/1989 H3N2 (rgGZ89) virus (FIG. 6J and FIG. 33J) or Florida/4/2006 influenza B virus from Yamagata-lineage (FIGS. 34A-34B). These findings suggest that PS-GAMP can simultaneously augment multiple influenza vaccines and is similarly effective for both influenza A and B viral vaccines.

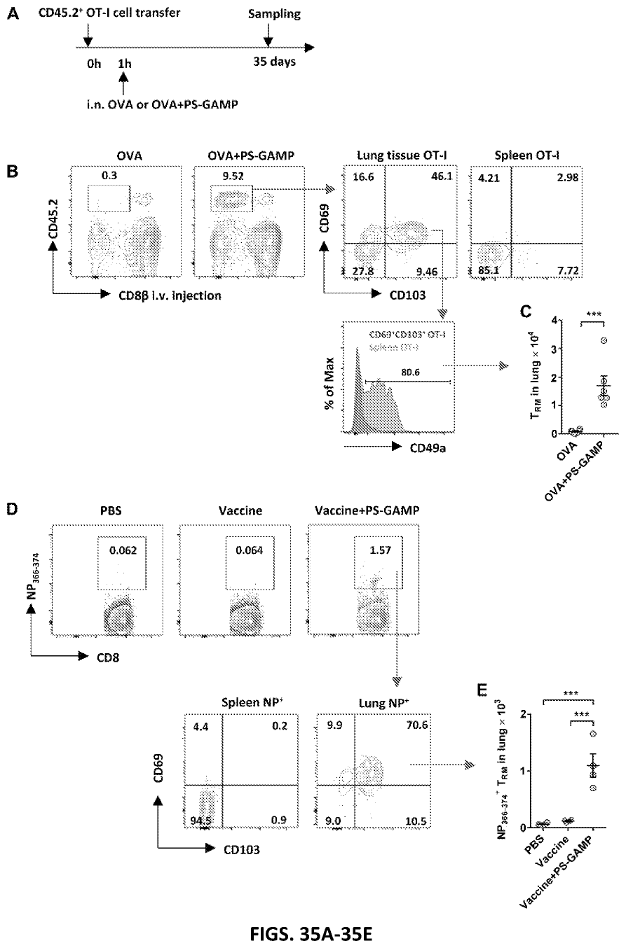

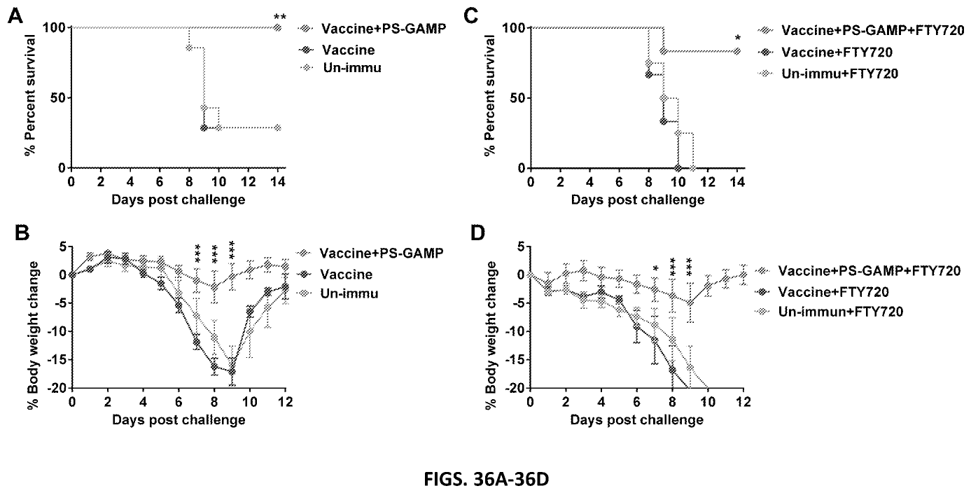

Long-lived, Ag-specific memory CD8⁺ T cells capable of rapid recall upon viral infection are pivotal for sufficient control of viral replication in the lung (2, 3). In mice receiving OT-I cells, the number of lung CD8⁺ T_{RM} cells, as marked by CD103⁺CD49a⁺CD69⁺, rose 20-fold after immunization with OVA combined with PS-GAMP relative to OVA alone (FIGS. 35A-35C). Moreover, PS-GAMP-adjuvanted CA09 H1N1 vaccine fully protected mice from heterosubtypic rgVN04 H5N1 viral challenge 6 months after a single immunization (FIG. 6K and FIG. 33K). This long-term cross-protection concurred with durable influenza-specific CD8⁺ T_{RM} cells in the lung, which could be readily detected in 6 months post-immunization (FIGS. 35D-35E). These CD8⁺ T_{RM} cells, rather than circulating memory CD8⁺ T cells, contributed to the long-term protection observed because their function was not compromised by T cell egress inhibitor FTY720 (FIGS. 36A-36D) (3).

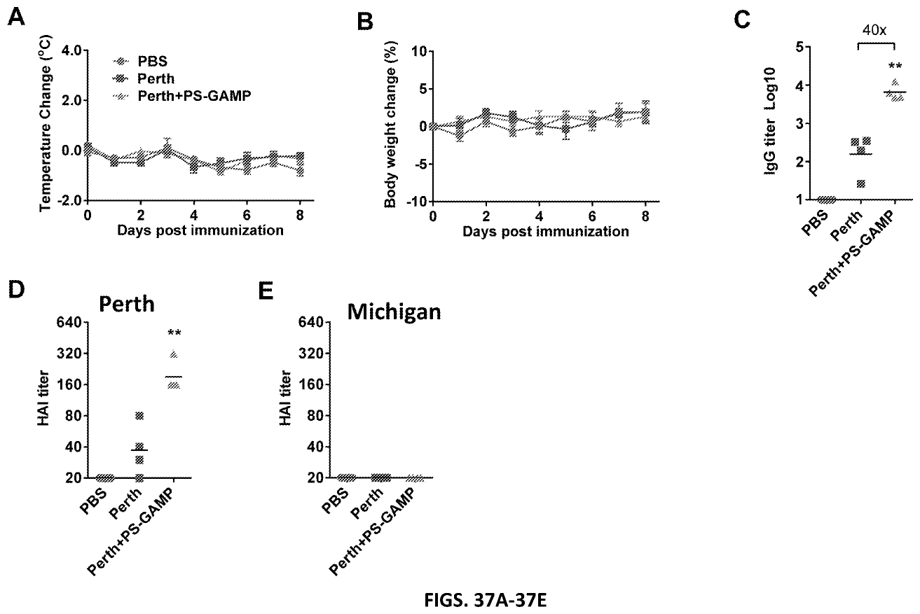

The heterosubtypic immunity was further corroborated in ferrets by immunization with PS-GAMP alongside inactivated rgPerthH3N2 vaccine. Body weight and temperature of the animals were not affected by the immunization when compared with those receiving PBS or the vaccine alone, demonstrating a good safety profile for PS-GAMP in ferrets (FIGS. 37A-37B). The immunization induced 40-fold higher serum IgG titers and fivefold higher HAI titers against homologous Perth H3N2 virus than vaccine alone did 28 d post-immunization (FIGS. 37C-37D), but no HAI antibody was detected against heterosubtypic A/Michigan/45/2015 H1N1 (Michigan H1N1) virus, as anticipated (FIG. 37E). Upon challenging with Michigan H1N1 virus, ferrets receiving the vaccine and PS-GAMP showed significantly less body weight loss and milder clinical symptoms, especially in the late phase (>7 d) of the infection, and normalized their temperature much faster than animals receiving PBS or vaccine alone (FIGS. 6L-6N). The animals also shed significantly a lower amount of virus after 2 d of infection (FIG. 6O). The ability of the vaccination to suppress viral replication resulting in significant improvement of clinical outcomes and acceleration of body weight recovery was likely a result of predominant T cell immunity in the animals.

### REFERENCES

1. C. I. Paules, S. G. Sullivan, K. Subbarao, A. S. Fauci, Chasing Seasonal Influenza—The Need for a Universal Influenza Vaccine. N. Engl. J. Med. 378, 7-9 (2018).
2. A. Pizzolla et al., Influenza-specific lung-resident memory T cells are proliferative and polyfunctional and maintain diverse TCR profiles. J. Clin. Invest. 128, 721-733 (2018).
3. K. D. Zens, J. K. Chen, D. L. Farber, Vaccine-generated lung tissue-resident memory T cells provide heterosubtypic protection to influenza infection. JCI Insight 1, (2016).
4. S. Sridhar et al., Cellular immune correlates of protection against symptomatic pandemic influenza. Nat. Med. 19, 1305-1312 (2013).
5. J. T. Weinfurter et al., Cross-reactive T cells are involved in rapid clearance of 2009 pandemic H1N1 influenza virus in nonhuman primates. PLoS Pathog. 7, e1002381 (2011).
6. M. Koutsakos et al., Human CD8(+) T cell cross-reactivity across influenza A, B and C viruses. Nat. Immunol. 20, 613-625 (2019).
7. L. Si et al., Generation of influenza A viruses as live but replication-incompetent virus vaccines. Science 354, 1170-1173 (2016).
8. L. Wang et al., Generation of a Live Attenuated Influenza Vaccine that Elicits Broad Protection in Mice and Ferrets. Cell Host Microbe 21, 334-343 (2017).
9. D. F. Hoft et al., Live and inactivated influenza vaccines induce similar humoral responses, but only live vaccines induce diverse T-cell responses in young children. J. Infect. Dis. 204, 845-853 (2011).
10. A. Ablasser et al., cGAS produces a 2′-5′-linked cyclic dinucleotide second messenger that activates STING. Nature 498, 380-384 (2013).
11. J. Wu et al., Cyclic GMP-AMP is an endogenous second messenger in innate immune signaling by cytosolic DNA. Science 339, 826-830 (2013).
12. X. D. Li et al., Pivotal roles of cGAS-cGAMP signaling in antiviral defense and immune adjuvant effects. Science 341, 1390-1394 (2013).
13. J. Wang, P. Li, M. X. Wu, Natural STING Agonist as an “Ideal” Adjuvant for Cutaneous Vaccination. J. Invest. Dermatol. 136, 2183-2191 (2016).
14. L. Corrales et al., Direct Activation of STING in the Tumor Microenvironment Leads to Potent and Systemic Tumor Regression and Immunity. Cell Rep. 11, 1018-1030 (2015).
15. J. A. Whitsett, S. E. Wert, T. E. Weaver, Alveolar surfactant homeostasis and the pathogenesis of pulmonary disease. Annu. Rev. Med. 61, 105-119 (2010).
16. K. A. Woodrow, K. M. Bennett, D. D. Lo, Mucosal vaccine design and delivery. Annu. Rev. Biomed. Eng. 14, 17-46 (2012).
17. E. Parra, J. Perez-Gil, Composition, structure and mechanical properties define performance of pulmonary surfactant membranes and films. Chem. Phys. Lipids 185, 153-175 (2015).
18. A. V. Misharin, L. Morales-Nebreda, G. M. Mutlu, G. R Budinger, H. Perlman, Flow cytometric analysis of macrophages and dendritic cell subsets in the mouse lung. Am. J. Respir. Cell Mol. Biol. 49, 503-510 (2013).
19. H. Qin, C. A. Wilson, S. J. Lee, X. Zhao, E. N. Benveniste, LPS induces CD40 gene expression through the activation of NF-kappaB and STAT-lalpha in macrophages and microglia. Blood 106, 3114-3122 (2005).
20. A. Haczku, Protective role of the lung collectins surfactant protein A and surfactant protein D in airway inflammation. J. Allergy Clin. Immunol. 122, 861-879; quiz 880-861 (2008).
21. J. Wang, B. Li, M. X. Wu, Effective and lesion-free cutaneous influenza vaccination. Proc. Natl. Acad. Sci. U.S.A 112, 5005-5010 (2015).
22. J. Wang, D. Shah, X. Chen, R R Anderson, M. X. Wu, A micro-sterile inflammation array as an adjuvant for influenza vaccines. Nat. commun. 5, 4447 (2014).
23. A. Ballesteros-Tato, B. Leon, F. E. Lund, T. D. Randall, Temporal changes in dendritic cell subsets, cross-priming and costimulation via CD70 control CD8(+) T cell responses to influenza. Nat. Immunol. 11, 216-224 (2010).
24. B. Unkel et al., Alveolar epithelial cells orchestrate DC function in murine viral pneumonia. J. Clin. Invest. 122, 3652-3664 (2012).
25. J. S. Sullivan et al., Heterosubtypic anti-avian H5N1 influenza antibodies in intravenous immunoglobulins from globally separate populations protect against H5N1 infection in cell culture. J. Mol. Genet. Med. 3, 217-224 (2009).
26. C. W. Lawrence, R M. Ream, T. J. Braciale, Frequency, specificity, and sites of expansion of CD8+ T cells during primary pulmonary influenza virus infection. J. Immunol. 174, 5332-5340 (2005).
27. T. Ichinohe et al., Synthetic double-stranded RNA poly(I:C) combined with mucosal vaccine protects against influenza virus infection. J. Virol. 79, 2910-2919 (2005).
28. J. Y. Kang et al., Recognition of lipopeptide patterns by Toll-like receptor 2-Toll-like receptor 6 heterodimer. Immunity 31, 873-884 (2009).
29. A. Ablasser et al., Cell intrinsic immunity spreads to bystander cells via the intercellular transfer of cGAMP. Nature 503, 530-534 (2013).
30. K. Westphalen et al., Sessile alveolar macrophages communicate with alveolar epithelium to modulate immunity. Nature 506, 503-506 (2014).
31. P. W. Furlow et al., Mechanosensitive pannexin-1 channels mediate microvascular metastatic cell survival. Nat. Cell Biol. 17, 943-952 (2015).
32. Q. Chen et al., Carcinoma-astrocyte gap junctions promote brain metastasis by cGAMP transfer. Nature 533, 493498 (2016).
33. H. Ramsey et al., Stress-induced hematopoietic failure in the absence of immediate early response gene X-1 (IEX-1, IER3). Haematologica 99, 282-291 (2014).
34. J. H. Fritz, L. Le Bourhis, J. G. Magalhaes, D. J. Philpott, Innate immune recognition at the epithelial barrier drives adaptive immunity: APCs take the back seat. Trends Immunol. 29, 41-49 (2008).
35. S. A. Saenz, B. C. Taylor, D. Artis, Welcome to the neighborhood: epithelial cell-derived cytokines license innate and adaptive immune responses at mucosal sites. Immunol. Rev. 226, 172-190 (2008).
36. J. A. Whitsett, T. Alenghat, Respiratory epithelial cells orchestrate pulmonary innate immunity. Nat. Immunol. 16, 27-35 (2015).
37. R. Hai et al., Influenza A(H7N9) virus gains neuraminidase inhibitor resistance without loss of in vivo virulence or transmissibility. Nat Commun 4, 2854 (2013).
38. C. Moore et al., Evidence of person-to-person transmission of oseltamivir-resistant pandemic influenza A(H1N1) 2009 virus in a hematology unit. J. Infect. Dis. 203, 18-24 (2011).
39. A. Hidalgo, A. Cruz, J. Perez-Gil, Barrier or carrier? Pulmonary surfactant and drug delivery. Eur. J. Pharm. Biopharm. 95, 117-127 (2015).
40. B. Olsson et al., in Controlled Pulmonary Drug Delivery, A. J. H. Hugh D. C. Smyth, Ed. (Springer-Verlag New York, N.Y., 2011), pp. 21-50.
41. J. C. Sung, B. L. Pulliam, D. A. Edwards, Nanoparticles for drug delivery to the lungs. Trends Biotechnol. 25, 563-570 (2007).
42. I. J. Bakken et al., Febrile seizures after 2009 influenza A (H1N1) vaccination and infection: a nationwide registry-based study. BMC Infect. Dis. 15, 506-015-1263-1267 (2015).
43. F. Szoka, Jr., D. Papahadjopoulos, Procedure for preparation of liposomes with large internal aqueous space and high capture by reverse-phase evaporation. Proc. Natl. Acad. Sci. U.S.A 75, 4194-4198 (1978).
44. Y. Matsuoka, E. W. Lamirande, K. Subbarao, The ferret model for influenza. Curr. Protoc. Microbiol. Chapter 15, Unit 15G 12 (2009).
45. J. Wang et al., Physical activation of innate immunity by spiky particles. Nat. Nanotechnol. 13, 1078-1086 (2018).
46. C. L. Schengrund, X. Chi, J. Sabol, J. W. Griffith, Long-term effects of instilled mineral dusts on pulmonary surfactant isolated from monkeys. Lung 173, 197-208 (1995).
47. L. Wang, M. P. Pileni, Encapsulation of Zwitterionic Au Nanocrystals into Liposomes by Reverse Phase Evaporation Method: Influence of the Surface Charge. Langmuir 32, 12370-12377 (2016).
48. A. V. Li et al., Generation of effector memory T cell-based mucosal and systemic immunity with pulmonary nanoparticle vaccination. Sci. Transl. Med. 5, 204ra130 (2013).
49. G. Matute-Bello et al., An official American Thoracic Society workshop report: features and measurements of experimental acute lung injury in animals. Am. J. Respir. Cell Mol. Biol. 44, 725-738 (2011).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising
a nanoparticle with an average size of 200-400 nm, comprising
a plurality of pulmonary surfactant-biomimetic molecules, wherein the nanoparticle is negatively charged; and
one or more cargo molecules that are enveloped by the nanoparticle, wherein the cargo molecule has a molecular weight up to 1200 Da.

2. The composition of claim 1, wherein the pulmonary surfactant-biomimetic molecules comprise 50%-90% of 1,2-dipalmitoyl-sn-glycero-3-phosphocholine (DPPC) by weight, 5%-15% of a negatively charged lipid by weight, and/or 5%-15% of a neutral lipid by weight.

3. The composition of claim 2, wherein the negatively charged lipid is 1,2-dipalmitoyl-sn-glycero-3-phospho-(1′-rac-glycerol) (DPPG) and the neutral lipid is cholesterol.

4. The composition of claim 1, wherein the nanoparticle further comprises a plurality of polyethylene glycol (PEG) with an average molecular weight of 500-5000 Da, wherein the polyethylene glycol is linked to an external surface of the nanoparticle.

5. The composition of claim 1, wherein the nanoparticle further comprises 5-15% of 1,2-dipalmitoyl-sn-glycero-3-phosphoethanolamine-N-[methoxy(polyethylene glycol)-2000] (DPPE-PEG2000) by weight.

6. The composition of claim 1, wherein the cargo molecule is a stimulator of interferon genes (STING) agonist.

7. The composition of claim 6, wherein the STING agonist is or comprises cyclic Guanosine monophosphate [GMP]-Adenosine monophosphate [AMP] (cGAMP).

8. The composition of claim 7, wherein the cGAMP is present in a concentration of 10-100 μg/ml.

9. The composition of claim 1, wherein the cargo molecule is long acting-β2-agonists (LABAs); cortisosteroids (ICS); leukotriene-pathway modulators; inhibitors targeting kinases; agonists or antagonists of receptors; agonists or antagonists of ion channels; inducers of IFN-α; long-acting muscarinic antagonists/anticholinergics (LAMAs); inhibitors against IL-5, IL-13, IL-33, or thymic stromal lymphopoietin; CXCR2 antagonists; molecules blocking proinflammatory cytokines; molecules blocking IL-17/T_{H}17; macrolides; molecules activating HDAC2; STAT6 inhibitors; anti-virus small molecule drug; Favipiravir (T705); agonists for intracellular Toll-like receptor TLR3; agonists for Nodinitib (NOD1), NOD2, NLPR3 or NPLRC3; TLR7 or TLR8 agonists; TLR8 agonists; and/or TLR9 agonists.

10. A method of promoting an immune response to an antigen, the method comprising administering to a subject an effective amount of the composition of claim 1; and administering to the subject the antigen.

11. The method of claim 10, wherein the subject is a mammal.

12. The method of claim 10, wherein the antigen is enveloped within the nanoparticle; the nanoparticle and antigen are administered in a single composition; or the nanoparticle and antigen are administered in separate compositions.

13. A method of treating a subject who has influenza, the method comprising administering to the subject a therapeutically effective amount of the composition of claim 1; and administering to the subject an antigen, wherein the cargo molecule is cGAMP and the antigen is an influenza vaccine.

14. The method of claim 13, wherein the subject is a human and the antigen is a human influenza vaccine.

15. A method of treating a subject who has airway disease, the method comprising administering to the subject a therapeutically effective amount of the composition of claim 1, wherein the cargo molecule is long acting-β2-agonists (LABAs); cortisosteroids (ICS); leukotriene-pathway modulators; inhibitors targeting kinases; agonists or antagonists of receptors; agonists or antagonists of ion channels; inducers of IFN-α; long-acting muscarinic antagonists/anticholinergics (LAMAs); inhibitors against IL-5, IL-13, IL-33, or thymic stromal lymphopoietin; CXCR2 antagonists; molecules blocking proinflammatory cytokines; molecules blocking IL-17/T_{H}17; macrolides; molecules activating HDAC2; STAT6 inhibitors; anti-virus small molecule drug; and/or Favipiravir (T705).

16. The method of claim 15, wherein the subject is a human and the airway disease is one or a combination of asthma, chronic obstructive pulmonary disease (COPD), allergy, or lung viral infection.

17. A method of treating a subject who has cancer, the method comprising administering to a subject a therapeutically effective amount of a composition of claim 1, wherein the cargo molecule is a chemotherapy agent.

18. The method of claim 17, wherein the subject is a mammal.

19. The method of claim 17, wherein the cancer is a lung cancer and the chemotherapy agent is Gefitinib, Erlotinib, Crizotinib, Everolimus, Afatinib, Crizotinib Doxorubicin, etoposide, Opdivo, and/or Trexall.

20. The method of claim 17, wherein the cancer is nasopharyngeal cancer and the chemotherapy agent is Cisplatin, Carboplatin, Gemcitabine, Doxorubicin, and/or D5-fluorouracil (5-FU).

21. The method of claim 17, wherein the cancer is trachea cancer and the chemotherapy agent is etoposide, cisplatin, and/or carboplatin.

22. The method of claim 17, wherein the cancer is bronchial cancer and the chemotherapy agent is etoposide, cisplatin, carboplatin, 5-FU, docetaxel, paclitaxel, and/or epirubicin.
